SEQUENCE LISTING (273 sequences)

SEQ ID NO: 1 - protein, 201 aa, synthetic construct
MPVQLAPGRVYNAPAFITGVTPLSDIQLSHEYWMRHALTLAKRAWDAREVPVGAVLVHNNRAIGEGWNRPIGHHDPTAHAEIMALRQGGLVLENYRLLDATLYVTLEPCVMCAGAMVHSRIARVVFGARDAKTGAAGSLMDVLHHPGMNHRVEVTEGVLGEECAALLSEFFRMRRREIKALKRASAQTDEGQSAAAGPGER

SEQ ID NO: 2-3 - skipped (2 entries)

SEQ ID NO: 4 - protein, 184 aa, synthetic construct
MYNAPRFSTGVDALSETELNHEYWMRHALNLAQRAWEEGEVPVGAVLVYQDKVIGEGWNRPIGRHDPTAHAEIMALRQGGMVLQNYRLIDATLYVTLEPCVMCAGAMIHSRIGRVVFGARDAKTGAAGSLIDVLHHPGMNHRVEVTEGVLAESCSSLLSDFFRERREQKKALKRASQDPRSSDA

SEQ ID NO: 5 - protein, 166 aa, synthetic construct
MSDTEFTHEYWMQHALTLAQRAWDEGEVPVGAVLVQDNRVIGEGWNRPIGRHDPTAHAEIMALRQGGMVLQNYRLLNTTLYVTLEPCIMCAGAMVHSRIGTLVFGARDAKTGAVGSLMNVPAHPGMNHHMQVIEGVLAPSCSALLSDFFRVRRLEKKAQKEASRKD

SEQ ID NO: 6 - protein, 166 aa, synthetic construct
MSETEFSHEYWMRHALTLAQRAWDEGEVPVGAVLVQDNRVIGEGWNRPIGRHDPTAHAEIMALRQGGMVLQNYRLLNTTLYVTLEPCIMCAGAMVHSRIGTLVFGARDAKTGAVGSLMNIPAHPQMNHHMQIIEGVLAPTCSALLSEFFRVRRLEKKAQKEALRKD

SEQ ID NO: 7 - protein, 166 aa, synthetic construct
MSDNHDHEYWMQHALTLARRAWDEGEVPVGAVLVRNGEIIGEGWNRPIGRHDPTAHAEIMALRQGGVVLQNYRLLDATLYVTLEPCVMCAGAMVHSRIGRLVFGARDAKTGAAGSLMDVLAHPGMNHRVEVTEGVLAPACSTLLSDFFRERRLQQKALKEASRKPE

SEQ ID NO: 8 - protein, 173 aa, synthetic construct
MTTPALTHEYWMNYALTLARRAWDEGEVPVGAVLVYDNRVIGEGWNRSIGKHDPTAHAEIMALRQGGMVQQNYRLLDTTLYVTLEPCVMCAGAMIHSRIGTLVFGARDAKTGAVGSQMDILNHPGMNHQVQIIEGVLAPQCSALLSDFFRMRRKEKKALKIASRDPHPAAPQR

SEQ ID NO: 9 - protein, 165 aa, synthetic construct
MDNHDHEYWMQHALTLAQRAWEEGEVPVGAVLVLNGQAIGEGWNRPIGRHDPTAHAEMMALRQGGAVLQNYRLLNATLYVTLEPCVMCAGAMVHSRIARLVFGARDAKTGAAGSLLDVLAHPGMNHRVAVTEGVLAPACSALLSDFFRQRRQQQKALKDANRKRD

SEQ ID NO: 10 - protein, 169 aa, synthetic construct
MSDNNDEFWMRHALTLARRAWQEGEVPVGAVLVHEGRVIGEGWNRPIGHHDPTAHAEIMALRQGGKVIENYRLLDTTLYVTLEPCVMCAGAMVHGRVGRLVFGARDAKTGAAGSLIDVLSHPGMNHQVQVDEGVLAEECAAMLSDFFRQRRAEKKALRQQQQQGTSSAE

SEQ ID NO: 11 - protein, 166 aa, synthetic construct
MTDYNDELWMRHALTLARRAWDEGEVPVGAVLVQDNRVIGEGWNRPIGHHDPTAHAEMMALRQGGKVLENYRLLDTTLYVTLEPCVMCAGAIVHSRIGRLVFGARDGKTGAAGSLIDILGHPGMNHQVIVSEGILAETCAGMLSDFFRQRREEKKALRKTRETPDR

SEQ ID NO: 12 - protein, 164 aa, synthetic construct
MSDNNDEYWMRHALMLARRAWDEGEVPVGAVLVHEGRVIGEGWNRPIGHHDPTAHAEIMALRQGGKVIENYRLLDTTLYVTLEPCVMCAGAMVHSRIGRLVFGARDEKTGAAGSLLDVLGHPGMNHQVQIEEGILAAECAAMLSDFFRHRRAEKKALRQAGKLL

SEQ ID NO: 13 - skipped

SEQ ID NO: 14 - protein, 171 aa, synthetic construct
MLPGVVCVTQEQDEYWMRRALTLAQRAWEQGEVPVGAVLVQGDRVIGEGWNRPIGQHDPTAHAEIMALRQGGKVLENYRLLNTTLYVTLEPCIMCAGAMVHSRIGRLVYGAHDVKTGAAGSLIDILGHPGMNHQVALHQGVLEEECAAMLSDFFRMRRQQQKALRQAQRES

SEQ ID NO: 15 - protein, 164 aa, synthetic construct
MNPQTDEYWMRYALELAKRAWEQGEVPVGAVLVQGDKVIGEGWNRPIGQHDPTAHAEIMALRQGGKVLENYRLLDTTLYVTLEPCVMCAGAMVHGRITRLVYGAKDDKTGAAGSLLDVIGHPGMNHQIQIDCGVLAEECAGMLSDFFRMRREQKKALRQAQRTG

SEQ ID NO: 16 - protein, 164 aa, synthetic construct
MNQQRDEYWMRHALGLARLAWEQGEVPVGAVLVQDERVIGEGWNRPIGQHDPTAHAEMMALRQGGKVLENYRLLNTTLYVTLEPCVMCAGAMVHSRITRLVYGAHDVKTGAAGSLLDVLGHPGMNHQIELHSGVLAEECAAMLSDFFRMRREQKKALRQAQRQS

SEQ ID NO: 17 - protein, 164 aa, synthetic construct
MNQQHDEYWMRHALGLARLAWEQGEVPVGAVLVQADRVIGEGWNRPIGQHDPTAHAEMMALRQGGKVLENYRLLNTTLYVTLEPCVMCAGAMVHSRITRLVYGAHDVKTGAAGSLLDVLGHPGMNHQVELHSGVLADECAAMLSDFFQMRREQKKALRQAQRQG

SEQ ID NO: 18-23 - skipped (6 entries)

SEQ ID NO: 24 - protein, 212 aa, synthetic construct
MEQIKLDPKTLHAKTNSSDELGCDEVSADVLAGEQPCLEQASLEQARVDEHWMRVAMTMAEKAEAAGEVPVGAVLVKDGQQIAAGYNLSISEHDPCAHAEIQCLRAAGQTIENYRLLDTTLYVTLEPCAMCAGAMVHSRIARVVFGAKDEKTGAAGTVLNLLQHPAFNHQVEVTSGVLAQECADQLSRFFKRRREEKKALKQAQKAQQGTLS

SEQ ID NO: 25 - protein, 170 aa, synthetic construct
MTESETDHIRWMRHALSLAQRAWDEGEVPVGAVLVYQGQVIGEGWNRPIGHHDPTAHAEMMALRQGGIVLQNYRLLDTTLYVTLEPCVMCAGAMVHSRIGQLIYGASDVKTGAAGSLMDVLGHPGMNHKVSVAGGVLAQECAGLLSDFFRMRRQVHKANKQAARQPSEDQ

SEQ ID NO: 26 - protein, 170 aa, synthetic construct
MTGSETDHIRWMRHALTLAQRAWDEGEVPVGAVLVYQGQVIGEGWNRPIGHHDPTAHAEMMALRQGGIVLQNYRLLDTTLYVTLEPCVMCAGAMVHSRIGQLIYGASDVKTGAAGSLMDVLGHPGMNHKVSVAGGVLAQECAGLLSDFFRMRRQVHKANKQAARQQSEDQ

SEQ ID NO: 27 - protein, 170 aa, synthetic construct
MSDTLIDEKWMRHALTLARRAWEEGEVPVGAVLVQGDTVIGEGWNRPIGHHDPTAHAEIMALRQGGKVLENYRLLDTTLYVTLEPCVMCAGAMVHGRVGRLVFGARDEKTGAAGSLLDILGHAGMNHQVRVDQGVLAAECAAMLSDFFRHRRAEKKALRDRLRAERLKGE

SEQ ID NO: 28 - protein, 163 aa, synthetic construct
MTDYNDEFWMRHALTLAQRAWDEGEVPVGAVLVQGNRVIGEGWNRPIGHHDPTAHAEIMALRQGGKVLENYRLIDTTLYVTLEPCVMCAGAIVHSRIGRLVFGARDEKTGAAGSLLDVLGHPGMNHQVKMTEGILAQQCAGMLSDFFRMRREQKKALRKSVKQ

SEQ ID NO: 29 - protein, 166 aa, synthetic construct
MTDHNDEYWMRHALRLARRAWDEGEVPVGAVLVLDNQVIGEGWNRPIGHHDPTAHAEMMALRQGGKMIENYRLLDTTLYVTLEPCVMCAGAMIHSRIGRLVYGARDAKTGAAGSLLDVLGHPGMNHQVPAECGLLNDECAAMLSDFFRQRRAEKKMLRQQQNPGRV

SEQ ID NO: 30 - protein, 164 aa, synthetic construct
MSDNNDEYWMRHALMLARRAWDEGEVPVGAVLVHEGRAIGEGWNRPIGHHDPTAHAEIMALRQGGKVIENYRLLNTTLYVTLEPCVMCAGAMVHSRIGRLVFGARDGKTGAAGSLLDVLGHPGMNHQVQIEEGILATECAAMLSDFFRHRRAEKKAQRQAGKLL

SEQ ID NO: 31 - protein, 164 aa, synthetic construct
MTQEQDEYWMRRALTLAQRAWEQGEVPVGAVLVQGDRVIGEGWNRPIGQHDPTAHAEIMALRQGGKVLENYRLLNTTLYVTLEPCIMCAGAMVHSRIGRLVYGAHDAKTGAAGSLIDILGHPGMNHQVALHQGVLEEECAAMLSDFFRMRRQQQKALRQAQRES

SEQ ID NO: 32-35 - skipped (4 entries)

SEQ ID NO: 36 - protein, 201 aa, synthetic construct
MPVQLAPGRVYNAPAFITGVTPLSDIQLSHEYWMRHALTLAKRARDAREVPVGAVLVLNNRAIGEGWNRAIGHHDPTAHAEIMALRQGGLVLENYRLLDATLYVTFEPCVMCAGAMVHSRIARVVFGVRNAKTGAAGSLMDVLHYPGMNHRVEVTEGVLGEECAALLCEFFRMPRRVFNALKRASAQTDEGQSAAAGPGER

SEQ ID NO: 37-38 - skipped (2 entries)

SEQ ID NO: 39 - protein, 184 aa, synthetic construct
MYNAPRFSTGVDALSETELNHEYWMRHALNLAQRAREEGEVPVGAVLVYQDKVIGEGWNRAIGLHDPTAHAEIMALRQGGMVLQNYRLIDATLYVTFEPCVMCAGAMIHSRIGRVVFGVRNAKTGAAGSLIDVLHYPGMNHRVEVTEGVLAESCSSLLCYFFREPREQKNALKRASQDPRSSDA

SEQ ID NO: 40 - protein, 166 aa, synthetic construct
MSDTEFTHEYWMQHALTLAQRARDEGEVPVGAVLVQDNRVIGEGWNRAIGLHDPTAHAEIMALRQGGMVLQNYRLLNTTLYVTFEPCIMCAGAMVHSRIGTLVFGVRNAKTGAVGSLMNVPAYPGMNHHMQVIEGVLAPSCSALLCYFFRVPRLVKNAQKEASRKD

SEQ ID NO: 41 - protein, 166 aa, synthetic construct
MSETEFSHEYWMRHALTLAQRARDEGEVPVGAVLVQDNRVIGEGWNRAIGLHDPTAHAEIMALRQGGMVLQNYRLLNTTLYVTFEPCIMCAGAMVHSRIGTLVFGVRNAKTGAVGSLMNIPAYPQMNHHMQIIEGVLAPTCSALLCEFFRVPRLVKNAQKEALRKD

SEQ ID NO: 42 - protein, 166 aa, synthetic construct
MSDNHDHEYWMQHALTLARRARDEGEVPVGAVLVRNGEIIGEGWNRAIGLHDPTAHAEIMALRQGGVVLQNYRLLDATLYVTFEPCVMCAGAMVHSRIGRLVFGVRNAKTGAAGSLMDVLAYPGMNHRVEVTEGVLAPACSTLLCYFFREPRLQQNALKEASRKPE

SEQ ID NO: 43 - protein, 173 aa, synthetic construct
MTTPALTHEYWMNYALTLARRARDEGEVPVGAVLVYDNRVIGEGWNRSIGKHDPTAHAEIMALRQGGMVQQNYRLLDTTLYVTFEPCVMCAGAMIHSRIGTLVFGVRNAKTGAVGSQMDILNYPGMNHQVQIIEGVLAPQCSALLCYFFRMPRKVKNALKIASRDPHPAAPQR

SEQ ID NO: 44 - protein, 165 aa, synthetic construct
MDNHDHEYWMQHALTLAQRAREEGEVPVGAVLVLNGQAIGEGWNRAIGLHDPTAHAEMMALRQGGAVLQNYRLLNATLYVTFEPCVMCAGAMVHSRIARLVFGVRNAKTGAAGSLLDVLAYPGMNHRVAVTEGVLAPACSALLCYFFRQPRQQQNALKDANRKRD

SEQ ID NO: 45 - protein, 169 aa, synthetic construct
MSDNNDEFWMRHALTLARRARQEGEVPVGAVLVLEGRVIGEGWNRAIGHHDPTAHAEIMALRQGGKVIENYRLLDTTLYVTFEPCVMCAGAMVHGRVGRLVFGVRNAKTGAAGSLIDVLSYPGMNHQVQVDEGVLAEECAAMLCYFFRQPRAVKNALRQQQQQGTSSAE

SEQ ID NO: 46 - protein, 166 aa, synthetic construct
MTDYNDELWMRHALTLARRARDEGEVPVGAVLVQDNRVIGEGWNRAIGHHDPTAHAEMMALRQGGKVLENYRLLDTTLYVTFEPCVMCAGAIVHSRIGRLVFGVRNGKTGAAGSLIDILGYPGMNHQVIVSEGILAETCAGMLCYFFRQPREVKNALRKTRETPDR

SEQ ID NO: 47 - protein, 164 aa, synthetic construct
MSDNNDEYWMRHALMLARRARDEGEVPVGAVLVLEGRVIGEGWNRAIGHHDPTAHAEIMALRQGGKVIENYRLLDTTLYVTFEPCVMCAGAMVHSRIGRLVFGVRNEKTGAAGSLLDVLGYPGMNHQVQIEEGILAAECAAMLCYFFRHPRAVKNALRQAGKLL

SEQ ID NO: 48 - skipped

SEQ ID NO: 49 - protein, 171 aa, synthetic construct
MLPGVVCVTQEQDEYWMRRALTLAQRAREQGEVPVGAVLVQGDRVIGEGWNRAIGQHDPTAHAEIMALRQGGKVLENYRLLNTTLYVTFEPCIMCAGAMVHSRIGRLVYGVHNVKTGAAGSLIDILGYPGMNHQVALHQGVLEEECAAMLCYFFRMPRQQQNALRQAQRES

SEQ ID NO: 50 - protein, 164 aa, synthetic construct
MNPQTDEYWMRYALELAKRAREQGEVPVGAVLVQGDKVIGEGWNRAIGQHDPTAHAEIMALRQGGKVLENYRLLDTTLYVTFEPCVMCAGAMVHGRITRLVYGVKNDKTGAAGSLLDVIGYPGMNHQIQIDCGVLAEECAGMLCYFFRMPREQKNALRQAQRTG

SEQ ID NO: 51 - protein, 164 aa, synthetic construct
MNQQRDEYWMRHALGLARLAREQGEVPVGAVLVQDERVIGEGWNRAIGQHDPTAHAEMMALRQGGKVLENYRLLNTTLYVTFEPCVMCAGAMVHSRITRLVYGVHNVKTGAAGSLLDVLGYPGMNHQIELHSGVLAEECAAMLCYFFRMPREQKNALRQAQRQS

SEQ ID NO: 52 - protein, 164 aa, synthetic construct
MNQQHDEYWMRHALGLARLAREQGEVPVGAVLVQADRVIGEGWNRAIGQHDPTAHAEMMALRQGGKVLENYRLLNTTLYVTFEPCVMCAGAMVHSRITRLVYGVHNVKTGAAGSLLDVLGYPGMNHQVELHSGVLADECAAMLCYFFQMPREQKNALRQAQRQG

SEQ ID NO: 53-58 - skipped (6 entries)

SEQ ID NO: 59 - protein, 212 aa, synthetic construct
MEQIKLDPKTLHAKTNSSDELGCDEVSADVLAGEQPCLEQASLEQARVDEHWMRVAMTMAEKAEAAGEVPVGAVLVKDGQQIAAGYNLSISEHDPCAHAEIQCLRAAGQTIENYRLLDTTLYVTFEPCAMCAGAMVHSRIARVVFGVKNEKTGAAGTVLNLLQYPAFNHQVEVTSGVLAQECADQLCRFFKRPREVKNALKQAQKAQQGTLS

SEQ ID NO: 60-70 - skipped (11 entries)

SEQ ID NO: 71 - protein, 201 aa, synthetic construct
MPVQLAPGRVYNAPAFITGVTPLSDIQLSHEYWMRHALTLAKRARDAREVPVGAVLVLNNRAIGEGWNRAIGHHDPTAHAEIMALRQGGLVLENYRLLDATLYVTFEPCVMCAGAMVHSRIARVVFGVRNSKRGAAGSLMNVLNYPGMNHRVEVTEGVLGEECAALLCEFYRMPRRVFNALKRASAQINEGQSAAAGPGER

SEQ ID NO: 72-73 - skipped (2 entries)

SEQ ID NO: 74 - protein, 184 aa, synthetic construct
MYNAPRFSTGVDALSETELNHEYWMRHALNLAQRAREEGEVPVGAVLVYQDKVIGEGWNRAIGLHDPTAHAEIMALRQGGMVLQNYRLIDATLYVTFEPCVMCAGAMIHSRIGRVVFGVRNSKRGAAGSLINVLNYPGMNHRVEVTEGVLAESCSSLLCDFYREPREQKNALKRASQDPRSSNA

SEQ ID NO: 75 - protein, 166 aa, synthetic construct
MSDTEFTHEYWMQHALTLAQRARDEGEVPVGAVLVQDNRVIGEGWNRAIGLHDPTAHAEIMALRQGGMVLQNYRLLNTTLYVTFEPCIMCAGAMVHSRIGTLVFGVRNSKRGAVGSLMNVPAYPGMNHHMQVIEGVLAPSCSALLCDFYRVPRLVKNAQKEASRKN

SEQ ID NO: 76 - protein, 166 aa, synthetic construct
MSETEFSHEYWMRHALTLAQRARDEGEVPVGAVLVQDNRVIGEGWNRAIGLHDPTAHAEIMALRQGGMVLQNYRLLNTTLYVTFEPCIMCAGAMVHSRIGTLVFGVRNSKRGAVGSLMNIPAYPQMNHHMQIIEGVLAPTCSALLCEFYRVPRLVKNAQKEALRKN

SEQ ID NO: 77 - protein, 166 aa, synthetic construct
MSDNHDHEYWMQHALTLARRARDEGEVPVGAVLVRNGEIIGEGWNRAIGLHDPTAHAEIMALRQGGVVLQNYRLLDATLYVTFEPCVMCAGAMVHSRIGRLVFGVRNSKRGAAGSLMNVLAYPGMNHRVEVTEGVLAPACSTLLCDFYREPRLQQNALKEASRKPE

SEQ ID NO: 78 - protein, 173 aa, synthetic construct
MTTPALTHEYWMNYALTLARRARDEGEVPVGAVLVYDNRVIGEGWNRSIGKHDPTAHAEIMALRQGGMVQQNYRLLDTTLYVTFEPCVMCAGAMIHSRIGTLVFGVRNSKRGAVGSQMNILNYPGMNHQVQIIEGVLAPQCSALLCDFYRMPRKVKNALKIASRDPHPAAPQR

SEQ ID NO: 79 - protein, 165 aa, synthetic construct
MDNHDHEYWMQHALTLAQRAREEGEVPVGAVLVLNGQAIGEGWNRAIGLHDPTAHAEMMALRQGGAVLQNYRLLNATLYVTFEPCVMCAGAMVHSRIARLVFGVRNSKRGAAGSLLNVLAYPGMNHRVAVTEGVLAPACSALLCDFYRQPRQQQNALKDANRKRN

SEQ ID NO: 80 - protein, 169 aa, synthetic construct
MSDNNDEFWMRHALTLARRARQEGEVPVGAVLVLEGRVIGEGWNRAIGHHDPTAHAEIMALRQGGKVIENYRLLDTTLYVTFEPCVMCAGAMVHGRVGRLVFGVRNSKRGAAGSLINVLSYPGMNHQVQVDEGVLAEECAAMLCDFYRQPRAVKNALRQQQQQGTSSAE

SEQ ID NO: 81 - protein, 166 aa, synthetic construct
MTDYNDELWMRHALTLARRARDEGEVPVGAVLVQDNRVIGEGWNRAIGHHDPTAHAEMMALRQGGKVLENYRLLDTTLYVTFEPCVMCAGAIVHSRIGRLVFGVRNGKRGAAGSLINILGYPGMNHQVIVSEGILAETCAGMLCDFYRQPREVKNALRKTRETPNR

SEQ ID NO: 82 - protein, 164 aa, synthetic construct
MSDNNDEYWMRHALMLARRARDEGEVPVGAVLVLEGRVIGEGWNRAIGHHDPTAHAEIMALRQGGKVIENYRLLDTTLYVTFEPCVMCAGAMVHSRIGRLVFGVRNEKRGAAGSLLNVLGYPGMNHQVQIEEGILAAECAAMLCDFYRHPRAVKNALRQAGKLL

SEQ ID NO: 83 - skipped

SEQ ID NO: 84 - protein, 171 aa, synthetic construct
MLPGVVCVTQEQDEYWMRRALTLAQRAREQGEVPVGAVLVQGDRVIGEGWNRAIGQHDPTAHAEIMALRQGGKVLENYRLLNTTLYVTFEPCIMCAGAMVHSRIGRLVYGVHNVKRGAAGSLINILGYPGMNHQVALHQGVLEEECAAMLCDFYRMPRQQQNALRQAQRES

SEQ ID NO: 85 - protein, 164 aa, synthetic construct
MNPQTDEYWMRYALELAKRAREQGEVPVGAVLVQGDKVIGEGWNRAIGQHDPTAHAEIMALRQGGKVLENYRLLDTTLYVTFEPCVMCAGAMVHGRITRLVYGVKNDKRGAAGSLLNVIGYPGMNHQIQIDCGVLAEECAGMLCDFYRMPREQKNALRQAQRIG

SEQ ID NO: 86 - protein, 164 aa, synthetic construct
MNQQRDEYWMRHALGLARLAREQGEVPVGAVLVQDERVIGEGWNRAIGQHDPTAHAEMMALRQGGKVLENYRLLNTTLYVTFEPCVMCAGAMVHSRITRLVYGVHNVKRGAAGSLLNVLGYPGMNHQIELHSGVLAEECAAMLCDFYRMPREQKNALRQAQRQS

SEQ ID NO: 87 - protein, 164 aa, synthetic construct
MNQQHDEYWMRHALGLARLAREQGEVPVGAVLVQADRVIGEGWNRAIGQHDPTAHAEMMALRQGGKVLENYRLLNTTLYVTFEPCVMCAGAMVHSRITRLVYGVHNVKRGAAGSLLNVLGYPGMNHQVELHSGVLADECAAMLCDFYQMPREQKNALRQAQRQG

SEQ ID NO: 88-93 - skipped (6 entries)

SEQ ID NO: 94 - protein, 212 aa, synthetic construct
MEQIKLDPKTLHAKTNSSDELGCDEVSADVLAGEQPCLEQASLEQARVDEHWMRVAMTMAEKAEAAGEVPVGAVLVKDGQQIAAGYNLSISEHDPCAHAEIQCLRAAGQTIENYRLLDTTLYVTFEPCAMCAGAMVHSRIARVVFGVKNEKRGAAGTVLNLLQYPAFNHQVEVTSGVLAQECADQLCRFYKRPREVKNALKQAQKAQQGILS

SEQ ID NO: 95-105 - skipped (11 entries)

SEQ ID NO: 106 - DNA, 9062 bp, synthetic construct
  source 1..9062: mol_type = other DNA
tctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaacttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgctgcttcgcgatgtacgggccagatatattaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatatgcagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtcggaagcggagctactaacttcagcctgctgaagcaggctggagacgtggaggagaaccctggacctatgagcgagctgatcaaggagaacatgcacatgaagctgtacatggagggcaccgtgaacaaccaccacttcaagtgcacatccgagggcgaaggcaagccctacgagggcacccagaccatgaagatcaaggtggtcgagggcggccctctccccttcgccttcgacatcctggctaccagcttcatgtacggcagcaaagccttcatcaaccacacccagggcatccccgacttctttaagcagtccttccctgagggcttcacatgggagagaatcaccacatacgaagacgggggcgtgctgaccgctacccaggacaccagcttccagaacggctgcatcatctacaacgtcaagatcaacggggtgaacttcccatccaacggccctgtgatgcagaagaaaacacgcggctgggaggccaacaccgagatgctgtaccccgctgacggcggcctgagaggccacagccagatggccctgaagctcgtgggcgggggctacctgcactgctccttcaagaccacatacagatccaagaaacccgctaagaacctcaagatgcccggcttccacttcgtggaccacagactggaaagaatcaaggaggccgacaaagagacctacgtcgagcagcacgagatggctgtggccaagtactgcgacctccctagcaaactggggcacagatctggtggttctcccaagaagaagaggaaagtctaa

SEQ ID NO: 107 - RNA, 20 nt, synthetic construct
  source 1..20: mol_type = other RNA
cacacacacttagaatctgt

SEQ ID NO: 108 - RNA, 20 nt, synthetic construct
  source 1..20: mol_type = other RNA
gcagagagtcgccgtctcca

SEQ ID NO: 109 - RNA, 20 nt, synthetic construct
  source 1..20: mol_type = other RNA
cccgcaccttggcgcagcgg

SEQ ID NO: 110 - RNA, 20 nt, synthetic construct
  source 1..20: mol_type = other RNA
gtttagagtgagccatgtag

SEQ ID NO: 111-112 - skipped (2 entries)

SEQ ID NO: 113 - RNA, 22 nt, synthetic construct
  source 1..22: mol_type = other RNA
gtgtcaggtaatgtgctaaaca

SEQ ID NO: 114 - skipped

SEQ ID NO: 115 - RNA, 20 nt, synthetic construct
  source 1..20: mol_type = other RNA
tctgcttctccagccctggc

SEQ ID NO: 116 - protein, 170 aa, synthetic construct
MTESETDHIRWMRHALSLAQRARDEGEVPVGAVLVYQGQVIGEGWNRAIGHHDPTAHAEMMALRQGGIVLQNYRLLDTTLYVTFEPCVMCAGAMVHSRIGQLIYGVSNVKRGAAGSLMNVLGYPGMNHKVSVAGGVLAQECAGLLCDFYRMPRQVHNANKQAARQPSENQ

SEQ ID NO: 117 - protein, 170 aa, synthetic construct
MTGSETDHIRWMRHALTLAQRARDEGEVPVGAVLVYQGQVIGEGWNRAIGHHDPTAHAEMMALRQGGIVLQNYRLLDTTLYVTFEPCVMCAGAMVHSRIGQLIYGVSNVKRGAAGSLMNVLGYPGMNHKVSVAGGVLAQECAGLLCDFYRMPRQVHNANKQAARQQSENQ

SEQ ID NO: 118 - protein, 170 aa, synthetic construct
MSDTLIDEKWMRHALTLARRAREEGEVPVGAVLVQGDTVIGEGWNRAIGHHDPTAHAEIMALRQGGKVLENYRLLDTTLYVTFEPCVMCAGAMVHGRVGRLVFGVRNEKRGAAGSLLNILGYAGMNHQVRVDQGVLAAECAAMLCDFYRHPRAVKNALRDRLRAERLKGE

SEQ ID NO: 119 - protein, 163 aa, synthetic construct
MTDYNDEFWMRHALTLAQRARDEGEVPVGAVLVQGNRVIGEGWNRAIGHHDPTAHAEIMALRQGGKVLENYRLIDTTLYVTFEPCVMCAGAIVHSRIGRLVFGVRNEKRGAAGSLLNVLGYPGMNHQVKMTEGILAQQCAGMLCDFYRMPREQKNALRKSVKQ

SEQ ID NO: 120 - protein, 166 aa, synthetic construct
MTDHNDEYWMRHALRLARRARDEGEVPVGAVLVLDNQVIGEGWNRAIGHHDPTAHAEMMALRQGGKMIENYRLLDTTLYVTFEPCVMCAGAMIHSRIGRLVYGVRNSKRGAAGSLLNVLGYPGMNHQVPAECGLLNDECAAMLCDFYRQPRAVKNMLRQQQNPGRV

SEQ ID NO: 121 - protein, 164 aa, synthetic construct
MSDNNDEYWMRHALMLARRARDEGEVPVGAVLVLEGRAIGEGWNRAIGHHDPTAHAEIMALRQGGKVIENYRLLNTTLYVTFEPCVMCAGAMVHSRIGRLVFGVRNGKRGAAGSLLNVLGYPGMNHQVQIEEGILATECAAMLCDFYRHPRAVKNAQRQAGKLL

SEQ ID NO: 122 - protein, 164 aa, synthetic construct
MTQEQDEYWMRRALTLAQRAREQGEVPVGAVLVQGDRVIGEGWNRAIGQHDPTAHAEIMALRQGGKVLENYRLLNTTLYVTFEPCIMCAGAMVHSRIGRLVYGVHNSKRGAAGSLINILGYPGMNHQVALHQGVLEEECAAMLCDFYRMPRQQQNALRQAQRES

SEQ ID NO: 123 - protein, 201 aa, synthetic construct
MPVQLAPGRVYNAPAFITGVTPLSDIQLSHEYWMRHALTLAKRARDAREVPVGAVLVLNNRAIGEGWNRAIGHHDPTAHAEIMALRQGGLVLENYRLLDATLYVTFEPCVMCAGAMVHSRIARVVFGVRQSKRGAAGSLMNVLNYPGMNHRVEVTEGVLGEECAALLCEFYRMPRRVFNALKRASAQINEGQSAAAGPGER

SEQ ID NO: 124 - protein, 184 aa, synthetic construct
MYNAPRFSTGVDALSETELNHEYWMRHALNLAQRAREEGEVPVGAVLVYQDKVIGEGWNRAIGLHDPTAHAEIMALRQGGMVLQNYRLIDATLYVTFEPCVMCAGAMIHSRIGRVVFGVRQSKRGAAGSLINVLNYPGMNHRVEVTEGVLAESCSSLLCDFYREPREQKNALKRASQDPRSSNA

SEQ ID NO: 125 - protein, 201 aa, synthetic construct
MPVQLAPGRVYNAPAFITGVTPLSDIQLSHEYWMRHALTLAKRARDARAAPVGAVLVLNNRAIGEGWNRAIGHHDPTAHAEIIALRQGGLVLENYRLLDATLYVTFEPCVMCAGAMVNSRIARVVFGVRNSKRGAAGSLMNVLNYPGMNHRVEVTEGVLGEECAALLCEFYRMPRRVFNALKRASAQINEGQSAAAGPGER

SEQ ID NO: 126 - protein, 201 aa, synthetic construct
MPVQLAPGRVYNAPAFITGVTPLSDIQLSHEYWMRHALTLAKRARDAGAGPVGAVLVLNNRAIGEGWNRAIGHHDPTAHAEIMALRQGGLVLENYRLFDATLYVTFEPCVMCAGAMVNSRIARVVFGVRNSKRGAAGSLMNVLNYPGMNHRVEVTEGVLGEECAALLCEFYRMPRRVFNSLKRASAQINEGQSAAAGPGER

SEQ ID NO: 127 - protein, 184 aa, synthetic construct
MYNAPRFSTGVDALSETELNHEYWMRHALNLAQRAREEGAAPVGAVLVYQDKVIGEGWNRAIGLHDPTAHAEIIALRQGGMVLQNYRLIDATLYVTFEPCVMCAGAMINSRIGRVVFGVRNSKRGAAGSLINVLNYPGMNHRVEVTEGVLAESCSSLLCDFYREPREQKNALKRASQDPRSSNA

SEQ ID NO: 128 - protein, 184 aa, synthetic construct
MYNAPRFSTGVDALSETELNHEYWMRHALNLAQRAREEGAGPVGAVLVYQDKVIGEGWNRAIGLHDPTAHAEIMALRQGGMVLQNYRLFDATLYVTFEPCVMCAGAMINSRIGRVVFGVRNSKRGAAGSLINVLNYPGMNHRVEVTEGVLAESCSSLLCDFYREPREQKNSLKRASQDPRSSNA

SEQ ID NO: 129 - protein, 166 aa, synthetic construct
MSDTEFTHEYWMQHALTLAQRARDEGAAPVGAVLVQDNRVIGEGWNRAIGLHDPTAHAEIIALRQGGMVLQNYRLLNTTLYVTFEPCIMCAGAMVNSRIGTLVFGVRNSKRGAVGSLMNVPAYPGMNHHMQVIEGVLAPSCSALLCDFYRVPRLVKNAQKEASRKN

SEQ ID NO: 130 - protein, 166 aa, synthetic construct
MSDTEFTHEYWMQHALTLAQRARDEGAGPVGAVLVQDNRVIGEGWNRAIGLHDPTAHAEIMALRQGGMVLQNYRLFNTTLYVTFEPCIMCAGAMVNSRIGTLVFGVRNSKRGAVGSLMNVPAYPGMNHHMQVIEGVLAPSCSALLCDFYRVPRLVKNSQKEASRKN

SEQ ID NO: 131 - protein, 173 aa, synthetic construct
MTTPALTHEYWMNYALTLARRARDEGAAPVGAVLVYDNRVIGEGWNRSIGKHDPTAHAEIIALRQGGMVQQNYRLLDTTLYVTFEPCVMCAGAMINSRIGTLVFGVRNSKRGAVGSQMNILNYPGMNHQVQIIEGVLAPQCSALLCDFYRMPRKVKNALKIASRDPHPAAPQR

SEQ ID NO: 132 - protein, 173 aa, synthetic construct
MTTPALTHEYWMNYALTLARRARDEGAGPVGAVLVYDNRVIGEGWNRSIGKHDPTAHAEIMALRQGGMVQQNYRLFDTTLYVTFEPCVMCAGAMINSRIGTLVFGVRNSKRGAVGSQMNILNYPGMNHQVQIIEGVLAPQCSALLCDFYRMPRKVKNSLKIASRDPHPAAPQR

SEQ ID NO: 133 - protein, 165 aa, synthetic construct
MDNHDHEYWMQHALTLAQRAREEGAAPVGAVLVLNGQAIGEGWNRAIGLHDPTAHAEMIALRQGGAVLQNYRLLNATLYVTFEPCVMCAGAMVNSRIARLVFGVRNSKRGAAGSLLNVLAYPGMNHRVAVTEGVLAPACSALLCDFYRQPRQQQNALKDANRKRN

SEQ ID NO: 134 - protein, 165 aa, synthetic construct
MDNHDHEYWMQHALTLAQRAREEGAGPVGAVLVLNGQAIGEGWNRAIGLHDPTAHAEMMALRQGGAVLQNYRLFNATLYVTFEPCVMCAGAMVNSRIARLVFGVRNSKRGAAGSLLNVLAYPGMNHRVAVTEGVLAPACSALLCDFYRQPRQQQNSLKDANRKRN

SEQ ID NO: 135 - protein, 169 aa, synthetic construct
MSDNNDEFWMRHALTLARRARQEGAAPVGAVLVLEGRVIGEGWNRAIGHHDPTAHAEIIALRQGGKVIENYRLLDTTLYVTFEPCVMCAGAMVNGRVGRLVFGVRNSKRGAAGSLINVLSYPGMNHQVQVDEGVLAEECAAMLCDFYRQPRAVKNALRQQQQQGTSSAE

SEQ ID NO: 136 - protein, 169 aa, synthetic construct
MSDNNDEFWMRHALTLARRARQEGAGPVGAVLVLEGRVIGEGWNRAIGHHDPTAHAEIMALRQGGKVIENYRLFDTTLYVTFEPCVMCAGAMVNGRVGRLVFGVRNSKRGAAGSLINVLSYPGMNHQVQVDEGVLAEECAAMLCDFYRQPRAVKNSLRQQQQQGTSSAE

SEQ ID NO: 137 - protein, 164 aa, synthetic construct
MSDNNDEYWMRHALMLARRARDEGAAPVGAVLVLEGRVIGEGWNRAIGHHDPTAHAEIIALRQGGKVIENYRLLDTTLYVTFEPCVMCAGAMVNSRIGRLVFGVRNEKRGAAGSLLNVLGYPGMNHQVQIEEGILAAECAAMLCDFYRHPRAVKNALRQAGKLL

SEQ ID NO: 138 - protein, 164 aa, synthetic construct
MSDNNDEYWMRHALMLARRARDEGAGPVGAVLVLEGRVIGEGWNRAIGHHDPTAHAEIMALRQGGKVIENYRLFDTTLYVTFEPCVMCAGAMVNSRIGRLVFGVRNEKRGAAGSLLNVLGYPGMNHQVQIEEGILAAECAAMLCDFYRHPRAVKNSLRQAGKLL

SEQ ID NO: 139 - protein, 212 aa, synthetic construct
MEQIKLDPKTLHAKTNSSDELGCDEVSADVLAGEQPCLEQASLEQARVDEHWMRVAMTMAEKAEAAGAAPVGAVLVKDGQQIAAGYNLSISEHDPCAHAEIQCLRAAGQTIENYRLLDTTLYVTFEPCAMCAGAMVNSRIARVVFGVKNEKRGAAGTVLNLLQYPAFNHQVEVTSGVLAQECADQLCRFYKRPREVKNALKQAQKAQQGILS

SEQ ID NO: 140 - protein, 212 aa, synthetic construct
MEQIKLDPKTLHAKTNSSDELGCDEVSADVLAGEQPCLEQASLEQARVDEHWMRVAMTMAEKAEAAGAGPVGAVLVKDGQQIAAGYNLSISEHDPCAHAEIQCLRAAGQTIENYRLFDTTLYVTFEPCAMCAGAMVNSRIARVVFGVKNEKRGAAGTVLNLLQYPAFNHQVEVTSGVLAQECADQLCRFYKRPREVKNSLKQAQKAQQGILS

SEQ ID NO: 141 - RNA, 20 nt, synthetic construct
  source 1..20: mol_type = other RNA
gagtccgagcagaagaagaa

SEQ ID NO: 142 - DNA, 9806 bp, synthetic construct
  source 1..9806: mol_type = other DNA
tctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaacttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgctgcttcgcgatgtacgggccagatatattaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatatgcagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtctcctcagagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccacacgtgacactgtttatctacatcgccaggctgtatcaccacgcagacccaaggaataggcagggcctgcgcgatctgatcagctccggcgtgaccatccagatcatgacagagcaggagtccggctactgctggcggaacttcgtgaattattctcctagcaacgaggcccactggcctaggtacccacacctgtgggtgcgcctgtacgtgctggagctgtattgcatcatcctgggcctgcccccttgtctgaatatcctgcggagaaagcagccccagctgaccttctttacaatcgccctgcagtcttgtcactatcagaggctgccaccccacatcctgtgggccacaggcctgaagtctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtcggaagcggagctactaacttcagcctgctgaagcaggctggagacgtggaggagaaccctggacctatgagcgagctgatcaaggagaacatgcacatgaagctgtacatggagggcaccgtgaacaaccaccacttcaagtgcacatccgagggcgaaggcaagccctacgagggcacccagaccatgaagatcaaggtggtcgagggcggccctctccccttcgccttcgacatcctggctaccagcttcatgtacggcagcaaagccttcatcaaccacacccagggcatccccgacttctttaagcagtccttccctgagggcttcacatgggagagaatcaccacatacgaagacgggggcgtgctgaccgctacccaggacaccagcttccagaacggctgcatcatctacaacgtcaagatcaacggggtgaacttcccatccaacggccctgtgatgcagaagaaaacacgcggctgggaggccaacaccgagatgctgtaccccgctgacggcggcctgagaggccacagccagatggccctgaagctcgtgggcgggggctacctgcactgctccttcaagaccacatacagatccaagaaacccgctaagaacctcaagatgcccggcttccacttcgtggaccacagactggaaagaatcaaggaggccgacaaagagacctacgtcgagcagcacgagatggctgtggccaagtactgcgacctccctagcaaactggggcacagatctggtggttctcccaagaagaagaggaaagtctaa

SEQ ID NO: 143-199 - skipped (57 entries)

SEQ ID NO: 200 - protein, 163 aa, synthetic construct
MKQQDEYWMRHALSLARRAWEQGEVPVGAVLVQNDRVIGEGWNRPIGQHDPTAHAEIMALRQGGKILENYRLLDTTLYVTLEPCVMCAGAMVHSRIARLVYGAHDSKSGAAGSLLDVLGHPGMNHQVELHSGVLAEACAAMLSDFFRMRREQKKALRQAQRQG

SEQ ID NO: 201 - protein, 207 aa, synthetic construct
MSDQNSNRPTPNFDLANKQTQEKEAVQEPLTEIAMEDIATEEDIMWMRHALTLADKAESIGEVPVGACVVLNGELIGEGFNTPITDNDPSAHAELRAVKEAAAAVQNYRLIDATLYVTLEPCSMCAGMLVHARVKRVVFGAKDAKTGAAGSVMNLLQHPALNHQLEVVSGVLADECANKLSDFFRKRRKEIKAAKKAKRLLEGDASN

SEQ ID NO: 202 - protein, 207 aa, synthetic construct
MKFKQAESEQVQSKQAEIDVVSLSSVEKAPVSLDSPHFDQSKVDKHWMRVAMAMAEKAEAEGEVPVGAVLVKDGQQIAAGYNLSISQHDPCAHAEILCLRAAGQTVENYRLLDATLYVTLEPCAMCAGAMVHSRIARVVFGARDEKTGAAGTVLNLLQHPAFNHQVEVTSGVLAQDCADQLSRFFKRRREEKKALKQAQKAQQERIS

SEQ ID NO: 203 - protein, 208 aa, synthetic construct
MEQTKLDPKTLHAKTHSSDELGCDEVSGGEQTCPKQAALEQAQVDEHWMRVAMAMAEKAEAEGEVPVGAVLVKDGQQIATGYNLSISQHDPCAHAEILCLRAAGQTVENYRLLDATLYVTLEPCAMCAGAMVHSRIARVVFGARDEKTGAAGTVLNLLQHPVFNHQVEVTSGVLAQDCADQLSRFFKRRREEKKALKQAQKAQQERIS

SEQ ID NO: 204 - protein, 208 aa, synthetic construct
MEQIKLDPKTLHAKTHSSDELGCDDVSGGEQTCPKQADLEQAQVDEHWMRVAMAMAEKAEAEGEVPVGAVLVKDGQQIAAGYNLSISQHDPCAHAEILCLRAAGQTVENYRLLDATLYVTLEPCAMCAGAMVHSRIARVVFGARDEKTGAAGTVLNLLQHPVFNHQVEVTSGVLAQDCADQLSRFFKRRREEKKALKQAQKAQQERIS

SEQ ID NO: 205 - protein, 165 aa, synthetic construct
MNPQTDEYWMRHALRLARLAWEQGEVPVGAVLVQGDTVIGEGWNRPIGQHDPTAHAEIMALRQGGKVLENYRLLDTTLYVTLEPCVMCAGAMVHSRITRLVYGAKDEKTGAAGSLLDVIGHPGMNHQIQIDSGVLAEECAAMLSDFFRMRREQKKALRQAQRDAG

SEQ ID NO: 206 - protein, 162 aa, synthetic construct
MTDEDWMQYAIKLAAKAEEQGEVPVGAVLVKDGVMLSEGWNQMISLNDPSAHAEMQAIRAASALVGNYRLPDCTLYVTLEPCSMCAGVMVHSRIKKVVFGASDLKTGAAGSVLNLLQHHCFNHQVEIVPGVLAQQCAAQLSGFFQRRRAEHKALKRIQPQAD

SEQ ID NO: 207 - protein, 170 aa, synthetic construct
MTENRDLHWMQLAMEMAQKAEALGEVPVGAVLVKDDKLIACGWNQPIAANDPCAHAEILCLRQAGSQLENYRLLDTTLYVTLEPCAMCAGAMVHARVGRLVYGAADPKTGAAGSVLDLVRHPLFNHKLAVSAGVMEQECSEQLSAFFRRRRQEQKTLKQARKLNPPAAEN

SEQ ID NO: 208 - protein, 163 aa, synthetic construct
MKQQDEYWMRHALSLARRAWEQGEVPVGAVLVQNDRVIGEGWNRPIGQHDPTAHAEIMALRQGGKVLENYRLLETTLYVTLEPCVMCAGAMVHSRITRLVYGAHDLKSGAAGSLLDVLGHPGMNHQVELASGVLAEECAAMLSDFFRMRREEKKALRQAQRQS

SEQ ID NO: 209 - protein, 186 aa, synthetic construct
MPACGIISVYNVAFFLLETRVTDRNDEYWMRHALQLARRAWNEGEVPVGAVLVLDGQVIGEGWNRPIGHHDPTAHAEMMALRQGGKVIENYRLMDTTLYVTLEPCVMCAGAMVHGRVGRLVYGARDAKTGAAGSLLDVLGHPGMNHRVRVDCGVLADECAAMLSEFFRQRRAEKKALRQRQSTDGI

SEQ ID NO: 210 - protein, 173 aa, synthetic construct
MNHTLDGHDTPDEYWMRHALTLAQRAQEEGEVPVGAVLVLDNQVIGEGWNRPIGHHDPTAHAEIMALRQGGNVLQNYRLINATLYVTLEPCVMCAGAMVHSRIGRLVYGANDEKTGAAGSLVDILRHPGMNHQITITSGVLAAECAHTLSDFFRIRRAQHKARRAQEKADGAA

SEQ ID NO: 211 - protein, 165 aa, synthetic construct
MNQSQDEYWMRHALRLARIAWEQGEVPVGAVLVQGDRVIGEGWNRPIGQHDPTAHAEIMALRQGGKVLENYRLLDTTLYVTLEPCVMCAGAMVHSRITRLVYGASDEKTGAAGSLIDVLGHPGMNHQVALHAGVLAEECAAMLSDFFRMRREQKKALRQAQRGID

SEQ ID NO: 212 - protein, 181 aa, synthetic construct
MPAAFFPLESTVSEMSDEYWMRYALQLAHRAREQGEVPVGAVLVQGSKVIGEGWNRPIGQHDPTAHAEMMALRQGGKVLENYRLLDTTLYVTLEPCIMCAGAIVHSRIGRLVFGARDEKTGAAGSLLDILGHPGMNHQVKVEHGLLAEECAAMLSDFFRMRREQKKALRQAQREAQQTPSS

SEQ ID NO: 213 - protein, 163 aa, synthetic construct
MNEQDEYWMRRAMALAARAEQEGEVPVGALVVYHGDCVGEGWNRSIGHHDATAHAEIMALRQAGAHLGNYRLLECTLYVTLEPCMMCAGAMVHSRIQRLVYGASDAKTGAVDSVLQLLATPGLNHRVDWRAGVLADACSAQLSDFFRRRRAEKKAARKQAAGG

SEQ ID NO: 214 - protein, 207 aa, synthetic construct
MSDQNSNRPTPNFDLANKQTQEKEAVQEPLTEIAMEDIATKEDIMWMRHALTLADKAESIGEVPVGACVVLNGELIGEGFNTPITDNDPSAHAELRAVKEAAAAVQNYRLIDATLYVTLEPCSMCAGMLVHARVKRVVFGAKDAKTGAAGSVMNLLQHPALNHQLEVVSGVLADECANKLSDFFRKRRKEIKAAKKAKRLLEGDASS

SEQ ID NO: 215 - protein, 164 aa, synthetic construct
MDTLADDEYWMRHALILAQRAWDEGEVPVGAVLVQGGKVIGEGWNRPIGQHDPTAHAEMMALRQGGRVLQNYRLLDTTLYITLEPCIMCAGAMVHSRISRLVYGAADAKTGAAGSLVDILRHPGMNHQVAITSGVLAEACSTLLSDFFRMRRQQQKAARAAGNA

SEQ ID NO: 216 - protein, 168 aa, synthetic construct
MNSVNDDEFWMQHALTLAQRAWDEGEVPVGAVLVLGNQVIGEGWNRSIGQHDPTAHAEIMALRQGGMVVQNYRLLNSTLYITLEPCVMCAGAMVHSRIGRLVYGAADAKTGAAGSLVDILRHPGMNHQIEITSGVLAEACSTQLSDFFRMRRKEQKARRAAAKRESLS

SEQ ID NO: 217 - protein, 165 aa, synthetic construct
MTDNNDEFWMRHALQLAQRAWEEGEVPVGAVLVHQGRVIGEGWNRPIGHHDPTAHAEMMAIRQGGKVIENYRLLDTTLYVTLEPCVMCAGAMVHSRIGRLVFGARDAKTGAAGSLLDVLGHPGMNHQVKFEHGILAEECAALLSDFFRQRRAEKKAQRQKNSGLV

SEQ ID NO: 218 - protein, 164 aa, synthetic construct
MTQEQDEYWMRRALTLAQRAWDEGEVPVGAVLVHGDRVIGEGWNRPIGQHDPTAHAEIMALRQGGKVLENYRLLDTTLYVTLEPCVMCAGAMVHSRIGRLVYGAHDVKTGAAGSLIDVLGHPGMNHQVRLDHGVLEDACAALLSDFFRMRRQQQKALRQAQRES

SEQ ID NO: 219 - protein, 162 aa, synthetic construct
MTDQQDQYWMRYALQLAQRAWQAGEVPVGAVLVHDDRIIGEGWNRPIGQHDPTAHAEIMALRQGGQALQNYRLLDATLYVTLEPCTMCAGAMVHSRIGRLVFGANDEKTGAAGSLLNVLGHPGMNHQVQVSSGILAQECAALLSDFFRMRREQKKARRSAPL

SEQ ID NO: 220 - protein, 207 aa, synthetic construct
MKFKQAESEQVQSKQAEIDVVSLSSVEKAPVSLDSPHFDQSKVDKHWMRVAMAMAEKAEAEGEVPVGAVLVKDGQQIAAGYNLSISQHDPCAHAEILCLRAAGQTVENYRLLDATLYVTFEPCAMCAGAMVHSRIARVVFGVRNEKRGAAGTVLNLLQYPAFNHQVEVTSGVLAQDCADQLCRFYKRPREVKNALKQAQKAQQERIS

SEQ ID NO: 221 - protein, 208 aa, synthetic construct
MEQTKLDPKTLHAKTHSSDELGCDEVSGGEQTCPKQAALEQAQVDEHWMRVAMAMAEKAEAEGEVPVGAVLVKDGQQIATGYNLSISQHDPCAHAEILCLRAAGQTVENYRLLDATLYVTFEPCAMCAGAMVHSRIARVVFGVRNEKRGAAGTVLNLLQYPVFNHQVEVTSGVLAQDCADQLCRFYKRPREVKNALKQAQKAQQERIS

SEQ ID NO: 222 - protein, 165 aa, synthetic construct
MNPQTDEYWMRHALRLARLAREQGEVPVGAVLVQGDTVIGEGWNRAIGQHDPTAHAEIMALRQGGKVLENYRLLDTTLYVTFEPCVMCAGAMVHSRITRLVYGVKNEKRGAAGSLLNVIGYPGMNHQIQIDSGVLAEECAAMLCDFYRMPREQKNALRQAQRDAG

SEQ ID NO: 223 - protein, 162 aa, synthetic construct
MTDEDWMQYAIKLAAKAEEQGEVPVGAVLVKDGVMLSEGWNQMISLNDPSAHAEMQAIRAASALVGNYRLPDCTLYVTFEPCSMCAGVMVHSRIKKVVFGVSNLKRGAAGSVLNLLQYHCFNHQVEIVPGVLAQQCAAQLCGFYQRPRAVHNALKRIQPQAN

SEQ ID NO: 224 - protein, 186 aa, synthetic construct
MPACGIISVYNVAFFLLETRVTDRNDEYWMRHALQLARRARNEGEVPVGAVLVLDGQVIGEGWNRAIGHHDPTAHAEMMALRQGGKVIENYRLMDTTLYVTFEPCVMCAGAMVHGRVGRLVYGVRNSKRGAAGSLLNVLGYPGMNHRVRVDCGVLADECAAMLCEFYRQPRAVKNALRQRQSTDGI

SEQ ID NO: 225 - protein, 173 aa, synthetic construct
MNHTLDGHDTPDEYWMRHALTLAQRAQEEGEVPVGAVLVLDNQVIGEGWNRAIGHHDPTAHAEIMALRQGGNVLQNYRLINATLYVTFEPCVMCAGAMVHSRIGRLVYGVNNEKRGAAGSLVNILRYPGMNHQITITSGVLAAECAHTLCDFYRIPRAQHNARRAQEKADGAA

SEQ ID NO: 226 - protein, 165 aa, synthetic construct
MNQSQDEYWMRHALRLARIAREQGEVPVGAVLVQGDRVIGEGWNRAIGQHDPTAHAEIMALRQGGKVLENYRLLDTTLYVTFEPCVMCAGAMVHSRITRLVYGVSNEKRGAAGSLINVLGYPGMNHQVALHAGVLAEECAAMLCDFYRMPREQKNALRQAQRGIN

SEQ ID NO: 227 - protein, 181 aa, synthetic construct
MPAAFFPLESTVSEMSDEYWMRYALQLAHRAREQGEVPVGAVLVQGSKVIGEGWNRAIGQHDPTAHAEMMALRQGGKVLENYRLLDTTLYVTFEPCIMCAGAIVHSRIGRLVFGVRNEKRGAAGSLLNILGYPGMNHQVKVEHGLLAEECAAMLCDFYRMPREQKKALRQAQREAQQTPSS

SEQ ID NO: 228 - protein, 164 aa, synthetic construct
MDTLADDEYWMRHALILAQRARDEGEVPVGAVLVQGGKVIGEGWNRAIGQHDPTAHAEMMALRQGGRVLQNYRLLDTTLYITFEPCIMCAGAMVHSRISRLVYGVANSKRGAAGSLVNILRYPGMNHQVAITSGVLAEACSTLLCDFYRMPRQQQNAARAAGNA

SEQ ID NO: 229 - protein, 168 aa, synthetic construct
MNSVNDDEFWMQHALTLAQRARDEGEVPVGAVLVLGNQVIGEGWNRSIGQHDPTAHAEIMALRQGGMVVQNYRLLNSTLYITFEPCVMCAGAMVHSRIGRLVYGVANSKRGAAGSLVNILRYPGMNHQIEITSGVLAEACSTQLCDFYRMPRKVQNARRAAAKRESLS

SEQ ID NO: 230 - protein, 165 aa, synthetic construct
MTDNNDEFWMRHALQLAQRAREEGEVPVGAVLVLQGRVIGEGWNRAIGHHDPTAHAEMMAIRQGGKVIENYRLLDTTLYVTFEPCVMCAGAMVHSRIGRLVFGVRNSKRGAAGSLLNVLGYPGMNHQVKFEHGILAEECAALLCDFYRQPRAVKNAQRQKNSGLV

SEQ ID NO: 231 - protein, 164 aa, synthetic construct
MTQEQDEYWMRRALTLAQRARDEGEVPVGAVLVLGDRVIGEGWNRAIGQHDPTAHAEIMALRQGGKVLENYRLLDTTLYVTFEPCVMCAGAMVHSRIGRLVYGVHNVKRGAAGSLINVLGYPGMNHQVRLDHGVLEDACAALLCDFYRMPRQQQNALRQAQRES

SEQ ID NO: 232 - protein, 162 aa, synthetic construct
MTDQQDQYWMRYALQLAQRARQAGEVPVGAVLVLDDRIIGEGWNRAIGQHDPTAHAEIMALRQGGQALQNYRLLDATLYVTFEPCTMCAGAMVHSRIGRLVFGVNNEKRGAAGSLLNVLGYPGMNHQVQVSSGILAQECAALLCDFYRMPREQKNARRSAPL

SEQ ID NO: 233 - protein, 163 aa, synthetic construct
MKQQDEYWMRHALSLARRAREQGEVPVGAVLVQNDRVIGEGWNRAIGQHDPTAHAEIMALRQGGKILENYRLLDTTLYVTFEPCVMCAGAMVHSRIARLVYGAHNSKSGAAGSLLDVLGHPGMNHQVELHSGVLAEACAAMLCRFFRMPRRQKNALRQAQRQG

SEQ ID NO: 234 - protein, 207 aa, synthetic construct
MSDQNSNRPTPNFDLANKQTQEKEAVQEPLTEIAMEDIATEEDIMWMRHALTLADKAESIGEVPVGACVVLNGELIGEGFNTAITDNDPSAHAELRAVKEAAAAVQNYRLIDATLYVTFEPCSMCAGMLVHARVKRVVVGAKNAKTGAAGSVMNLLQHPALNHQLEVVSGVLADECANKLCRFFRKPRRVFNAAKKAKRLLEGDASN

SEQ ID NO: 235 - protein, 212 aa, synthetic construct
MEQIKLDPKTLHAKTNSSDELGCDEVSADVLAGEQPCLEQASLEQARVDEHWMRVAMTMAEKAEAAGEVPVGAVLVKDGQQIAAGYNLSISEHDPCAHAEIQCLRAAGQTIENYRLLDTTLYVTFEPCAMCAGAMVHSRIARVVVGAKNEKTGAAGTVLNLLQHPAFNHQVEVTSGVLAQECADQLCRFFKRPRRVKNALKQAQKAQQGTLS

SEQ ID NO: 236 - protein, 208 aa, synthetic construct
MEQIKLDPKTLHAKTHSSDELGCDDVSGGEQTCPKQADLEQAQVDEHWMRVAMAMAEKAEAEGEVPVGAVLVKDGQQIAAGYNLSISQHDPCAHAEILCLRAAGQTVENYRLLDATLYVTFEPCAMCAGAMVHSRIARVVVGARNEKTGAAGTVLNLLQHPVFNHQVEVTSGVLAQDCADQLCRFFKRPRRVKNALKQAQKAQQERIS

SEQ ID NO: 237 - protein, 165 aa, synthetic construct
MNPQTDEYWMRHALRLARLAREQGEVPVGAVLVQGDTVIGEGWNRAIGQHDPTAHAEIMALRQGGKVLENYRLLDTTLYVTFEPCVMCAGAMVHSRITRLVYGAKNEKTGAAGSLLDVIGHPGMNHQIQIDSGVLAEECAAMLCRFFRMPRRQKNALRQAQRDAG

SEQ ID NO: 238 - protein, 162 aa, synthetic construct
MTDEDWMQYAIKLAAKAEEQGEVPVGAVLVKDGVMLSEGWNQMISLNDPSAHAEMQAIRAASALVGNYRLPDCTLYVTFEPCSMCAGVMVHSRIKKVVVGASNLKTGAAGSVLNLLQHHCFNHQVEIVPGVLAQQCAAQLCRFFQRPRRVHNALKRIQPQAD

SEQ ID NO: 239 - protein, 170 aa, synthetic construct
MTENRDLHWMQLAMEMAQKAEALGEVPVGAVLVKDDKLIACGWNQAIAANDPCAHAEILCLRQAGSQLENYRLLDTTLYVTFEPCAMCAGAMVHARVGRLVYGAANPKTGAAGSVLDLVRHPLFNHKLAVSAGVMEQECSEQLCRFFRRPRRVQKTLNQARKLNPPAAEN

SEQ ID NO: 240 - protein, 163 aa, synthetic construct
MKQQDEYWMRHALSLARRAREQGEVPVGAVLVQNDRVIGEGWNRAIGQHDPTAHAEIMALRQGGKVLENYRLLETTLYVTFEPCVMCAGAMVHSRITRLVYGAHNLKSGAAGSLLDVLGHPGMNHQVELASGVLAEECAAMLCRFFRMPRRVKNALRQAQRQS

SEQ ID NO: 241 - protein, 163 aa, synthetic construct
MNEQDEYWMRRAMALAARAEQEGEVPVGALVVYHGDCVGEGWNRSIGHHDATAHAEIMALRQAGAHLGNYRLLECTLYVTFEPCMMCAGAMVHSRIQRLVYGASNAKTGAVDSVLQLLAHPGLNHRVDWRAGVLADACSAQLCRFFRRPRRVKNAARKQAAGG

SEQ ID NO: 242 - protein, 207 aa, synthetic construct
MSDQNSNRPTPNFDLANKQTQEKEAVQEPLTEIAMEDIATKEDIMWMRHALTLADKAESIGEVPVGACVVLNGELIGEGFNTAITDNDPSAHAELRAVKEAAAAVQNYRLIDATLYVTFEPCSMCAGMLVHARVKRVVVGAKNAKTGAAGSVMNLLQHPALNHQLEVVSGVLADECANKLCRFFRKPRRVFNAAKKAKRLLEGDASS

SEQ ID NO: 243 - RNA, 20 nt, synthetic construct
  source 1..20: mol_type = other RNA
ccccaccgttgaagaaccag

SEQ ID NO: 244 - RNA, 20 nt, synthetic construct
  source 1..20: mol_type = other RNA
gagtccgagcagaagaagaa

SEQ ID NO: 245 - RNA, 20 nt, synthetic construct
  source 1..20: mol_type = other RNA
ctaattacagcgcggtgtgg

SEQ ID NO: 246 - RNA, 20 nt, synthetic construct
  source 1..20: mol_type = other RNA
caaaaggtgaagaaagaagt

SEQ ID NO: 247 - RNA, 20 nt, synthetic construct
  source 1..20: mol_type = other RNA
tcaaaaggtgaagaaagaag

SEQ ID NO: 248 - RNA, 20 nt, synthetic construct
  source 1..20: mol_type = other RNA
tcacagattatggcctgtat

SEQ ID NO: 249 - RNA, 20 nt, synthetic construct
  source 1..20: mol_type = other RNA
cccgcaccttggcgcagcgg

SEQ ID NO: 250 - RNA, 20 nt, synthetic construct
  source 1..20: mol_type = other RNA
ggctggcaggctccttgttc

SEQ ID NO: 251 - RNA, 20 nt, synthetic construct
  source 1..20: mol_type = other RNA
gcaaatcagcatgttcctca

SEQ ID NO: 252 - RNA, 16 nt, synthetic construct
  source 1..16: mol_type = other RNA
agtggaagaaggagat

SEQ ID NO: 253 - RNA, 16 nt, synthetic construct
  source 1..16: mol_type = other RNA
atcttcaccaggaagc

SEQ ID NO: 254 - RNA, 20 nt, synthetic construct
  source 1..20: mol_type = other RNA
tgactcacactcacccaaaa

SEQ ID NO: 255 - RNA, 20 nt, synthetic construct
  source 1..20: mol_type = other RNA
ctggaactgtaagagaatta

SEQ ID NO: 256 - protein, 20 aa, synthetic construct
GGSGGSGGSGGSGGSGGSGG

SEQ ID NO: 257 - protein, 20 aa, synthetic construct
GSSGSETPGTSESATPESSG

SEQ ID NO: 258 - RNA, 20 nt, synthetic construct
  source 1..20: mol_type = other RNA
gacaaaccagaagccgctcc

SEQ ID NO: 259 - RNA, 20 nt, synthetic construct
  source 1..20: mol_type = other RNA
gttcacacccatgacgaaca

SEQ ID NO: 260 - RNA, 22 nt, synthetic construct
  source 1..22: mol_type = other RNA
atttacagcctggcctttgggg

SEQ ID NO: 261 - RNA, 22 nt, synthetic construct
  source 1..22: mol_type = other RNA
ggtggaggagggtgcatggggt

SEQ ID NO: 262-263 - skipped (2 entries)

SEQ ID NO: 264 - RNA, 20 nt, synthetic construct
  source 1..20: mol_type = other RNA
atcttattcgatcatgcgaa

SEQ ID NO: 265 - RNA, 20 nt, synthetic construct
  source 1..20: mol_type = other RNA
gcttaggtggagcgcctatt

SEQ ID NO: 266 - RNA, 20 nt, synthetic construct
  source 1..20: mol_type = other RNA
ttcattaactgtggccggct

SEQ ID NO: 267 - RNA, 20 nt, synthetic construct
  source 1..20: mol_type = other RNA
gactaggcacaacagacaat

SEQ ID NO: 268 - protein, 184 aa, synthetic construct
MYNAPRFSTGVDALGETELNHEYWMRHALNLAQRAREEGEVPVGAVLVYQDKVIGEGWNRAIGLHDPTAHAEIMALRQGGMVLQNYRLIDATLYVTFEPCVMCAGAMIHSRIGRVVFGVRNSKRGAAGSLINVLNYPGMNHRVEVTEGVLAERCSSLLCDFYRVPRWQKNALKRASQDPRSSNA

SEQ ID NO: 269 - protein, 184 aa, synthetic construct
MYNAPRFSTGVDALSETELNHEYWMRHALNLAQRAREEGEVPVGAVLVYQDKVIGEGWNRAIGLHDPTAHAEIMALRQGGMVLQNYRLIDATLYVTFEPCVMCAGAMIHSRIGRVVFGVRNSKRGAAGSLINVLNYPGMNHRVEVTEGVLAESCSSLLRDFYRWPRWQKNALKRASQDPRSSNA

SEQ ID NO: 270 - protein, 164 aa, synthetic construct
MSDNNDEYWMRHALMLARRARDEGEVPVGAVLVLEGRVIGEGWNRAIGHHDPTAHAEIMALRQGGKVIENYRLLDTTLYVTFEPCVMCAGAMVHSRIGRLVFGMRNYKRGAAGSLLNVLGYPGMNHQVQIEEGILAAECAAMLCDFYRHPRAVKNALRQAGKLL

SEQ ID NO: 271 - protein, 1483 aa, synthetic construct
MKRTADGSEFESPKKKRKVMYNAPRFSTGVDALSETELNHEYWMRHALNLAQRAREEGEVPVGAVLVYQDKVIGEGWNRAIGLHDPTAHAEIMALRQGGMVLQNYRLIDATLYVTFEPCVMCAGAMIHSRIGRVVFGVRNSKRGAAGSLINVLNYPGMNHRVEVTEGVLAESCSSLLCDFYREPREQKNALKRASQDPRSSNASGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVAFLISPVKNSDGIFYDSRNYEAQENAILPKNADANGAYNIARKVLWAIGQFKKAEDEKLDKVKIAISNKEWLEYAQTSVKHSGGSKRTADGSEFEPKKKRKV

SEQ ID NO: 272 - protein, 752 aa, synthetic construct
MKRTADGSEFESPKKKRKVMYNAPRFSTGVDALSETELNHEYWMRHALNLAQRAREEGEVPVGAVLVYQDKVIGEGWNRAIGLHDPTAHAEIMALRQGGMVLQNYRLIDATLYVTFEPCVMCAGAMIHSRIGRVVFGVRNSKRGAAGSLINVLNYPGMNHRVEVTEGVLAESCSSLLCDFYREPREQKNALKRASQDPRSSNASGGSSGGSSGSETPGTSESATPESSGGSSGGSMMAVVYVISKSGKPLMPTTRCGHVRILLKEGKARVVERKPFTIQLTYESAEETQPLVLGIAPGRTNIGMSVVTESGESVFNAQIRTRNKDVPKLMKDRKQYRMAHRRLKRRCKRRRRAKAAGTAFEEGEKQRLLPGCFKPITCKSIRNKEARFNNRKRPVGWLTPTANHLLVTHLNVVKKVQKILPVAKVVLELNRFSFMAMNNPKVQRWQYQRGPLYGKGSVEEAVSMQQDGHCLFCKHGIDHYHHVVPRRKNGSETLENRVGLCEEHHRLVHTDKEWEANLASKKSGMNKKYHALSVLNQIIPYLADQLADMFPGNFCVTSGQDTYLFREEHGIPKDHYLDAYCIACSALTDAKKVSSPKGRPYMVRQFRRHDRQACHKANLNRRYYMGGKLVATNRHKAMDQKTDSLEEYRAAHSAADVSKLTVKHPSAQYKDMSRIMPGSILVSGEGKLFTLRRSEGRNKGQVNYFVSTEGIKYWARKCQYLRNNGGLQIYVSGGSKRTADGSEFEPKKKRKV

SEQ ID NO: 273 - protein, 684 aa, synthetic construct
MKRTADGSEFESPKKKRKVMYNAPRFSTGVDALSETELNHEYWMRHALNLAQRAREEGEVPVGAVLVYQDKVIGEGWNRAIGLHDPTAHAEIMALRQGGMVLQNYRLIDATLYVTFEPCVMCAGAMIHSRIGRVVFGVRNSKRGAAGSLINVLNYPGMNHRVEVTEGVLAESCSSLLCDFYREPREQKNALKRASQDPRSSNASGGSSGGSSGSETPGTSESATPESSGGSSGGSMGSPKKKRKVEFMVNKSYKFRLYPTKEQEQLLAKTFGCVRFVYNKMLEERIQIYEKFKDDKEALKKQTFPTPAKYKKEFPWLKEVDSLALANAQLNLQKAFQNFFSGRAGFPKFKNRKAKQSYTTNVVNGNIQLSDGYIKLPKLKWVKFKQHREIPAHHIIKACTIKKTKTGKYYVSILTEYEHQPVPKEIQTVVGLDFSMNGLFVDSEGKRANYPRFYRQALEKLAKEQRILSRRKKGSNRWHKQRLKVAKLHEKIANQRKDFLHKKSYELAKQYDCVVIENLNMKGMSQVLNFGKSVHDNGWGMFTTFLQYKLEEQGKKLIKIDKWFPSSKTCSCCDQVKESLSLSERTFRCECGFESDRAVNAAINIKHEGMKRLAIAGSKRPAATKKAGQAKKKKTGYPYDVPDYAYPYDVPDYAYPYDVPDYASGGSKRTADGSEFEPKKKRKV

(19) United States
(12) Patent Application Publication
(10) Pub. No.: US 2024/0409915 A1
(43) Pub. Date: Dec. 12, 2024

(54) DEAMINASES AND VARIANTS THEREOF FOR USE IN BASE EDITING

(71) Applicant: ACCUREDIT THERAPEUTICS (SUZHOU) CO., LTD., Suzhou (CN)

(72) Inventors: Feifei Duan, Suzhou (CN); Songyuan Li, Suzhou (CN); Huanle Liu, Suzhou (CN); Fuxin Shi, Winchester, MA (US); Ke Qin, Suzhou (CN); Jialin Tao, Suzhou (CN); Pengfei Hu, Suzhou (CN); Han Qiu, Suzhou (CN)

(21) Appl. No.: 18/814,865

(22) Filed: Aug. 26, 2024

Related U.S. Application Data
(63) Continuation of application No. 18/835,660, filed on Jan. 1, 0001.

(30) Foreign Application Priority Data
May 24, 2023 (WO) PCT/CN2023/095959
Jul. 18, 2023 (WO) PCT/CN2023/107820

(51) Int. Cl. C12N 9/78; A61K 38/00

(52) U.S. Cl. CPC C12N 9/78; C12Y 305/04004; A61K 38/00

(57) ABSTRACT

Provided herein are adenosine deaminases and variants thereof, nucleobase editors comprising deaminases, compositions comprising such editors, and methods of using such editors to generate modifications in target nucleobase sequences.

### CROSS-REFERENCE TO RELATED APPLICATIONS

This application is a continuation of and claims priority to U.S. application Ser. No. 18/835,660, filed on Aug. 2, 2024, which is National Phase application of and claims the benefit of priority under 35 U.S.C. § 371 to International Application No. PCT/CN2024/094938 filed May 23, 2024 which claims priority to PCT/CN2023/095959, filed May 24, 2023, entitled DEAMINASES AND VARIANTS THEREOF FOR USE IN BASE EDITING and PCT/CN2023/107820, filed Jul. 18, 2023, entitled DEAMINASES AND VARIANTS THEREOF FOR USE IN BASE EDITING, the contents of which are incorporated by reference in their entirety herein.

### SEQUENCE LISTING

This application contains a Sequence Listing that has been submitted electronically as an XML file named “53333-0009002_SL_ST26.XML.” The XML file, created on Aug. 22, 2024, is 203,748 bytes in size. The material in the XML file is hereby incorporated by reference in its entirety.

### TECHNICAL FIELD

This disclosure relates to deaminases for use with, e.g., nucleic acid-guided nucleases for making modifications in target nucleic acid sequences.

### BACKGROUND

Targeted editing of nucleic acid sequences, for example, the targeted cleavage or the targeted modification of genomic DNA, is an effective approach for the study of gene function and also has the potential to provide new therapies for human genetic diseases. Currently available base editors include cytidine base editors (e.g., BE4) that convert target C•G base pairs to T•A base pairs, and adenine base editors (e.g., ABE7.10) that convert A•T base pairs to G•C base pairs. There is a need in the art for improved base editors capable of inducing modifications within a target sequence with greater specificity and efficiency.

### SUMMARY

In a first aspect, the disclosure features an adenosine deaminase comprising an amino acid sequence that is at least 80%, 85%, 90%, 95%, 96%, 97%, 98%, 99%, or 100% identical to any one of SEQ ID NOs: 74, 82, 241, 268-270, 36, 39-47, 49-52, 59, 71, 75-81, 84-87, 94, 116-140, and 220-240, 242.. In some embodiments, the adenosine deaminase is capable of deaminating adenosine in DNA. In some embodiments, the adenosine deaminase is capable of deaminating cytosine in DNA.

In some embodiments, the adenosine deaminase comprises one or more alterations at positions S15X, L19X, N30X, A61X, G63X, M81N, V119X, S122X, S153X, S155X, C159X, E164X, E167X as numbered relative to SEQ ID NO: 74, or one or more corresponding alterations thereof, wherein X is any amino acid. In some embodiments, the adenosine deaminase comprises one or more alterations at positions S15G, L19A, N30R, A61C, G63Q, M81N, V119M, S122R, S153R, S155R, C159R, E164V, E164L, E164W, E167W as numbered relative to SEQ ID NO: 74.

In some embodiments, the adenosine deaminase comprises one or more alterations at positions V104X, E107X as numbered relative to SEQ ID NO: 82, or one or more corresponding alterations thereof, wherein X is any amino acid. In some embodiments, the adenosine deaminase comprises one or more alterations at positions V104M, E107Y, E107K as numbered relative to SEQ ID NO: 82.

In some embodiments, the adenosine deaminase comprises one or more alterations at positions G64X, L67X, N69X, Y70X, L73X, F81X, H93X, A119X, S139X, C143X as numbered relative to SEQ ID NO: 241, or one or more corresponding alterations thereof, wherein X is any amino acid. In some embodiments, the adenosine deaminase comprises one or more alterations at positions G64Y, L67Y, N69A, Y70A, Y70G, L73F, F81L, H93V, A119H, S139K, C143A as numbered relative to SEQ ID NO: 241.

In another aspect, the disclosure features a nucleobase editor comprising (i) a polynucleotide programmable DNA binding domain; and (ii) an adenosine deaminase, wherein the adenosine deaminase has an amino acid sequence that is at least 80%, 85%, 90%, 95%, 96%, 97%, 98%, or 99% identical to any one of SEQ ID NOs: 74, 82, 241, 268-270, 36, 39-47, 49-52, 59, 71, 75-81, 84-87, 94, 116-140, and 220-240, 242.. In some embodiments, the nucleobase editor further includes one or more uracil glycosylase inhibitor (UGI) domains. In some embodiments, the nucleobase editor is an adenosine base editor (ABE). In some embodiments, the nucleobase editor is a cytidine base editor (CBE). In some embodiments, the polynucleotide programmable DNA binding domain comprises a domain selected from the group consisting of a Cas9 domain, Cas 12 domain, TnpB domain, IscB domain, meganuclease domain, zinc finger DNA binding domain, and transcription activator-like effector (TALE) DNA binding domain. In some embodiments, the polynucleotide programmable DNA binding domain comprises a Cas9 domain. In some embodiments, Cas9 domain is a Staphylococcus aureus Cas9 (SaCas9), Streptococcus thermophilus 1 Cas9 (St1Cas9), a Streptococcus pyogenes Cas9 (SpCas9), or variants thereof. In some embodiments, the polynucleotide programmable DNA binding domain is a nuclease inactive or nickase variant.

In some embodiments, the base editor comprises a nucleic acid-guided nuclease protein and an inlaid nucleotide base editor (NBE) domain. In some embodiments, the inlaid NBE domain is an adenosine base editor (ABE) domain. In some embodiments, the inlaid ABE domain is an inlaid adenosine deaminase protein domain. In some embodiments, the polynucleotide programmable DNA binding domain comprises a linker that flanks at least one inlaid domain protein.

In another aspect, the disclosure features a polynucleotide encoding an adenosine deaminase that is at least 80%, 85%, 90%, 95%, 96%, 97%, 98%, or 99% identical to any one of SEQ ID NOs: 74, 82, 241, 268-270, 36, 39-47, 49-52, 59, 71, 75-81, 84-87, 94, 116-140, and 220-240, 242.

In another aspect, the disclosure features an expression vector comprising a polynucleotide encoding an adenosine deaminase that is at least 80%, 85%, 90%, 95%, 96%, 97%, 98%, or 99% identical to any one of SEQ ID NOs: 74, 82, 241, 268-270, 36, 39-47, 49-52, 59, 71, 75-81, 84-87, 94, 116-140, and 220-240, 242.. In some embodiments, the expression vector is a viral vector selected from the group consisting of adeno-associated virus (AAV), retroviral vector, adenoviral vector, lentiviral vector, Sendai virus vector, and herpesvirus vector.

In another aspect, the disclosure features a polynucleotide encoding a nucleobase editor comprising (i) a polynucleotide programmable DNA binding domain; and (ii) an adenosine deaminase, wherein the adenosine deaminase has an amino acid sequence that is at least 80%, 85%, 90%, 95%, 96%, 97%, 98%, or 99% identical to any one of SEQ ID NOs: 74, 82, 241, 268-270, 36, 39-47, 49-52, 59, 71, 75-81, 84-87, 94, 116-140, and 220-240, 242.

In another aspect, the disclosure features an expression vector comprising a polynucleotide encoding a nucleobase editor comprising (i) a polynucleotide programmable DNA binding domain; and (ii) an adenosine deaminase, wherein the adenosine deaminase has an amino acid sequence that is at least 80%, 85%, 90%, 95%, 96%, 97%, 98%, or 99% identical to any one of SEQ ID NOs: 74, 82, 241, 268-270, 36, 39-47, 49-52, 59, 71, 75-81, 84-87, 94, 116-140, and 220-240, 242.. In some embodiments, the vector is a viral vector selected from the group consisting of adeno-associated virus (AAV), retroviral vector, adenoviral vector, lentiviral vector, Sendai virus vector, and herpesvirus vector.

In another aspect, the disclosure features a method for base editing comprising contacting a polynucleotide sequence with a nucleobase editor comprising (i) a polynucleotide programmable DNA binding domain; and (ii) an adenosine deaminase, wherein the adenosine deaminase has an amino acid sequence that is at least 80%, 85%, 90%, 95%, 96%, 97%, 98%, or 99% identical to any one of SEQ ID NOs: 74, 82, 241, 268-270, 36, 39-47, 49-52, 59, 71, 75-81, 84-87, 94, 116-140, and 220-240, 242., wherein the adenosine deaminase deaminates a nucleobase in the polynucleotide, thereby editing the polynucleotide sequence.

In another aspect, the disclosure provides a method of correcting a genetic defect in a subject, the method comprising administering to the subject a nucleobase editor comprising (i) a polynucleotide programmable DNA binding domain; and (ii) an adenosine deaminase, wherein the adenosine deaminase has an amino acid sequence that is at least 80%, 85%, 90%, 95%, 96%, 97%, 98%, or 99% identical to any one of SEQ ID NOs: 74, 82, 241, 268-270, 36, 39-47, 49-52, 59, 71, 75-81, 84-87, 94, 116-140, and 220-240, 242., to deaminate a target nucleobase in a target nucleotide sequence of the subject, thereby correcting the genetic defect.

In another aspect, the disclosure provides a method of correcting a genetic defect in a subject, the method comprising administering to the subject a polynucleotide encoding a nucleobase editor comprising (i) a polynucleotide programmable DNA binding domain; and (ii) an adenosine deaminase, wherein the adenosine deaminase has an amino acid sequence that is at least 80%, 85%, 90%, 95%, 96%, 97%, 98%, or 99% identical to any one of SEQ ID NOs: 74, 82, 241, 268-270, 36, 39-47, 49-52, 59, 71, 75-81, 84-87, 94, 116-140, and 220-240, 242., to deaminate a target nucleobase in a target nucleotide sequence of the subject, thereby correcting the genetic defect.

In some embodiments the method includes delivering the nucleobase editor or the polynucleotide encoding the nucleobase editor, and one or more guide polynucleotides to a cell of the subject. In some embodiments, the subject is a mammal or a human. In some embodiments, the deamination of the target nucleobase replaces the target nucleobase with a wild type nucleobase.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a plot of A-to-G editing efficiency and indel efficiency of adenine base editors with 1^{st} round engineered TadA variants at sgRNA0 target site measured by next-generation sequencing.

FIG. 1B is a plot of A-to-G editing efficiency of adenine base editors with 1^{st} round engineered TadA variants at 3 different sgRNA target sites measured by next-generation sequencing.

FIG. 2A is a plot of A-to-G editing efficiency and indel efficiency of adenine base editors with 2^{nd} round engineered TadA variants at sgRNA0 target site measured by next-generation sequencing.

FIG. 2B is a plot of A-to-G editing efficiency of adenine base editors with 2^{nd} round engineered TadA variants at 3 different sgRNA target sites measured by next-generation sequencing.

FIG. 3A is a plot of average value of A-to-G editing efficiency relative to ABE8e across 3 or 2 sites for new adenine base editors measured by next-generation sequencing.

FIG. 3B is a plot of R-loop/on-target activity relative to ABE8e at two R-loop sites for new adenine base editors measured by next-generation sequencing.

FIG. 3C is a plot of calculated precision ratio relative to ABE8e at 2 or 1 target sites for new adenine base editors measured by next-generation sequencing.

FIG. 3D is a plot of C-to-G or T editing divided by total editing (impurity ratio) relative to ABE8e at 2 target sites for new adenine base editors measured by next-generation sequencing.

FIG. 3E is a plot of A-to-G editing efficiency and C-to-G or T editing efficiency at 2 target sites for new adenine base editor T8.24 measured by next-generation sequencing.

FIG. 4 is a plot of calculated precision ratio relative to ABE8e of T8.1, T8.4, T8.1-Q and T8.4-Q measured by next-generation sequencing.

FIG. 5 is a plot of C-to-T editing efficiency of engineered TadA derived CBEs and BE4max measured by next-generation sequencing.

FIG. 6 is a plot of mutation counts identified in survival colonies by Sanger sequencing.

FIG. 7 is a plot of percentage of mutations identified in survival colonies by next-generation sequencing.

FIG. 8 is a plot showing the fold change in percentage of mutations before and after selection.

FIG. 9 is a plot of A to G editing efficiency of T8.4 variants in library cells.

FIG. 10 is a plot of percentage of mutations identified in survival colonies by next-generation sequencing.

FIG. 11 is a plot showing the fold change in percentage of mutations before and after selection.

FIG. 12 is a is a plot of percentage of mutations identified in survival colonies after directed evolution of an NNK mutation library of the T8.4 variant as screened by next-generation sequencing.

FIG. 13 is a is a plot showing the fold change in percentage of mutations before and after selection for an NNK mutation library of the T8.4 variant as screened by next-generation sequencing.

### DETAILED DESCRIPTION

### Adenosine Base Editors

Disclosed herein are nucleobase editors, e.g., adenosine base editors, for editing, modifying or altering a target nucleotide sequence of a polynucleotide. Described herein are nucleobase editors comprising a programmable nucleotide binding domain, for example, a polynucleotide programmable nucleotide binding domain (e.g., Cas9) or zinc finger protein DNA binding domain or TALE DNA binding domain and at least one nucleobase editing domain, e.g., an adenosine deaminase. A polynucleotide programmable nucleotide binding domain (e.g., Cas9 or Cas12), when present in a cell and in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited.

In some embodiments, base editing activity is assessed by efficiency of editing. Base editing can be determined by any suitable means, for example, by Sanger sequencing or next generation sequencing. In some embodiments, base editing efficiency is measured by percentage of total sequencing reads with nucleobase conversion effected by the base editor, for example, percentage of total sequencing reads with target A-T base pair converted to a G-C base pair. In some embodiments, base editing efficiency is measured by percentage of total cells with nucleobase conversion effected by the base editor, when base editing is performed in a population of cells.

The term “base editor system” refers to the components required for editing a nucleobase of a target nucleotide sequence. In various embodiments, a base editor system comprises (1) a polynucleotide programmable nucleotide binding domain (e.g. Cas9); (2) a deaminase domain (e.g. an adenosine deaminase and/or cytidine deaminase; see PCT/US2019/044935, PCT/US2020/016288, each of which is incorporated herein by reference for its entirety) for deaminating said nucleobase; and (3) one or more guide polynucleotide (e.g., guide RNA). In some embodiments, a polynucleotide programmable nucleotide binding domain is a polynucleotide programmable DNA binding domain. In some embodiments, a base editor is an adenine or adenosine base editor (ABE).

In some embodiments, a base editor system can comprise more than one base editing component. For example, a base editor system can include more than one deaminase. In some embodiments, a base editor system can include one or more adenosine deaminases. In some embodiments, a single guide polynucleotide can be utilized to target different deaminases to a target nucleic acid sequence. In some embodiments, a pair of guide polynucleotides can be utilized to target different deaminases to a target nucleic acid sequence.

The deaminase domain and the polynucleotide programmable nucleotide binding component of a base editor system can be associated with each other covalently or noncovalently, or any combination of associations and interactions thereof. For example, in some embodiments, a deaminase domain can be targeted to a target nucleotide sequence by a polynucleotide programmable nucleotide binding domain. In some embodiments, a polynucleotide programmable nucleotide binding domain can be fused or linked to a deaminase domain. In some embodiments, a polynucleotide programmable nucleotide binding domain can target a deaminase domain to a target nucleotide sequence by noncovalently interacting with or associating with the deaminase domain. For example, in some embodiments, the deaminase domain can comprise an additional heterologous portion or domain that is capable of interacting with, associating with, or capable of forming a complex with an additional heterologous portion or domain that is part of a polynucleotide programmable nucleotide binding domain.

In some embodiments, the additional heterologous portion can be capable of binding to, interacting with, associating with, or forming a complex with a polypeptide. In some embodiments, the additional heterologous portion can be capable of binding to, interacting with, associating with, or forming a complex with a polynucleotide. In some embodiments, the additional heterologous portion can be capable of binding to a guide polynucleotide. In some embodiments, the additional heterologous portion can be capable of binding to a polypeptide linker. In some embodiments, the additional heterologous portion can be capable of binding to a polynucleotide linker. The additional heterologous portion can be a protein domain. In some embodiments, the additional heterologous portion can be a K Homology (KH) domain, a MS2 coat protein domain, a PP7 coat protein domain, a SfMu Com coat protein domain, a steril alpha motif, a telomerase Ku binding motif and Ku protein, a telomerase Sm7 binding motif and Sm7 protein, or a RNA recognition motif.

A base editor system can further include a guide polynucleotide component. It should be appreciated that components of the base editor system can be associated with each other via covalent bonds, noncovalent interactions, or any combination of associations and interactions thereof. In some embodiments, a deaminase domain can be targeted to a target nucleotide sequence by a guide polynucleotide. For example, in some embodiments, the deaminase domain can comprise an additional heterologous portion or domain (e.g., polynucleotide binding domain such as an RNA or DNA binding protein) that is capable of interacting with, associating with, or capable of forming a complex with a portion or segment (e.g., a polynucleotide motif) of a guide polynucleotide. In some embodiments, the additional heterologous portion or domain (e.g., polynucleotide binding domain such as an RNA or DNA binding protein) can be fused or linked to the deaminase domain. In some embodiments, the additional heterologous portion can be capable of binding to, interacting with, associating with, or forming a complex with a polypeptide. In some embodiments, the additional heterologous portion can be capable of binding to, interacting with, associating with, or forming a complex with a polynucleotide. In some embodiments, the additional heterologous portion can be capable of binding to a guide polynucleotide. In some embodiments, the additional heterologous portion can be capable of binding to a polypeptide linker. In some embodiments, the additional heterologous portion can be capable of binding to a polynucleotide linker. The additional heterologous portion can be a protein domain. In some embodiments, the additional heterologous portion can be a K Homology (KH) domain, a MS2 coat protein domain, a PP7 coat protein domain, a SfMu Com coat protein domain, a sterile alpha motif, a telomerase Ku binding motif and Ku protein, a telomerase Sm7 binding motif and Sm7 protein, or a RNA recognition motif.

### Guide Polynucleotides

In some embodiments, the guide polynucleotide is a guide RNA. An RNA/Cas complex can assist in “guiding” a Cas protein to a target DNA. Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA,” or simply “gRNA”) can be engineered so as to incorporate aspects of both the crRNA and tracrRNA into a single RNA species. See, e.g., Jinek M. et al., Science 337:816-821(2012), the entire contents of which is hereby incorporated by reference. Cas9 recognizes a short motif in the CRISPR repeat sequences (the PAM or protospacer adjacent motif) to help distinguish self versus non-self.

Cas9 nuclease sequences and structures are well known to those of skill in the art (see e.g., “Complete genome sequence of an Ml strain of Streptococcus pyogenes.” Ferretti, J. J. et al., Natl. Acad. Sci. U.S.A. 98:4658-4663(2001); “CRISPR RNA maturation by trans-encoded small RNA and host factor RNase III.” Deltcheva E. et al., Nature 471 :602-607(2011); and “Programmable dual-RNA-guided DNA endonuclease in adaptive bacterial immunity.” Jinek M. et al, Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences would be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain, that is, the Cas9 is a nickase. In some embodiments, the guide polynucleotide is at least one single guide RNA (“sgRNA” or “gNRA”). In some embodiments, the guide polynucleotide is at least one tracrRNA. In some embodiments, the guide polynucleotide does not require protospacer adjacent motif (PAM) sequence to guide the polynucleotide-programmable DNA-binding domain (e.g., Cas9 or Cpf1) to the target nucleotide sequence. The polynucleotide programmable nucleotide binding domain (e.g., a CRISPR-derived domain) of the base editors disclosed herein can recognize a target polynucleotide sequence by associating with a guide polynucleotide. A guide polynucleotide (e.g., gRNA) is typically single-stranded and can be programmed to site-specifically bind (i.e., via complementary base pairing) to a target sequence of a polynucleotide, thereby directing a base editor that is in conjunction with the guide nucleic acid to the target sequence. A guide polynucleotide can be DNA. A guide polynucleotide can be RNA. In some embodiments, the guide polynucleotide comprises natural nucleotides (e.g., adenosine). In some embodiments, the guide polynucleotide comprises non-natural (or unnatural) nucleotides (e.g., peptide nucleic acid or nucleotide analogs). In some embodiments, the targeting region of a guide nucleic acid sequence can be at least 15, 16, 17, 18, 19, 20, 21, 22, 23, 24, 25, 26, 27, 28, 29, or 30 nucleotides in length. A targeting region of a guide nucleic acid can be between 10-30 nucleotides in length, or between 15-25 nucleotides in length, or between 15-20 nucleotides in length.

### Programmable Nucleotide Binding Domain

A programmable nucleotide binding domain of a base editor can itself comprise one or more domains. For example, a polynucleotide programmable nucleotide binding domain can comprise one or more nuclease domains. In some embodiments, the nuclease domain of a polynucleotide programmable nucleotide binding domain can comprise an endonuclease or an exonuclease. Herein the term “exonuclease” refers to a protein or polypeptide capable of digesting a nucleic acid (e.g., RNA or DNA) from free ends, and the term “endonuclease” refers to a protein or polypeptide capable of catalyzing (e.g., cleaving) internal regions in a nucleic acid (e.g., DNA or RNA). In some embodiments, an endonuclease can cleave a single strand of a double-stranded nucleic acid.

Any DNA destabilizing molecule can be used in the compositions described herein in any combination, including but not limited to a Cas9 or Cas12 nickase, a Cas9 or Cas12 protein (e.g., dCas) operably linked to a single guide RNA (sgRNA), any RNA programmable system, a zinc finger nuclease nickase (ZFN nickase), a TALEN nickase, and/or one or more nucleotides (e.g., one or more peptide nucleic acids (PNAs), locked nucleic acids (LNAs) and/or bridged nucleic acids (BNAs)). In certain embodiments, the base editing composition comprises more than one DNA destabilizing molecule, for example one or more proteins (e.g., nickases, etc.) and/or one or more nucleotides. In certain embodiments, the composition comprises a ZFN nickase and one or more additional proteins and/or nucleotide DNA destabilizing molecules (e.g., one or more nucleotides as described herein). In certain aspects, the base editing composition does not comprise a Cas9 protein, but may comprise other Cas protein (e.g, non-Cas9 RNA programmable systems). In certain embodiments, the DNA-destabilizing molecule comprises a zinc finger nuclease (ZFN) nickase.

In some embodiments, the nuclease is a zinc finger nuclease (ZFN) or TALE DNA binding domain-nuclease fusion (TALEN). ZFNs and TALENs comprise a DNA binding domain (zinc finger protein or TALE DNA binding domain) that has been engineered to bind to a target site in a gene of choice and cleavage domain or a cleavage half domain.

The at least one zinc finger protein (ZFP) DNA-binding domain of the base editing composition can be operably linked to one or more of the other components of the base editing composition, for example to one or more of the DNA destabilizing molecules (e.g., to Cas9 nickase, dCas9, etc.) and/or to the at least one adenine or cytosine deaminase. In certain embodiments, at least one ZFP DNA-binding domain is operably linked to the adenine or cytosine deaminase. In other embodiments, the base editing composition comprises first and second ZFP DNA-binding domains, wherein the first ZFP DNA-binding domain is operably linked to the Cas9 nickase. The ZFP DNA-binding domain can comprise 3, 4, 5, 6 or more fingers and may bind to a target site on either side (5′ or 3′) of the targeted base to be edited. In certain embodiments, the ZFP binds to a target site that is 1 to 100 (or any number therebetween) nucleotides on either side of the targeted base. In other embodiments, the ZFP binds to a target site that is 1 to 50 (or any number therebetween) nucleotides on either side of the targeted base.

In some embodiments, an endonuclease can cleave both strands of a double-stranded nucleic acid molecule. In some embodiments a polynucleotide programmable nucleotide binding domain can be a deoxyribonuclease. In some embodiments a polynucleotide programmable nucleotide binding domain can be a ribonuclease.

In some embodiments, a nuclease domain of a polynucleotide programmable nucleotide binding domain can cut zero, one, or two strands of a target polynucleotide. In some embodiments, the polynucleotide programmable nucleotide binding domain can comprise a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). In some embodiments, a nickase can be derived from a fully catalytically active (e.g., natural) form of a polynucleotide programmable nucleotide binding domain by introducing one or more mutations into the active polynucleotide programmable nucleotide binding domain. For example, where a polynucleotide programmable nucleotide binding domain comprises a nickase domain derived from Cas9, the Cas9-derived nickase domain can include a D10A mutation and a histidine at position 840. In such embodiments, the residue H840 retains catalytic activity and can thereby cleave a single strand of the nucleic acid duplex. In another example, a Cas9-derived nickase domain can comprise an H840A mutation, while the amino acid residue at position 10 remains a D. In some embodiments, a nickase can be derived from a fully catalytically active (e.g., natural) form of a polynucleotide programmable nucleotide binding domain by removing all or a portion of a nuclease domain that is not required for the nickase activity. For example, where a polynucleotide programmable nucleotide binding domain comprises a nickase domain derived from Cas9, the Cas9-derived nickase domain can comprise a deletion of all or a portion of the RuvC domain or the HNH domain.

A base editor comprising a polynucleotide programmable nucleotide binding domain comprising a nickase domain is thus able to generate a single-strand DNA break (nick) at a specific polynucleotide target sequence (e.g., determined by the complementary sequence of a bound guide nucleic acid). In some embodiments, the strand of a nucleic acid duplex target polynucleotide sequence that is cleaved by a base editor comprising a nickase domain (e.g., Cas9-derived nickase domain) is the strand that is not edited by the base editor (i.e., the strand that is cleaved by the base editor is opposite to a strand comprising a base to be edited). In other embodiments, a base editor comprising a nickase domain (e.g., Cas9-derived nickase domain) can cleave the strand of a DNA molecule which is being targeted for editing. In such embodiments, the non-targeted strand is not cleaved.

Also provided herein are base editors comprising a polynucleotide programmable nucleotide binding domain which is catalytically dead (i.e., incapable of cleaving a target polynucleotide sequence). Herein the terms “catalytically dead” and “nuclease dead” are used interchangeably to refer to a polynucleotide programmable nucleotide binding domain which has one or more mutations and/or deletions resulting in its inability to cleave a strand of a nucleic acid while retaining its ability, and specificity, to bind to a target polynucleotide. In some embodiments, a catalytically dead polynucleotide programmable nucleotide binding domain base editor can lack nuclease activity as a result of specific point mutations in one or more nuclease domains. For example, in the case of a base editor comprising a Cas9 domain, the Cas9 can comprise both a D10A mutation and an H840A mutation. Such mutations inactivate both nuclease domains, thereby resulting in the loss of nuclease activity. In other embodiments, a catalytically dead polynucleotide programmable nucleotide binding domain can comprise one or more deletions of all or a portion of a catalytic domain (e.g., RuvCl and/or HNH domains). In further embodiments, a catalytically dead polynucleotide programmable nucleotide binding domain comprises a point mutation (e.g., D10A or H840A) as well as a deletion of all or a portion of a nuclease domain.

Some aspects of the disclosure provide fusion proteins comprising domains that act as polynucleotide-programmable DNA binding proteins, which can be used to guide a protein, such as a base editor, to a specific nucleic acid (e.g., DNA or RNA) sequence. In particular embodiments, a fusion protein comprises a nucleic acid programmable DNA binding protein domain and one or more deaminase domains. Non-limiting examples of polynucleotide-programmable DNA binding proteins include, Cas9 (e.g., dCas9 and nCas9), Cas12a/Cpf1, Cas12b/C2c1, Cas12c/C2c3, Cas12d/CasY, Cas12e/CasX, Cas12g, Cas12h, and Cas12i. Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx1 1, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1 :325-336. doi: 10.1089/crispr.2018.0033; Yan et al., “Functionally diverse type V CRISPR-Cas systems” Science. 2019 Jan. 4; 363(6422):88-91. doi: 10.1 126/science.aav7271, the entire contents of each are hereby incorporated by reference.

In some embodiments, the disclosure provides a fusion protein comprising a type V CRISPR/Cas effector protein. Type V CRISPR/Cas effector proteins are a subtype of Class 2 CRISPR/Cas effector proteins. For examples of type V CRISPR/Cas systems and their effector proteins (e.g., Cas12 family proteins such as Cas12a), see, e.g., Shmakov et al., Nat Rev Microbial. 2017 March; 15(3):169-182: “Diversity and evolution of class 2 CRISPR-Cas systems.” Examples include, but are not limited to: Cas12 family (Cas12a, Cas12b, Cas12c), C2c4, C2c8, C2c5, C2c10, and C2c9; as well as CasX (Cas12e) and CasY (Cas12d). Also see, e.g., Koonin et al., Curr Opin Microbial. 2017 June; 37:67-78: “Diversity, classification and evolution of CRISPR-Cas systems.” In some embodiments, the CBEs disclosed herein comprise a type V CRISPR/Cas effector protein.

In some embodiments, the disclosure provides TALE base editors, which can comprise a TALE domain, a deaminase domain and/or cofactor protein (e.g. FokI endonuclease) domain that comprise fusion proteins having the general structure NH2-[TALE]-[deaminase domain]-COOH, NH2-[deaminase domain]-[TALE]-COOH, NH2-[TALE]-[deaminase domain]-[cofactor protein]-COOH, NH2-[cofactor protein]-[deaminase domain]-[TALE]-COOH, NH2-[cofactor protein]-[TALE]-[deaminase]-COOH or NH2-[deaminase domain]-[TALE]-[cofactor protein]-COOH; wherein each instance of “]-[” comprises an optional linker, e.g. a peptide linker.

In some embodiments, the disclosed methods involve transducing (e.g. via transfection) cells with a plurality of complexes each comprising a fusion protein comprising a TAL effector domain and a deaminase domain and a cofactor protein, wherein each cofactor protein localizes the fusion protein to a distinct target sequence. Reference is made to Yang L. et al., Engineering and optimizing deaminase fusions for genome editing, Nature Comms., 2016. In particular embodiments, the methods disclosed herein involve TAL effector domains that bind target sites not by Watson-Crick hybridization, but by binding the major groove of the DNA double helix. In certain embodiments, the methods involve the transfection of nucleic acid constructs (e.g. plasmids) that each (or together) encode the components of a plurality of complexes of a TALE base editor comprising a TALE domain and a deaminase domain, and a cofactor protein. In certain embodiments, the disclosed fusion proteins comprise a cofactor protein domain—i.e. the domain is incorporated into the fusion protein construct. In other embodiments, the TALE base editor comprises a TALE domain and a deaminase domain, and the cofactor protein is introduced into the cell separately from the base editor.

In certain embodiments of the disclosed methods, the constructs that encode the TALE base editors are transfected into the cell separately from the constructs that encode the cofactor proteins. In certain embodiments, these components are encoded on a single construct and transfected together. In particular embodiments, these single constructs encoding the TALE base editor and cofactor proteins can be transfected into the cell iteratively, with each iteration associated with a subset of target sequences. In particular embodiments, these single constructs can be transfected into the cell over a period of days. In other embodiments, they can be transfected into the cell over a period of weeks.

### A to G Editing

In some embodiments, a base editor described herein can comprise a deaminase domain which includes an adenosine deaminase. Such an adenosine deaminase domain of a base editor can facilitate the editing of an adenine (A) nucleobase to a guanine (G) nucleobase by deaminating the A to form inosine (I), which exhibits base pairing properties of G. Adenosine deaminase is capable of deaminating (i.e., removing an amine group) adenine of a deoxyadenosine residue in deoxyribonucleic acid (DNA).

In some embodiments, the nucleobase editors provided herein can be made by fusing together one or more protein domains, thereby generating a fusion protein. In certain embodiments, the fusion proteins provided herein comprise one or more features that improve the base editing activity (e.g., efficiency, selectivity, and specificity) of the fusion proteins. For example, the fusion proteins provided herein can comprise a Cas9 domain that has reduced nuclease activity. In some embodiments, the fusion proteins provided herein can have a Cas9 domain that does not have nuclease activity (dCas9), or a Cas9 domain that cuts one strand of a duplexed DNA molecule, referred to as a Cas9 nickase (nCas9). Without wishing to be bound by any particular theory, the presence of the catalytic residue (e.g., H840) maintains the activity of the Cas9 to cleave the non-edited (e.g., non-deaminated) strand containing a T opposite the targeted A. Mutation of the catalytic residue (e.g., D10 to A10) of Cas9 prevents cleavage of the edited strand containing the targeted A residue. Such Cas9 variants are able to generate a single-strand DNA break (nick) at a specific location based on the gRNA-defined target sequence, leading to repair of the non-edited strand, ultimately resulting in a T to C change on the non-edited strand. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In certain embodiments, a base editor comprising an adenosine deaminase can deaminate a target A of a polynucleotide comprising RNA. For example, the base editor can comprise an adenosine deaminase domain capable of deaminating a target A of an RNA polynucleotide and/or a DNA-RNA hybrid polynucleotide. In an embodiment, an adenosine deaminase incorporated into a base editor comprises all or a portion of adenosine deaminase acting on RNA (ADAR, e.g., ADAR1 or ADAR2). In another embodiment, an adenosine deaminase incorporated into a base editor comprises all or a portion of adenosine deaminase acting on tRNA (ADAT). A base editor comprising an adenosine deaminase domain can also be capable of deaminating an A nucleobase of a DNA polynucleotide. In an embodiment an adenosine deaminase domain of a base editor comprises all or a portion of an ADAT comprising one or more mutations which permit the ADAT to deaminate a target A in DNA.

The adenosine deaminase can be derived from any suitable organism (e.g., E. coli). In some embodiments, the adenine deaminase is a naturally-occurring adenosine deaminase that includes one or more mutations corresponding to any of the mutations provided herein (e.g., mutations made in any one of SEQ NOs: 1, 4-12, 14-17, 24-31, and 200-219). The corresponding residue in any homologous protein can be identified by e.g., sequence alignment and determination of homologous residues. The mutations in any naturally-occurring adenosine deaminase that corresponds to any of the mutations described herein can be generated accordingly.

### Adenosine Deaminases

Some aspects of the disclosure provide adenosine deaminases. In some embodiments, the adenosine deaminases provided herein are capable of deaminating adenine. In some embodiments, the adenosine deaminases provided herein are capable of deaminating adenine in a deoxyadenosine residue of DNA. As referred to herein, the term “adenosine deaminase” refers to a deaminase that can deaminate adenine in a deoxyadenosine residue of DNA, and in some cases, cytosine in a deoxyadenosine residue of DNA. In some embodiments, the adenosine deaminases provided herein are capable of deaminating cytosine. In some embodiments, the adenosine deaminases provided herein are capable of deaminating cytosine in a deoxyadenosine residue of DNA. The adenosine deaminase can be derived from any suitable organism (e.g., E. coli). In some embodiments, the adenine deaminase is a naturally-occurring adenosine deaminase that includes one or more mutations corresponding to any of the mutations provided herein. One of skill in the art will be able to identify the corresponding residue in any homologous protein and in the respective encoding nucleic acid by methods well known in the art, e.g., by sequence alignment and determination of homologous residues. Accordingly, one of skill in the art would be able to generate mutations in any naturally occurring adenosine deaminase that corresponds to any of the mutations described herein. In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus injluenzae, Caulobacter crescentus, or Bacillus subtilis. In some embodiments, the adenosine deaminase is from E. coli.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or 100% identical to any one of the amino acid sequences set forth in any one of SEQ ID NOs: 1, 4-12, 14-17, 24-31, 36, 39-47, 49-52, 59, 71, 74-82, 84-87, 94, 116-140, and 200-242 or to any of the adenosine deaminases provided herein. It should be appreciated that adenosine deaminases provided herein can include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the amino acid sequences set forth in SEQ ID NOs: 1, 4-12, 14-17, 24-31, 200-219 or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences set forth in SEQ ID NOs: 1, 4-12, 14-17, 24-31, 36, 39-47, 49-52, 59, 71, 74-82, 84-87, 94, 116-140, 200-242 or any of the adenosine deaminases provided herein.

In some embodiments, the deaminases provided herein are capable of deaminating adenine. In some embodiments, the adenosine deaminases provided herein are capable of deaminating adenine in a deoxyadenosine residue of DNA.

In some embodiments, the deaminases provided herein are capable of deaminating cytosine or 5-methylcytosine to uracil or thymine. In some embodiments, the deaminases provided herein are capable of deaminating cytosine in DNA.

In some embodiments, the adenosine deaminase comprises one or more amino acid substitutions selected from the group consisting of W45R, H58L, P70A, L106F, A128V, D130N, H145Y, S168C, R174P, E177V, I178F, and K179N as numbered relative to SEQ ID NO: 1. In an embodiment, the adenosine deaminase is an adenosine deaminase enzyme having an amino acid sequence of SEQ ID NO: 36.

In some embodiments, the adenosine deaminase comprises one or more amino acid substitutions selected from the group consisting of W36R, P61A, R64L, L97F, A119V, D121N, H136Y, S159C, D160Y, R165P, and K170N as numbered relative to SEQ ID NO: 4. In an embodiment, the adenosine deaminase is an adenosine deaminase enzyme having an amino acid sequence of SEQ ID NO: 39.

In some embodiments, the adenosine deaminase comprises one or more amino acid substitutions selected from the group consisting of W23R, P48A, R51L, L84F, A106V, D108N, H123Y, S146C, D147Y, R152P, E155V, and K157N as numbered relative to SEQ ID NO: 5. In an embodiment, the adenosine deaminase is an adenosine deaminase enzyme having an amino acid sequence of SEQ ID NO: 40.

In some embodiments, the adenosine deaminase comprises one or more amino acid substitutions selected from the group consisting of W23R, P48A, R51L, L84F, A106V, D108N, H123Y, S146C, R152P, E155V, and K157N as numbered relative to SEQ ID NO: 6. In an embodiment, the adenosine deaminase is an adenosine deaminase enzyme having an amino acid sequence of SEQ ID NO: 41.

In some embodiments, the adenosine deaminase comprises one or more amino acid substitutions selected from the group consisting of W22R, P47A, R50L, L83F, A105V, D107N, H122Y, S145C, D146Y, R151P and K156N as numbered relative to SEQ ID NO: 7. In an embodiment, the adenosine deaminase is an adenosine deaminase enzyme having an amino acid sequence of SEQ ID NO: 42.

In some embodiments, the adenosine deaminase comprises one or more amino acid substitutions selected from the group consisting of W23R, L84F, A106V, D108N, H123Y, S146C, D147Y, R152P, E155V, and K157N as numbered relative to SEQ ID NO: 8. In an embodiment, the adenosine deaminase is an adenosine deaminase enzyme having an amino acid sequence of SEQ ID NO: 43.

In some embodiments, the adenosine deaminase comprises one or more amino acid substitutions selected from the group consisting of W21R, P46A, R49L, L82F, A104V, D106N, H121Y, S144C, D145Y, R150P, and K155N as numbered relative to SEQ ID NO: 9. In an embodiment, the adenosine deaminase is an adenosine deaminase enzyme having an amino acid sequence of SEQ ID NO: 44.

In some embodiments, the adenosine deaminase comprises one or more amino acid substitutions selected from the group consisting of W21R, H34L, P46A, L82F, A104V, D106N, H121Y, S144C, D145Y, R150P, E153V, and K155N as numbered relative to SEQ ID NO: 10. In an embodiment, the adenosine deaminase is an adenosine deaminase enzyme having an amino acid sequence of SEQ ID NO: 45.

In some embodiments, the adenosine deaminase comprises one or more amino acid substitutions selected from the group consisting of W21R, P46A, L82F, A104V, D106N, H121Y, S144C, D145Y, R150P, E153V, and K155N as numbered relative to SEQ ID NO: 11. In an embodiment, the adenosine deaminase is an adenosine deaminase enzyme having an amino acid sequence of SEQ ID NO: 46.

In some embodiments, the adenosine deaminase comprises one or more amino acid substitutions selected from the group consisting of W21R, H34L, P46A, L82F, A104V, D106N, H121Y, S144C, D145Y, R150P, E153V, and K155N as numbered relative to SEQ ID NO: 12. In an embodiment, the adenosine deaminase is an adenosine deaminase enzyme having an amino acid sequence of SEQ ID NO: 47.

In some embodiments, the adenosine deaminase comprises one or more amino acid substitutions selected from the group consisting of W28R, P53A, L89F, A111V, D113N, H128Y, S151C, D152Y, R157P, and K162N as numbered relative to SEQ ID NO: 14. In an embodiment, the adenosine deaminase is an adenosine deaminase enzyme having an amino acid sequence of SEQ ID NO: 49.

In some embodiments, the adenosine deaminase comprises one or more amino acid substitutions selected from the group consisting of W21R, P46A, L82F, A104V, D106N, H121Y, S144C, D145Y, R150P and K155N as numbered relative to SEQ ID NO: 15. In an embodiment, the adenosine deaminase is an adenosine deaminase enzyme having an amino acid sequence of SEQ ID NO: 50.

In some embodiments, the adenosine deaminase comprises one or more amino acid substitutions selected from the group consisting of W21R, P46A, L82F, A104V, D106N, H121Y, S144C, D145Y, R150P and K155N as numbered relative to SEQ ID NO: 16. In an embodiment, the adenosine deaminase is an adenosine deaminase enzyme having an amino acid sequence of SEQ ID NO: 51.

In some embodiments, the adenosine deaminase comprises one or more amino acid substitutions selected from the group consisting of W21R, P46A, L82F, A104V, D106N, H121Y, S144C, D145Y, R150P and K155N as numbered relative to SEQ ID NO: 17. In an embodiment, the adenosine deaminase is an adenosine deaminase enzyme having an amino acid sequence of SEQ ID NO: 52.

In some embodiments, the adenosine deaminase comprises one or more amino acid substitutions selected from the group consisting of L125F, A147V, D149N, H164Y, S187C, R193P, E196V, and K198N as numbered relative to SEQ ID NO: 24. In an embodiment, the adenosine deaminase is an adenosine deaminase enzyme having an amino acid sequence of SEQ ID NO: 59.

In some embodiments, the adenosine deaminase comprises one or more amino acid substitutions selected from the group consisting of W45R, H58L, P70A, L106F, A128V, D130N, A131S, T133R, D141N, H144N, H145Y, S168C, F171Y, R174P, E177V, I178F, K179N, T188I, and D189N as numbered relative to SEQ ID NO: 1. In an embodiment, the adenosine deaminase is an adenosine deaminase enzyme having an amino acid sequence of SEQ ID NO: 71.

In some embodiments, the adenosine deaminase comprises one or more amino acid substitutions selected from the group consisting of W36R, P61A, R64L, L97F, A119V, D121N, A122S, T124R, D132N, H135N, H136Y, S159C, F162Y, R165P, K170N, and D183N as numbered relative to SEQ ID NO: 4. In an embodiment, the adenosine deaminase is an adenosine deaminase enzyme having an amino acid sequence of SEQ ID NO: 74.

In some embodiments, the adenosine deaminase comprises one or more amino acid substitutions selected from the group consisting of W23R, P48A, R51L, L84F, A106V, D108N, A109S, T111R, H123Y, S146C, F149Y, R152P, E155V, K157N, and D166N as numbered relative to SEQ ID NO: 5. In an embodiment, the adenosine deaminase is an adenosine deaminase enzyme having an amino acid sequence of SEQ ID NO: 75.

In some embodiments, the adenosine deaminase comprises one or more amino acid substitutions selected from the group consisting of W23R, P48A, R51L, L84F, A106V, D108N, A109S, T111R, H123Y, S146C, F149Y, R152P, E155V, K157N, and D166N as numbered relative to SEQ ID NO: 6. In an embodiment, the adenosine deaminase is an adenosine deaminase enzyme having an amino acid sequence of SEQ ID NO: 76.

In some embodiments, the adenosine deaminase comprises one or more amino acid substitutions selected from the group consisting of W22R, P47A, R50L, L83F, A105V, D107N, A108S, T110R, D118N, H122Y, S145C, F148Y, R151P and K156N as numbered relative to SEQ ID NO: 7. In an embodiment, the adenosine deaminase is an adenosine deaminase enzyme having an amino acid sequence of SEQ ID NO: 77.

In some embodiments, the adenosine deaminase comprises one or more amino acid substitutions selected from the group consisting of W23R, L84F, A106V, D108N, A109S, T111R, D119N, H123Y, S146C, F149Y, R152P, E155V, and K157N as numbered relative to SEQ ID NO: 8. In an embodiment, the adenosine deaminase is an adenosine deaminase enzyme having an amino acid sequence of SEQ ID NO: 78.

In some embodiments, the adenosine deaminase comprises one or more amino acid substitutions selected from the group consisting of W21R, P46A, R49L, L82F, A104V, D106N, A107S, T109R, D117N, H121Y, S144C, F147Y, R150P, K155N, and D165N as numbered relative to SEQ ID NO: 9. In an embodiment, the adenosine deaminase is an adenosine deaminase enzyme having an amino acid sequence of SEQ ID NO: 79.

In some embodiments, the adenosine deaminase comprises one or more amino acid substitutions selected from the group consisting of W21R, H34L, P46A, L82F, A104V, D106N, A107S, T109R, D117N, H121Y, S144C, F147Y, R150P, E153V, and K155N as numbered relative to SEQ ID NO: 10. In an embodiment, the adenosine deaminase is an adenosine deaminase enzyme having an amino acid sequence of SEQ ID NO: 80.

In some embodiments, the adenosine deaminase comprises one or more amino acid substitutions selected from the group consisting of W21R, P46A, L82F, A104V, D106N, T109R, D117N, H121Y, S144C, F147Y, R150P, E153V, K155N, and D165N as numbered relative to SEQ ID NO: 11. In an embodiment, the adenosine deaminase is an adenosine deaminase enzyme having an amino acid sequence of SEQ ID NO: 81.

In some embodiments, the adenosine deaminase comprises one or more amino acid substitutions selected from the group consisting of W21R, H34L, P46A, L82F, A104V, D106N, T109R, D117N, H121Y, S144C, F147Y, R150P, E153V, and K155N as numbered relative to SEQ ID NO: 12. In an embodiment, the adenosine deaminase is an adenosine deaminase enzyme having an amino acid sequence of SEQ ID NO: 82.

In some embodiments, the adenosine deaminase comprises one or more amino acid substitutions selected from the group consisting of W28R, P53A, L89F, A111V, D113N, T116R, D124N, H128Y, S151C, F154Y, R157P, and K162N as numbered relative to SEQ ID NO: 14. In an embodiment, the adenosine deaminase is an adenosine deaminase enzyme having an amino acid sequence of SEQ ID NO: 84.

In some embodiments, the adenosine deaminase comprises one or more amino acid substitutions selected from the group consisting of W21R, P46A, L82F, A104V, D106N, T109R, D117N, H121Y, S144C, F147Y, R150P, K155N, and T163I as numbered relative to SEQ ID NO: 15. In an embodiment, the adenosine deaminase is an adenosine deaminase enzyme having an amino acid sequence of SEQ ID NO: 85.

In some embodiments, the adenosine deaminase comprises one or more amino acid substitutions selected from the group consisting of W21R, P46A, L82F, A104V, D106N, T109R, D117N, H121Y, S144C, F147Y, R150P and K155N as numbered relative to SEQ ID NO: 16. In an embodiment, the adenosine deaminase is an adenosine deaminase enzyme having an amino acid sequence of SEQ ID NO: 86.

In some embodiments, the adenosine deaminase comprises one or more amino acid substitutions selected from the group consisting of W21R, P46A, L82F, A104V, D106N, T109R, D117N, H121Y, S144C, F147Y, R150P and K155N as numbered relative to SEQ ID NO: 17. In an embodiment, the adenosine deaminase is an adenosine deaminase enzyme having an amino acid sequence of SEQ ID NO: 87.

In some embodiments, the adenosine deaminase comprises one or more amino acid substitutions selected from the group consisting of L125F, A147V, D149N, T152R, H164Y, S187C, F190Y, R193P, E196V, K198N, and T210I as numbered relative to SEQ ID NO: 24. In an embodiment, the adenosine deaminase is an adenosine deaminase enzyme having an amino acid sequence of SEQ ID NO: 94.

In some embodiments, the adenosine deaminase comprises one or more amino acid substitutions selected from the group consisting of W23R, P48A, L84F, A106V, D108N, T111R, D119N, H123Y, S146C, F149Y, R152P, K157N, and D169N as numbered relative to SEQ ID NO: 25. In an embodiment, the adenosine deaminase is an adenosine deaminase enzyme having an amino acid sequence of SEQ ID NO: 116.

In some embodiments, the adenosine deaminase comprises one or more amino acid substitutions selected from the group consisting of W23R, P48A, L84F, A106V, D108N, T111R, D119N, H123Y, S146C, F149Y, R152P, K157N, and D169N as numbered relative to SEQ ID NO: 26. In an embodiment, the adenosine deaminase is an adenosine deaminase enzyme having an amino acid sequence of SEQ ID NO: 117.

In some embodiments, the adenosine deaminase comprises one or more amino acid substitutions selected from the group consisting of W22R, P47A, L83F, A105V, D107N, T10R, D118N, H122Y, S145C, F148Y, R151P, E154V, and K156N as numbered relative to SEQ ID NO: 27. In an embodiment, the adenosine deaminase is an adenosine deaminase enzyme having an amino acid sequence of SEQ ID NO: 118.

In some embodiments, the adenosine deaminase comprises one or more amino acid substitutions selected from the group consisting of W21R, P46A, L82F, A104V, D106N, T109R, D117N, H121Y, S144C, F147Y, R150P, and K155N as numbered relative to SEQ ID NO: 28. In an embodiment, the adenosine deaminase is an adenosine deaminase enzyme having an amino acid sequence of SEQ ID NO: 119.

In some embodiments, the adenosine deaminase comprises one or more amino acid substitutions selected from the group consisting of W21R, P46A, L82F, A104V, D106N, A107S, T109R, D117N, H121Y, S144C, F147Y, R150P, E153V, and K155N as numbered relative to SEQ ID NO: 29. In an embodiment, the adenosine deaminase is an adenosine deaminase enzyme having an amino acid sequence of SEQ ID NO: 120.

In some embodiments, the adenosine deaminase comprises one or more amino acid substitutions selected from the group consisting of W21R, H34L, P46A, L82F, A104V, D106N, T109R, D117N, H121Y, S144C, F147Y, R150P, E153V, and K155N as numbered relative to SEQ ID NO: 30. In an embodiment, the adenosine deaminase is an adenosine deaminase enzyme having an amino acid sequence of SEQ ID NO: 121.

In some embodiments, the adenosine deaminase comprises one or more amino acid substitutions selected from the group consisting of W21R, P46A, L82F, A104V, D106N, A107S, T109R, D117N, H121Y, S144C, F147Y, R150P, and K155N as numbered relative to SEQ ID NO: 31. In an embodiment, the adenosine deaminase is an adenosine deaminase enzyme having an amino acid sequence of SEQ ID NO: 122.

In some embodiments, the adenosine deaminase comprises one or more amino acid substitutions selected from the group consisting of W45R, H58L, P70A, L106F, A128V, D130Q, A131S, T133R, D141N, H144N, H145Y, S168C, F171Y, R174P, E177V, I178F, K179N, T188I, and D189N as numbered relative to SEQ ID NO: 1. In an embodiment, the adenosine deaminase is an adenosine deaminase enzyme having an amino acid sequence of SEQ ID NO: 123.

In some embodiments, the adenosine deaminase comprises one or more amino acid substitutions selected from the group consisting of W36R, P61A, R64L, L97F, A119V, D121Q, A122S, T124R, D132N, H135N, H136Y, S159C, F162Y, R165P, K170N, and D183N as numbered relative to SEQ ID NO: 4. In an embodiment, the adenosine deaminase is an adenosine deaminase enzyme having an amino acid sequence of SEQ ID NO: 124.

In some embodiments, the adenosine deaminase comprises one or more amino acid substitutions selected from the group consisting of W45R, E49A, V50A, H58L, P70A, M831, L106F, H118N, A128V, D130N, A131S, T133R, D141N, H144N, H145Y, S168C, F171Y, R174P, E177V, I178F, K179N, T1881, and D189N as numbered relative to SEQ ID NO: 1. In an embodiment, the adenosine deaminase is an adenosine deaminase enzyme having an amino acid sequence of SEQ ID NO: 125.

In some embodiments, the adenosine deaminase comprises one or more amino acid substitutions selected from the group consisting of W45R, R48G, E49A, V50G, H58L, P70A, L98F, L106F, H118N, A128V, D130N, A131S, T133R, D141N, H144N, H145Y, S168C, F171Y, R174P, E177V, I178F, K179N, A180S, T1881, and D189N as numbered relative to SEQ ID NO: 1. In an embodiment, the adenosine deaminase is an adenosine deaminase enzyme having an amino acid sequence of SEQ ID NO: 126.

In some embodiments, the adenosine deaminase comprises one or more amino acid substitutions selected from the group consisting of W36R, E40A, V41A, P61A, R64L, M74I, L97F, H109N, A119V, D121N, A122S, T124R, D132N, H135N, H136Y, S159C, F162Y, R165P, K170N, and D183N as numbered relative to SEQ ID NO: 4. In an embodiment, the adenosine deaminase is an adenosine deaminase enzyme having an amino acid sequence of SEQ ID NO: 127.

In some embodiments, the adenosine deaminase comprises one or more amino acid substitutions selected from the group consisting of W36R, E40A, V41G, P61A, R64L, I89F, L97F, H109N, A119V, D121N, A122S, T124R, D132N, H135N, H136Y, S159C, F162Y, R165P, K170N, A171S, and D183N as numbered relative to SEQ ID NO: 4. In an embodiment, the adenosine deaminase is an adenosine deaminase enzyme having an amino acid sequence of SEQ ID NO: 128.

In some embodiments, the adenosine deaminase comprises one or more amino acid substitutions selected from the group consisting of W23R, E27A, V28A, P48A, R51L, M61I, L84F, H96N, A106V, D108N, A109S, TIIR, H123Y, S146C, F149Y, R152P, E155V, K157N, and D166N as numbered relative to SEQ ID NO: 5. In an embodiment, the adenosine deaminase is an adenosine deaminase enzyme having an amino acid sequence of SEQ ID NO: 129.

In some embodiments, the adenosine deaminase comprises one or more amino acid substitutions selected from the group consisting of W23R, E27A, V28G, P48A, R51L, L76F, L84F, H96N, A106V, D108N, A109S, T111R, H123Y, S146C, F149Y, R152P, E155V, K157N, A158S, and D166N as numbered relative to SEQ ID NO: 5. In an embodiment, the adenosine deaminase is an adenosine deaminase enzyme having an amino acid sequence of SEQ ID NO: 130.

In some embodiments, the adenosine deaminase comprises one or more amino acid substitutions selected from the group consisting of W23R, E27A, V28A, M61I, L84F, H96N, A106V, D108N, A109S, T111R, D119N, H123Y, S146C, F149Y, R152P, E155V, and K157N as numbered relative to SEQ ID NO: 8. In an embodiment, the adenosine deaminase is an adenosine deaminase enzyme having an amino acid sequence of SEQ ID NO: 131.

In some embodiments, the adenosine deaminase comprises one or more amino acid substitutions selected from the group consisting of W23R, E27A, V28G, L76F, L84F, H96N, A106V, D108N, A109S, T111R, D119N, H123Y, S146C, F149Y, R152P, E155V, K157N and A158S as numbered relative to SEQ ID NO: 8. In an embodiment, the adenosine deaminase is an adenosine deaminase enzyme having an amino acid sequence of SEQ ID NO: 132.

In some embodiments, the adenosine deaminase comprises one or more amino acid substitutions selected from the group consisting of W21R, E25A, V26A, P46A, R49L, M59I, L82F, H94N, A104V, D106N, A107S, T109R, D117N, H121Y, S144C, F147Y, R150P, K155N, and D165N as numbered relative to SEQ ID NO: 9. In an embodiment, the adenosine deaminase is an adenosine deaminase enzyme having an amino acid sequence of SEQ ID NO: 133.

In some embodiments, the adenosine deaminase comprises one or more amino acid substitutions selected from the group consisting of W21R, E25A, V26G, P46A, R49L, L74F, L82F, H94N, A104V, D106N, A107S, T109R, D117N, H121Y, S144C, F147Y, R150P, K155N, A156S, and D165N as numbered relative to SEQ ID NO: 9. In an embodiment, the adenosine deaminase is an adenosine deaminase enzyme having an amino acid sequence of SEQ ID NO: 134.

In some embodiments, the adenosine deaminase comprises one or more amino acid substitutions selected from the group consisting of W21R, E25A, V26A, H34L, P46A, M59I, L82F, H94N, A104V, D106N, A107S, T109R, D117N, H121Y, S144C, F147Y, R150P, E153V, and K155N as numbered relative to SEQ ID NO: 10. In an embodiment, the adenosine deaminase is an adenosine deaminase enzyme having an amino acid sequence of SEQ ID NO: 135.

In some embodiments, the adenosine deaminase comprises one or more amino acid substitutions selected from the group consisting of W21R, E25A, V26G, H34L, P46A, L74F, L82F, H94N, A104V, D106N, A107S, T109R, D117N, H121Y, S144C, F147Y, R150P, E153V, K155N, and A156S as numbered relative to SEQ ID NO: 10. In an embodiment, the adenosine deaminase is an adenosine deaminase enzyme having an amino acid sequence of SEQ ID NO: 136.

In some embodiments, the adenosine deaminase comprises one or more amino acid substitutions selected from the group consisting of W21R, E25A, V26A, H34L, P46A, M59I, L82F, H94N, A104V, D106N, T109R, D117N, H121Y, S144C, F147Y, R150P, E153V, and K155N as numbered relative to SEQ ID NO: 12. In an embodiment, the adenosine deaminase is an adenosine deaminase enzyme having an amino acid sequence of SEQ ID NO: 137.

In some embodiments, the adenosine deaminase comprises one or more amino acid substitutions selected from the group consisting of W21R, E25A, V26G, H34L, P46A, L74F, L82F, H94N, A104V, D106N, T109R, D117N, H121Y, S144C, F147Y, R150P, E153V, K155N, and A156S as numbered relative to SEQ ID NO: 12. In an embodiment, the adenosine deaminase is an adenosine deaminase enzyme having an amino acid sequence of SEQ ID NO: 138.

In some embodiments, the adenosine deaminase comprises one or more amino acid substitutions selected from the group consisting of E68A, V69A, L125F, H137N, A147V, D149N, T152R, H164Y, S187C, F190Y, R193P, E196V, K198N, and T210I as numbered relative to SEQ ID NO: 24. In an embodiment, the adenosine deaminase is an adenosine deaminase enzyme having an amino acid sequence of SEQ ID NO: 139.

In some embodiments, the adenosine deaminase comprises one or more amino acid substitutions selected from the group consisting of E68A, V69G, L117F, L125F, H137N, A147V, D149N, T152R, H164Y, S187C, F190Y, R193P, E196V, K198N, A199S, and T2101 as numbered relative to SEQ ID NO: 24. In an embodiment, the adenosine deaminase is an adenosine deaminase enzyme having an amino acid sequence of SEQ ID NO: 140.

In some embodiments, the adenosine deaminase comprises one or more amino acid substitutions selected from the group consisting of L120F, A142V, D144N, T147R, H159Y, S182C, F185Y, R188P, E191V, and K193N as numbered relative to SEQ ID NO: 202. In an embodiment, the adenosine deaminase is an adenosine deaminase enzyme having an amino acid sequence of SEQ ID NO: 220.

In some embodiments, the adenosine deaminase comprises one or more amino acid substitutions selected from the group consisting of L121F, A143V, D145N, T148R, H160Y, S183C, F186Y, R189P, E192V, and K194N as numbered relative to SEQ ID NO: 203. In an embodiment, the adenosine deaminase is an adenosine deaminase enzyme having an amino acid sequence of SEQ ID NO: 221.

In some embodiments, the adenosine deaminase comprises one or more amino acid substitutions selected from the group consisting of W21R, P46A, L82F, A104V, D106N, T109R, D117N, H121Y, S144C, F147Y, R150P, and K155N as numbered relative to SEQ ID NO: 205. In an embodiment, the adenosine deaminase is an adenosine deaminase enzyme having an amino acid sequence of SEQ ID NO: 222.

In some embodiments, the adenosine deaminase comprises one or more amino acid substitutions selected from the group consisting of L79F, A101V, D103N, T106R, H118Y, S141C, F144Y, R147P, E150V, K152N, and D162N as numbered relative to SEQ ID NO: 206. In an embodiment, the adenosine deaminase is an adenosine deaminase enzyme having an amino acid sequence of SEQ ID NO: 223.

In some embodiments, the adenosine deaminase comprises one or more amino acid substitutions selected from the group consisting of W41R, P66A, L102F, A124V, D126N, A127S, T129R, D137N, H141Y, S164C, F167Y, R170P, E173V, and K175N as numbered relative to SEQ ID NO: 209. In an embodiment, the adenosine deaminase is an adenosine deaminase enzyme having an amino acid sequence of SEQ ID NO: 224.

In some embodiments, the adenosine deaminase comprises one or more amino acid substitutions selected from the group consisting of P52A, L88F, A110V, D112N, T115R, D123N, H127Y, S150C, F153Y, R156P, and K161N as numbered relative to SEQ ID NO: 210. In an embodiment, the adenosine deaminase is an adenosine deaminase enzyme having an amino acid sequence of SEQ ID NO: 225.

In some embodiments, the adenosine deaminase comprises one or more amino acid substitutions selected from the group consisting of W21R, P46A, L82F, A104V, D106N, T109R, D117N, H121Y, S144C, F147Y, R150P, K155N and D165N as numbered relative to SEQ ID NO: 211. In an embodiment, the adenosine deaminase is an adenosine deaminase enzyme having an amino acid sequence of SEQ ID NO: 226.

In some embodiments, the adenosine deaminase comprises one or more amino acid substitutions selected from the group consisting of P57A, L93F, A115V, D117N, T120R, D128N, H132Y, S155C, F158Y, and R161P as numbered relative to SEQ ID NO: 212. In an embodiment, the adenosine deaminase is an adenosine deaminase enzyme having an amino acid sequence of SEQ ID NO: 227.

In some embodiments, the adenosine deaminase comprises one or more amino acid substitutions selected from the group consisting of W22R, P47A, L83F, A105V, D107N, A108S, T10R, D118N, H122Y, S145C, F148Y, R151P, and K156N as numbered relative to SEQ ID NO: 215. In an embodiment, the adenosine deaminase is an adenosine deaminase enzyme having an amino acid sequence of SEQ ID NO: 228.

In some embodiments, the adenosine deaminase comprises one or more amino acid substitutions selected from the group consisting of W22R, L83F, A105V, D107N, A108S, T10R, D118N, H122Y, S145C, F148Y, R151P, E154V and K156N as numbered relative to SEQ ID NO: 216. In an embodiment, the adenosine deaminase is an adenosine deaminase enzyme having an amino acid sequence of SEQ ID NO: 229.

In some embodiments, the adenosine deaminase comprises one or more amino acid substitutions selected from the group consisting of W21R, H34L, P46A, L82F, A104V, D106N, A107S, T109R, D117N, H121Y, S144C, F147Y, R150P, E153V, and K155N as numbered relative to SEQ ID NO: 217. In an embodiment, the adenosine deaminase is an adenosine deaminase enzyme having an amino acid sequence of SEQ ID NO: 230.

In some embodiments, the adenosine deaminase comprises one or more amino acid substitutions selected from the group consisting of W21R, P46A, L82F, A104V, D106N, A107S, T109R, D117N, H121Y, S144C, F147Y, R150P, and K155N as numbered relative to SEQ ID NO: 218. In an embodiment, the adenosine deaminase is an adenosine deaminase enzyme having an amino acid sequence of SEQ ID NO: 231.

In some embodiments, the adenosine deaminase comprises one or more amino acid substitutions selected from the group consisting of W21R, H34L, P46A, L82F, A104V, D106N, T109R, H121Y, S144C, F147Y, R150P, and K155N as numbered relative to SEQ ID NO: 219. In an embodiment, the adenosine deaminase is an adenosine deaminase enzyme having an amino acid sequence of SEQ ID NO: 232.

In some embodiments, the adenosine deaminase comprises one or more amino acid substitutions selected from the group consisting of W20R, P45A, L81F, D105N, S143C, D144R, R149P, E151R, and K154N as numbered relative to SEQ ID NO: 200. In an embodiment, the adenosine deaminase is an adenosine deaminase enzyme having an amino acid sequence of SEQ ID NO: 233.

In some embodiments, the adenosine deaminase comprises one or more amino acid substitutions selected from the group consisting of P83A, L119F, F139V, D143N, S181C, D182R, R187P, K189R, E190V, I191F, and K192N as numbered relative to SEQ ID NO: 201. In an embodiment, the adenosine deaminase is an adenosine deaminase enzyme having an amino acid sequence of SEQ ID NO: 234.

In some embodiments, the adenosine deaminase comprises one or more amino acid substitutions selected from the group consisting of L125F, F145V, D149N, S187C, R193P, E195R, E196V, and K198N as numbered relative to SEQ ID NO: 24. In an embodiment, the adenosine deaminase is an adenosine deaminase enzyme having an amino acid sequence of SEQ ID NO: 235.

In some embodiments, the adenosine deaminase comprises one or more amino acid substitutions selected from the group consisting of L121F, F141V, D145N, S183C, R189P, E191R, E192V, and K194N as numbered relative to SEQ ID NO: 204. In an embodiment, the adenosine deaminase is an adenosine deaminase enzyme having an amino acid sequence of SEQ ID NO: 236.

In some embodiments, the adenosine deaminase comprises one or more amino acid substitutions selected from the group consisting of W21R, P46A, L82F, D106N, S144C, D145R, R150P, E152R, and K155N as numbered relative to SEQ ID NO: 205. In an embodiment, the adenosine deaminase is an adenosine deaminase enzyme having an amino acid sequence of SEQ ID NO: 237.

In some embodiments, the adenosine deaminase comprises one or more amino acid substitutions selected from the group consisting of L79F, F99V, D103N, S141C, G142R, R147P, A149R, E150V, and K152N as numbered relative to SEQ ID NO: 206. In an embodiment, the adenosine deaminase is an adenosine deaminase enzyme having an amino acid sequence of SEQ ID NO: 238.

In some embodiments, the adenosine deaminase comprises one or more amino acid substitutions selected from the group consisting of P46A, L82F, D106N, S144C, A145R, R150P, Q152R, E153V, and K158N as numbered relative to SEQ ID NO: 207. In an embodiment, the adenosine deaminase is an adenosine deaminase enzyme having an amino acid sequence of SEQ ID NO: 239.

In some embodiments, the adenosine deaminase comprises one or more amino acid substitutions selected from the group consisting of W20R, P45A, L81F, D105N, S143C, D144R, R149P, E151R, E152V, and K154N as numbered relative to SEQ ID NO: 208. In an embodiment, the adenosine deaminase is an adenosine deaminase enzyme having an amino acid sequence of SEQ ID NO: 240.

In some embodiments, the adenosine deaminase comprises one or more amino acid substitutions selected from the group consisting of L81F, D105N, T120H, S143C, D144R, R149P, A151R, E152V, and K154N as numbered relative to SEQ ID NO: 213. In an embodiment, the adenosine deaminase is an adenosine deaminase enzyme having an amino acid sequence of SEQ ID NO: 241.

In some embodiments, the adenosine deaminase comprises one or more amino acid substitutions selected from the group consisting of P83A, L119F, F139V, D143N, S181C, D182R, R187P, K189R, E190V, I191F, and K192N as numbered relative to SEQ ID NO: 214. In an embodiment, the adenosine deaminase is an adenosine deaminase enzyme having an amino acid sequence of SEQ ID NO: 242.

It should be appreciated that any of the mutations provided herein (e.g., relative to the amino acid sequence of SEQ ID NOs: 1, 4-12, 14-17, 24-31, 200-219) can be introduced into other adenosine deaminases, such as S. aureus TadA (saTadA), or other adenosine deaminases (e.g., bacterial adenosine deaminases). It would be apparent to the skilled artisan how to identify amino acid residues from other adenosine deaminases that are homologous to the mutated residues in SEQ ID NOs: 1, 4-12, 14-17, 24-31, 200-219. Thus, any of the mutations identified in SEQ ID NOs: 1, 4-12, 14-17, 24-31, 200-219 to produce SEQ ID NOs: 36, 39-47, 49-52, 59 and SEQ ID NOs: 71, 74-82, 84-87, 94, 116-140, and 220-242 can be made in other adenosine deaminases. It should also be appreciated that any of the mutations provided herein can be made individually or in any combination in another adenosine deaminase.

TABLE 1
Amino Acid Sequences of Deaminases

| | SEQ | |
|---|---|---|
| Name | ID NO | Amino acid sequence |
| Traw.1 | 1 | MPVQLAPGRVYNAPAFITGVTPLSDIQLSHEYWMRHALTLAK |
| | | RAWDAREVPVGAVLVHNNRAIGEGWNRPIGHHDPTAHAEIMA |
| | | LRQGGLVLENYRLLDATLYVTLEPCVMCAGAMVHSRIARVVE |
| | | GARDAKTGAAGSLMDVLHHPGMNHRVEVTEGVLGEECAALLS |
| | | EFFRMRRREIKALKRASAQTDEGQSAAAGPGER |
| Traw.4 | 4 | MYNAPRFSTGVDALSETELNHEYWMRHALNLAQRAWEEGEVP |
| | | VGAVLVYQDKVIGEGWNRPIGRHDPTAHAEIMALRQGGMVLQ |
| | | NYRLIDATLYVTLEPCVMCAGAMIHSRIGRVVFGARDAKTGA |
| | | AGSLIDVLHHPGMNHRVEVTEGVLAESCSSLLSDFFRERREQ |
| | | KKALKRASQDPRSSDA |
| Traw.5 | 5 | MSDTEFTHEYWMQHALTLAQRAWDEGEVPVGAVLVQDNRVIG |
| | | EGWNRPIGRHDPTAHAEIMALRQGGMVLQNYRLLNTTLYVTL |
| | | EPCIMCAGAMVHSRIGTLVFGARDAKTGAVGSLMNVPAHPGM |
| | | NHHMQVIEGVLAPSCSALLSDFFRVRRLEKKAQKEASRKD |
| Traw.6 | 6 | MSETEFSHEYWMRHALTLAQRAWDEGEVPVGAVLVQDNRVIG |
| | | EGWNRPIGRHDPTAHAEIMALRQGGMVLQNYRLLNTTLYVTL |
| | | EPCIMCAGAMVHSRIGTLVFGARDAKTGAVGSLMNIPAHPQM |
| | | NHHMQIIEGVLAPTCSALLSEFFRVRRLEKKAQKEALRKD |
| Traw.7 | 7 | MSDNHDHEYWMQHALTLARRAWDEGEVPVGAVLVRNGELIGE |
| | | GWNRPIGRHDPTAHAEIMALRQGGVVLQNYRLLDATLYVTLE |
| | | PCVMCAGAMVHSRIGRLVEGARDAKTGAAGSLMDVLAHPGMN |
| | | HRVEVTEGVLAPACSTLLSDEFRERRLQQKALKEASRKPE |
| Traw.8 | 8 | MTTPALTHEYWMNYALTLARRAWDEGEVPVGAVLVYDNRVIG |
| | | EGWNRSIGKHDPTAHAEIMALRQGGMVQQNYRLLDTTLYVTL |
| | | EPCVMCAGAMIHSRIGTLVFGARDAKTGAVGSQMDILNHPGM |
| | | NHQVQIIEGVLAPQCSALLSDFFRMRRKEKKALKIASRDPHP |
| | | AAPQR |
| Traw.9 | 9 | MDNHDHEYWMQHALTLAQRAWEEGEVPVGAVLVLNGQAIGEG |
| | | WNRPIGRHDPTAHAEMMALRQGGAVLQNYRLLNATLYVTLEP |
| | | CVMCAGAMVHSRIARLVEGARDAKTGAAGSLLDVLAHPGMNH |
| | | RVAVTEGVLAPACSALLSDFFRQRRQQQKALKDANRKRD |
| Traw.10 | 10 | MSDNNDEFWMRHALTLARRAWQEGEVPVGAVLVHEGRVIGEG |
| | | WNRPIGHHDPTAHAEIMALRQGGKVIENYRLLDTTLYVTLEP |
| | | CVMCAGAMVHGRVGRLVFGARDAKTGAAGSLIDVLSHPGMNH |
| | | QVQVDEGVLAEECAAMLSDFFRQRRAEKKALRQQQQQGTSSA |
| | | E |
| Traw.11 | 11 | MTDYNDELWMRHALTLARRAWDEGEVPVGAVLVQDNRVIGEG |
| | | WNRPIGHHDPTAHAEMMALRQGGKVLENYRLLDTTLYVTLEP |
| | | CVMCAGAIVHSRIGRLVFGARDGKTGAAGSLIDILGHPGMNH |
| | | QVIVSEGILAETCAGMLSDFFRQRREEKKALRKTRETPDR |
| Traw.12 | 12 | MSDNNDEYWMRHALMLARRAWDEGEVPVGAVLVHEGRVIGEG |
| | | WNRPIGHHDPTAHAEIMALRQGGKVIENYRLLDTTLYVTLEP |
| | | CVMCAGAMVHSRIGRLVFGARDEKTGAAGSLLDVLGHPGMNH |
| | | QVQIEEGILAAECAAMLSDFFRHRRAEKKALRQAGKLL |
| Traw.14 | 14 | MLPGVVCVTQEQDEYWMRRALTLAQRAWEQGEVPVGAVLVQG |
| | | DRVIGEGWNRPIGQHDPTAHAEIMALRQGGKVLENYRLLNTT |
| | | LYVTLEPCIMCAGAMVHSRIGRLVYGAHDVKTGAAGSLIDIL |
| | | GHPGMNHQVALHQGVLEEECAAMLSDFFRMRRQQQKALRQAQ |
| | | RES |
| Traw.15 | 15 | MNPQTDEYWMRYALELAKRAWEQGEVPVGAVLVQGDKVIGEG |
| | | WNRPIGQHDPTAHAEIMALRQGGKVLENYRLLDTTLYVTLEP |
| | | CVMCAGAMVHGRITRLVYGAKDDKTGAAGSLLDVIGHPGMNH |
| | | QIQIDCGVLAEECAGMLSDFFRMRREQKKALRQAQRTG |
| Traw.16 | 16 | MNQQRDEYWMRHALGLARLAWEQGEVPVGAVLVQDERVIGEG |
| | | WNRPIGQHDPTAHAEMMALRQGGKVLENYRLLNTTLYVTLEP |
| | | CVMCAGAMVHSRITRLVYGAHDVKTGAAGSLLDVLGHPGMNH |
| | | QIELHSGVLAEECAAMLSDFFRMRREQKKALRQAQRQS |
| Traw.17 | 17 | MNQQHDEYWMRHALGLARLAWEQGEVPVGAVLVQADRVIGEG |
| | | WNRPIGQHDPTAHAEMMALRQGGKVLENYRLLNTTLYVTLEP |
| | | CVMCAGAMVHSRITRLVYGAHDVKTGAAGSLLDVLGHPGMNH |
| | | QVELHSGVLADECAAMLSDEFQMRREQKKALRQAQRQG |
| Traw.24 | 24 | MEQIKLDPKTLHAKTNSSDELGCDEVSADVLAGEQPCLEQAS |
| | | LEQARVDEHWMRVAMTMAEKAEAAGEVPVGAVLVKDGQQIAA |
| | | GYNLSISEHDPCAHAEIQCLRAAGQTIENYRLLDTTLYVTLE |
| | | PCAMCAGAMVHSRIARVVFGAKDEKTGAAGTVLNLLQHPAFN |
| | | HQVEVTSGVLAQECADQLSRFFKRRREEKKALKQAQKAQQGT |
| | | LS |
| Traw.101 | 25 | MTESETDHIRWMRHALSLAQRAWDEGEVPVGAVLVYQGQVIG |
| | | EGWNRPIGHHDPTAHAEMMALRQGGIVLQNYRLLDTTLYVTL |
| | | EPCVMCAGAMVHSRIGQLIYGASDVKTGAAGSLMDVLGHPGM |
| | | NHKVSVAGGVLAQECAGLLSDFFRMRRQVHKANKQAARQPSE |
| | | DQ |
| Traw.102 | 26 | MTGSETDHIRWMRHALTLAQRAWDEGEVPVGAVLVYQGQVIG |
| | | EGWNRPIGHHDPTAHAEMMALRQGGIVLQNYRLLDTTLYVTL |
| | | EPCVMCAGAMVHSRIGQLIYGASDVKTGAAGSLMDVLGHPGM |
| | | NHKVSVAGGVLAQECAGLLSDFFRMRRQVHKANKQAARQQSE |
| | | DQ |
| Traw.108 | 27 | MSDTLIDEKWMRHALTLARRAWEEGEVPVGAVLVQGDTVIGE |
| | | GWNRPIGHHDPTAHAEIMALRQGGKVLENYRLLDTTLYVTLE |
| | | PCVMCAGAMVHGRVGRLVFGARDEKTGAAGSLLDILGHAGMN |
| | | HQVRVDQGVLAAECAAMLSDFFRHRRAEKKALRDRLRAERLK |
| | | GE |
| Traw.109 | 28 | MTDYNDEFWMRHALTLAQRAWDEGEVPVGAVLVQGNRVIGEG |
| | | WNRPIGHHDPTAHAEIMALRQGGKVLENYRLIDTTLYVTLEP |
| | | CVMCAGAIVHSRIGRLVFGARDEKTGAAGSLLDVLGHPGMNH |
| | | QVKMTEGILAQQCAGMLSDFFRMRREQKKALRKSVKQ |
| Traw.110 | 29 | MTDHNDEYWMRHALRLARRAWDEGEVPVGAVLVLDNQVIGEG |
| | | WNRPIGHHDPTAHAEMMALRQGGKMIENYRLLDTTLYVTLEP |
| | | CVMCAGAMIHSRIGRLVYGARDAKTGAAGSLLDVLGHPGMNH |
| | | QVPAECGLLNDECAAMLSDFFRQRRAEKKMLRQQQNPGRV |
| Traw.111 | 30 | MSDNNDEYWMRHALMLARRAWDEGEVPVGAVLVHEGRAIGEG |
| | | WNRPIGHHDPTAHAEIMALRQGGKVIENYRLLNTTLYVTLEP |
| | | CVMCAGAMVHSRIGRLVFGARDGKTGAAGSLLDVLGHPGMNH |
| | | QVQIEEGILATECAAMLSDFFRHRRAEKKAQRQAGKLL |
| Traw.113 | 31 | MTQEQDEYWMRRALTLAQRAWEQGEVPVGAVLVQGDRVIGEG |
| | | WNRPIGQHDPTAHAEIMALRQGGKVLENYRLLNTTLYVTLEP |
| | | CIMCAGAMVHSRIGRLVYGAHDAKTGAAGSLIDILGHPGMNH |
| | | QVALHQGVLEEECAAMLSDFFRMRRQQQKALRQAQRES |
| T7.1 | 36 | MPVQLAPGRVYNAPAFITGVTPLSDIQLSHEYWMRHALTLAK |
| | | RARDAREVPVGAVLVLNNRAIGEGWNRAIGHHDPTAHAEIMA |
| | | LRQGGLVLENYRLLDATLYVTFEPCVMCAGAMVHSRIARVVF |
| | | GVRNAKTGAAGSLMDVLHYPGMNHRVEVTEGVLGEECAALLC |
| | | EFFRMPRRVENALKRASAQTDEGQSAAAGPGER |
| T7.4 | 39 | MYNAPRFSTGVDALSETELNHEYWMRHALNLAQRAREEGEVP |
| | | VGAVLVYQDKVIGEGWNRAIGLHDPTAHAEIMALRQGGMVLQ |
| | | NYRLIDATLYVTFEPCVMCAGAMIHSRIGRVVFGVRNAKTGA |
| | | AGSLIDVLHYPGMNHRVEVTEGVLAESCSSLLCYFFREPREQ |
| | | KNALKRASQDPRSSDA |
| T7.5 | 40 | MSDTEFTHEYWMQHALTLAQRARDEGEVPVGAVLVQDNRVIG |
| | | EGWNRAIGLHDPTAHAEIMALRQGGMVLQNYRLLNTTLYVTF |
| | | EPCIMCAGAMVHSRIGTLVFGVRNAKTGAVGSLMNVPAYPGM |
| | | NHHMQVIEGVLAPSCSALLCYFFRVPRLVKNAQKEASRKD |
| T7.6 | 41 | MSETEFSHEYWMRHALTLAQRARDEGEVPVGAVLVQDNRVIG |
| | | EGWNRAIGLHDPTAHAEIMALRQGGMVLQNYRLLNTTLYVTF |
| | | EPCIMCAGAMVHSRIGTLVFGVRNAKTGAVGSLMNIPAYPQM |
| | | NHHMQIIEGVLAPTCSALLCEFFRVPRLVKNAQKEALRKD |
| T7.7 | 42 | MSDNHDHEYWMQHALTLARRARDEGEVPVGAVLVRNGELIGE |
| | | GWNRAIGLHDPTAHAEIMALRQGGVVLQNYRLLDATLYVTFE |
| | | PCVMCAGAMVHSRIGRLVFGVRNAKTGAAGSLMDVLAYPGMN |
| | | HRVEVTEGVLAPACSTLLCYFFREPRLQQNALKEASRKPE |
| T7.8 | 43 | MTTPALTHEYWMNYALTLARRARDEGEVPVGAVLVYDNRVIG |
| | | EGWNRSIGKHDPTAHAEIMALRQGGMVQQNYRLLDTTLYVTF |
| | | EPCVMCAGAMIHSRIGTLVFGVRNAKTGAVGSQMDILNYPGM |
| | | NHQVQIIEGVLAPQCSALLCYFFRMPRKVKNALKIASRDPHP |
| | | AAPQR |
| T7.9 | 44 | MDNHDHEYWMQHALTLAQRAREEGEVPVGAVLVLNGQAIGEG |
| | | WNRAIGLHDPTAHAEMMALRQGGAVLQNYRLLNATLYVTFEP |
| | | CVMCAGAMVHSRIARLVFGVRNAKTGAAGSLLDVLAYPGMNH |
| | | RVAVTEGVLAPACSALLCYFFRQPRQQQNALKDANRKRD |
| T7.10 | 45 | MSDNNDEFWMRHALTLARRARQEGEVPVGAVLVLEGRVIGEG |
| | | WNRAIGHHDPTAHAEIMALRQGGKVIENYRLLDTTLYVTFEP |
| | | CVMCAGAMVHGRVGRLVFGVRNAKTGAAGSLIDVLSYPGMNH |
| | | QVQVDEGVLAEECAAMLCYFFRQPRAVKNALRQQQQQGTSSA |
| T7.11 | 46 | MTDYNDELWMRHALTLARRARDEGEVPVGAVLVQDNRVIGEG |
| | | WNRAIGHHDPTAHAEMMALRQGGKVLENYRLLDTTLYVTFEP |
| | | CVMCAGAIVHSRIGRLVFGVRNGKTGAAGSLIDILGYPGMNH |
| | | QVIVSEGILAETCAGMLCYFFRQPREVKNALRKTRETPDR |
| T7.12 | 47 | MSDNNDEYWMRHALMLARRARDEGEVPVGAVLVLEGRVIGEG |
| | | WNRAIGHHDPTAHAEIMALRQGGKVIENYRLLDTTLYVTFEP |
| | | CVMCAGAMVHSRIGRLVFGVRNEKTGAAGSLLDVLGYPGMNH |
| | | QVQIEEGILAAECAAMLCYFFRHPRAVKNALRQAGKLL |
| T7.14 | 49 | MLPGVVCVTQEQDEYWMRRALTLAQRAREQGEVPVGAVLVQG |
| | | DRVIGEGWNRAIGQHDPTAHAEIMALRQGGKVLENYRLLNTT |
| | | LYVTFEPCIMCAGAMVHSRIGRLVYGVHNVKTGAAGSLIDIL |
| | | GYPGMNHQVALHQGVLEEECAAMLCYFFRMPRQQQNALRQAQ |
| | | RES |
| T7.15 | 50 | MNPQTDEYWMRYALELAKRAREQGEVPVGAVLVQGDKVIGEG |
| | | WNRAIGQHDPTAHAEIMALRQGGKVLENYRLLDTTLYVTFEP |
| | | CVMCAGAMVHGRITRLVYGVKNDKTGAAGSLLDVIGYPGMNH |
| | | QIQIDCGVLAEECAGMLCYFFRMPREQKNALRQAQRTG |
| T7.16 | 51 | MNQQRDEYWMRHALGLARLAREQGEVPVGAVLVQDERVIGEG |
| | | WNRAIGQHDPTAHAEMMALRQGGKVLENYRLLNTTLYVTFEP |
| | | CVMCAGAMVHSRITRLVYGVHNVKTGAAGSLLDVLGYPGMNH |
| | | QIELHSGVLAEECAAMLCYFFRMPREQKNALRQAQRQS |
| T7.17 | 52 | MNQQHDEYWMRHALGLARLAREQGEVPVGAVLVQADRVIGEG |
| | | WNRAIGQHDPTAHAEMMALRQGGKVLENYRLLNTTLYVTFEP |
| | | CVMCAGAMVHSRITRLVYGVHNVKTGAAGSLLDVLGYPGMNH |
| | | QVELHSGVLADECAAMLCYFFQMPREQKNALRQAQRQG |
| T7.24 | 59 | MEQIKLDPKTLHAKTNSSDELGCDEVSADVLAGEQPCLEQAS |
| | | LEQARVDEHWMRVAMTMAEKAEAAGEVPVGAVLVKDGQQIAA |
| | | GYNLSISEHDPCAHAEIQCLRAAGQTIENYRLLDTTLYVTFE |
| | | PCAMCAGAMVHSRIARVVFGVKNEKTGAAGTVLNLLQYPAFN |
| | | HQVEVTSGVLAQECADQLCRFFKRPREVKNALKQAQKAQQGT |
| | | LS |
| T8.1 | 71 | MPVQLAPGRVYNAPAFITGVTPLSDIQLSHEYWMRHALTLAK |
| | | RARDAREVPVGAVLVLNNRAIGEGWNRAIGHHDPTAHAEIMA |
| | | LRQGGLVLENYRLLDATLYVTFEPCVMCAGAMVHSRIARVVF |
| | | GVRNSKRGAAGSLMNVLNYPGMNHRVEVTEGVLGEECAALLC |
| | | EFYRMPRRVFNALKRASAQINEGQSAAAGPGER |
| T8.4 | 74 | MYNAPRFSTGVDALSETELNHEYWMRHALNLAQRAREEGEVP |
| | | VGAVLVYQDKVIGEGWNRAIGLHDPTAHAEIMALRQGGMVLQ |
| | | NYRLIDATLYVTFEPCVMCAGAMIHSRIGRVVFGVRNSKRGA |
| | | AGSLINVLNYPGMNHRVEVTEGVLAESCSSLLCDFYREPREQ |
| | | KNALKRASQDPRSSNA |
| T8.5 | 75 | MSDTEFTHEYWMQHALTLAQRARDEGEVPVGAVLVQDNRVIG |
| | | EGWNRAIGLHDPTAHAEIMALRQGGMVLQNYRLLNTTLYVTF |
| | | EPCIMCAGAMVHSRIGTLVFGVRNSKRGAVGSLMNVPAYPGM |
| | | NHHMQVIEGVLAPSCSALLCDFYRVPRLVKNAQKEASRKN |
| T8.6 | 76 | MSETEFSHEYWMRHALTLAQRARDEGEVPVGAVLVQDNRVIG |
| | | EGWNRAIGLHDPTAHAEIMALRQGGMVLQNYRLLNTTLYVTF |
| | | EPCIMCAGAMVHSRIGTLVFGVRNSKRGAVGSLMNIPAYPQM |
| | | NHHMQIIEGVLAPTCSALLCEFYRVPRLVKNAQKEALRKN |
| T8.7 | 77 | MSDNHDHEYWMQHALTLARRARDEGEVPVGAVLVRNGELIGE |
| | | GWNRAIGLHDPTAHAEIMALRQGGVVLQNYRLLDATLYVTFE |
| | | PCVMCAGAMVHSRIGRLVFGVRNSKRGAAGSLMNVLAYPGMN |
| | | HRVEVTEGVLAPACSTLLCDFYREPRLQQNALKEASRKPE |
| T8.8 | 78 | MTTPALTHEYWMNYALTLARRARDEGEVPVGAVLVYDNRVIG |
| | | EGWNRSIGKHDPTAHAEIMALRQGGMVQQNYRLLDTTLYVTF |
| | | EPCVMCAGAMIHSRIGTLVFGVRNSKRGAVGSQMNILNYPGM |
| | | NHQVQIIEGVLAPQCSALLCDFYRMPRKVKNALKIASRDPHP |
| | | AAPQR |
| T8.9 | 79 | MDNHDHEYWMQHALTLAQRAREEGEVPVGAVLVLNGQAIGEG |
| | | WNRAIGLHDPTAHAEMMALRQGGAVLQNYRLLNATLYVTFEP |
| | | CVMCAGAMVHSRIARLVFGVRNSKRGAAGSLLNVLAYPGMNH |
| | | RVAVTEGVLAPACSALLCDFYRQPRQQQNALKDANRKRN |
| T8.10 | 80 | MSDNNDEFWMRHALTLARRARQEGEVPVGAVLVLEGRVIGEG |
| | | WNRAIGHHDPTAHAEIMALRQGGKVIENYRLLDTTLYVTFEP |
| | | CVMCAGAMVHGRVGRLVFGVRNSKRGAAGSLINVLSYPGMNH |
| | | QVQVDEGVLAEECAAMLCDFYRQPRAVKNALRQQQQQGTSSA |
| | | E |
| T8.11 | 81 | MTDYNDELWMRHALTLARRARDEGEVPVGAVLVQDNRVIGEG |
| | | WNRAIGHHDPTAHAEMMALRQGGKVLENYRLLDTTLYVTFEP |
| | | CVMCAGAIVHSRIGRLVFGVRNGKRGAAGSLINILGYPGMNH |
| | | QVIVSEGILAETCAGMLCDFYRQPREVKNALRKTRETPNR |
| T8.12 | 82 | MSDNNDEYWMRHALMLARRARDEGEVPVGAVLVLEGRVIGEG |
| | | WNRAIGHHDPTAHAEIMALRQGGKVIENYRLLDTTLYVTFEP |
| | | CVMCAGAMVHSRIGRLVFGVRNEKRGAAGSLLNVLGYPGMNH |
| | | QVQIEEGILAAECAAMLCDFYRHPRAVKNALRQAGKLL |
| T8.14 | 84 | MLPGVVCVTQEQDEYWMRRALTLAQRAREQGEVPVGAVLVQG |
| | | DRVIGEGWNRAIGQHDPTAHAEIMALRQGGKVLENYRLLNTT |
| | | LYVTFEPCIMCAGAMVHSRIGRLVYGVHNVKRGAAGSLINIL |
| | | GYPGMNHQVALHQGVLEEECAAMLCDFYRMPRQQQNALRQAQ |
| | | RES |
| T8.15 | 85 | MNPQTDEYWMRYALELAKRAREQGEVPVGAVLVQGDKVIGEG |
| | | WNRAIGQHDPTAHAEIMALRQGGKVLENYRLLDTTLYVTFEP |
| | | CVMCAGAMVHGRITRLVYGVKNDKRGAAGSLLNVIGYPGMNH |
| | | QIQIDCGVLAEECAGMLCDFYRMPREQKNALRQAQRIG |
| T8.16 | 86 | MNQQRDEYWMRHALGLARLAREQGEVPVGAVLVQDERVIGEG |
| | | WNRAIGQHDPTAHAEMMALRQGGKVLENYRLLNTTLYVTFEP |
| | | CVMCAGAMVHSRITRLVYGVHNVKRGAAGSLLNVLGYPGMNH |
| | | QIELHSGVLAEECAAMLCDFYRMPREQKNALRQAQRQS |
| T8.17 | 87 | MNQQHDEYWMRHALGLARLAREQGEVPVGAVLVQADRVIGEG |
| | | WNRAIGQHDPTAHAEMMALRQGGKVLENYRLLNTTLYVTFEP |
| | | CVMCAGAMVHSRITRLVYGVHNVKRGAAGSLLNVLGYPGMNH |
| | | QVELHSGVLADECAAMLCDFYQMPREQKNALRQAQRQG |
| T8.24 | 94 | MEQIKLDPKTLHAKTNSSDELGCDEVSADVLAGEQPCLEQAS |
| | | LEQARVDEHWMRVAMTMAEKAEAAGEVPVGAVLVKDGQQIAA |
| | | GYNLSISEHDPCAHAEIQCLRAAGQTIENYRLLDTTLYVTFE |
| | | PCAMCAGAMVHSRIARVVFGVKNEKRGAAGTVLNLLQYPAFN |
| | | HQVEVTSGVLAQECADQLCRFYKRPREVKNALKQAQKAQQGI |
| | | LS |
| T8.101 | 116 | MTESETDHIRWMRHALSLAQRARDEGEVPVGAVLVYQGQVIG |
| | | EGWNRAIGHHDPTAHAEMMALRQGGIVLQNYRLLDTTLYVTF |
| | | EPCVMCAGAMVHSRIGQLIYGVSNVKRGAAGSLMNVLGYPGM |
| | | NHKVSVAGGVLAQECAGLLCDFYRMPRQVHNANKQAARQPSE |
| | | NQ |
| T8.102 | 117 | MTGSETDHIRWMRHALTLAQRARDEGEVPVGAVLVYQGQVIG |
| | | EGWNRAIGHHDPTAHAEMMALRQGGIVLQNYRLLDTTLYVTF |
| | | EPCVMCAGAMVHSRIGQLIYGVSNVKRGAAGSLMNVLGYPGM |
| | | NHKVSVAGGVLAQECAGLLCDFYRMPRQVHNANKQAARQQSE |
| | | NQ |
| T8.108 | 118 | MSDTLIDEKWMRHALTLARRAREEGEVPVGAVLVQGDTVIGE |
| | | GWNRAIGHHDPTAHAEIMALRQGGKVLENYRLLDTTLYVTFE |
| | | PCVMCAGAMVHGRVGRLVFGVRNEKRGAAGSLLNILGYAGMN |
| | | HQVRVDQGVLAAECAAMLCDFYRHPRAVKNALRDRLRAERLK |
| | | GE |
| T8.109 | 119 | MTDYNDEFWMRHALTLAQRARDEGEVPVGAVLVQGNRVIGEG |
| | | WNRAIGHHDPTAHAEIMALRQGGKVLENYRLIDTTLYVTFEP |
| | | CVMCAGAIVHSRIGRLVFGVRNEKRGAAGSLLNVLGYPGMNH |
| | | QVKMTEGILAQQCAGMLCDFYRMPREQKNALRKSVKQ |
| T8.110 | 120 | MTDHNDEYWMRHALRLARRARDEGEVPVGAVLVLDNQVIGEG |
| | | WNRAIGHHDPTAHAEMMALRQGGKMIENYRLLDTTLYVTFEP |
| | | CVMCAGAMIHSRIGRLVYGVRNSKRGAAGSLLNVLGYPGMNH |
| | | QVPAECGLLNDECAAMLCDFYRQPRAVKNMLRQQQNPGRV |
| T8.111 | 121 | MSDNNDEYWMRHALMLARRARDEGEVPVGAVLVLEGRAIGEG |
| | | WNRAIGHHDPTAHAEIMALRQGGKVIENYRLLNTTLYVTFEP |
| | | CVMCAGAMVHSRIGRLVFGVRNGKRGAAGSLLNVLGYPGMNH |
| | | QVQIEEGILATECAAMLCDFYRHPRAVKNAQRQAGKLL |
| T8.113 | 122 | MTQEQDEYWMRRALTLAQRAREQGEVPVGAVLVQGDRVIGEG |
| | | WNRAIGQHDPTAHAEIMALRQGGKVLENYRLLNTTLYVTFEP |
| | | CIMCAGAMVHSRIGRLVYGVHNSKRGAAGSLINILGYPGMNH |
| | | QVALHQGVLEEECAAMLCDFYRMPRQQQNALRQAQRES |
| T8.1-Q | 123 | MPVQLAPGRVYNAPAFITGVTPLSDIQLSHEYWMRHALTLAK |
| | | RARDAREVPVGAVLVLNNRAIGEGWNRAIGHHDPTAHAEIMA |
| | | LRQGGLVLENYRLLDATLYVTFEPCVMCAGAMVHSRIARVVF |
| | | GVRQSKRGAAGSLMNVLNYPGMNHRVEVTEGVLGEECAALLC |
| | | EFYRMPRRVFNALKRASAQINEGQSAAAGPGER |
| T8.4-Q | 124 | MYNAPRESTGVDALSETELNHEYWMRHALNLAQRAREEGEVP |
| | | VGAVLVYQDKVIGEGWNRAIGLHDPTAHAEIMALRQGGMVLQ |
| | | NYRLIDATLYVTFEPCVMCAGAMIHSRIGRVVFGVRQSKRGA |
| | | AGSLINVLNYPGMNHRVEVTEGVLAESCSSLLCDFYREPREQ |
| | | KNALKRASQDPRSSNA |
| T8.1-com1 | 125 | MPVQLAPGRVYNAPAFITGVTPLSDIQLSHEYWMRHALTLAK |
| | | RARDARAAPVGAVLVLNNRAIGEGWNRAIGHHDPTAHAEIIA |
| | | LRQGGLVLENYRLLDATLYVTFEPCVMCAGAMVNSRIARVVF |
| | | GVRNSKRGAAGSLMNVLNYPGMNHRVEVTEGVLGEECAALLC |
| | | EFYRMPRRVFNALKRASAQINEGQSAAAGPGER |
| T8.1-com2 | 126 | MPVQLAPGRVYNAPAFITGVTPLSDIQLSHEYWMRHALTLAK |
| | | RARDAGAGPVGAVLVLNNRAIGEGWNRAIGHHDPTAHAEIMA |
| | | LRQGGLVLENYRLFDATLYVTFEPCVMCAGAMVNSRIARVVF |
| | | GVRNSKRGAAGSLMNVLNYPGMNHRVEVTEGVLGEECAALLC |
| | | EFYRMPRRVENSLKRASAQINEGQSAAAGPGER |
| T8.4-com1 | 127 | MYNAPRFSTGVDALSETELNHEYWMRHALNLAQRAREEGAAP |
| | | VGAVLVYQDKVIGEGWNRAIGLHDPTAHAEIIALRQGGMVLQ |
| | | NYRLIDATLYVTFEPCVMCAGAMINSRIGRVVFGVRNSKRGA |
| | | AGSLINVLNYPGMNHRVEVTEGVLAESCSSLLCDFYREPREQ |
| | | KNALKRASQDPRSSNA |
| T8.4-com2 | 128 | MYNAPRFSTGVDALSETELNHEYWMRHALNLAQRAREEGAGP |
| | | VGAVLVYQDKVIGEGWNRAIGLHDPTAHAEIMALRQGGMVLQ |
| | | NYRLFDATLYVTFEPCVMCAGAMINSRIGRVVFGVRNSKRGA |
| | | AGSLINVLNYPGMNHRVEVTEGVLAESCSSLLCDFYREPREQ |
| | | KNSLKRASQDPRSSNA |
| T8.5-com1 | 129 | MSDTEFTHEYWMQHALTLAQRARDEGAAPVGAVLVQDNRVIG |
| | | EGWNRAIGLHDPTAHAEIIALRQGGMVLQNYRLLNTTLYVTF |
| | | EPCIMCAGAMVNSRIGTLVFGVRNSKRGAVGSLMNVPAYPGM |
| | | NHHMQVIEGVLAPSCSALLCDFYRVPRLVKNAQKEASRKN |
| T8.5-com2 | 130 | MSDTEFTHEYWMQHALTLAQRARDEGAGPVGAVLVQDNRVIG |
| | | EGWNRAIGLHDPTAHAEIMALRQGGMVLQNYRLENTTLYVTF |
| | | EPCIMCAGAMVNSRIGTLVFGVRNSKRGAVGSLMNVPAYPGM |
| | | NHHMQVIEGVLAPSCSALLCDFYRVPRLVKNSQKEASRKN |
| T8.8-com1 | 131 | MTTPALTHEYWMNYALTLARRARDEGAAPVGAVLVYDNRVIG |
| | | EGWNRSIGKHDPTAHAEIIALRQGGMVQQNYRLLDTTLYVTF |
| | | EPCVMCAGAMINSRIGTLVFGVRNSKRGAVGSQMNILNYPGM |
| | | NHQVQIIEGVLAPQCSALLCDFYRMPRKVKNALKIASRDPHP |
| | | AAPQR |
| T8.8-com2 | 132 | MTTPALTHEYWMNYALTLARRARDEGAGPVGAVLVYDNRVIG |
| | | EGWNRSIGKHDPTAHAEIMALRQGGMVQQNYRLFDTTLYVTF |
| | | EPCVMCAGAMINSRIGTLVFGVRNSKRGAVGSQMNILNYPGM |
| | | NHQVQIIEGVLAPQCSALLCDFYRMPRKVKNSLKIASRDPHP |
| | | AAPQR |
| T8.9-com1 | 133 | MDNHDHEYWMQHALTLAQRAREEGAAPVGAVLVLNGQAIGEG |
| | | WNRAIGLHDPTAHAEMIALRQGGAVLQNYRLLNATLYVTFEP |
| | | CVMCAGAMVNSRIARLVFGVRNSKRGAAGSLLNVLAYPGMNH |
| | | RVAVTEGVLAPACSALLCDFYRQPRQQQNALKDANRKRN |
| T8.9-com2 | 134 | MDNHDHEYWMQHALTLAQRAREEGAGPVGAVLVLNGQAIGEG |
| | | WNRAIGLHDPTAHAEMMALRQGGAVLQNYRLENATLYVTFEP |
| | | CVMCAGAMVNSRIARLVFGVRNSKRGAAGSLLNVLAYPGMNH |
| | | RVAVTEGVLAPACSALLCDFYRQPRQQQNSLKDANRKRN |
| T8.10-com1 | 135 | MSDNNDEFWMRHALTLARRARQEGAAPVGAVLVLEGRVIGEG |
| | | WNRAIGHHDPTAHAEIIALRQGGKVIENYRLLDTTLYVTFEP |
| | | CVMCAGAMVNGRVGRLVFGVRNSKRGAAGSLINVLSYPGMNH |
| | | QVQVDEGVLAEECAAMLCDFYRQPRAVKNALRQQQQQGTSSA |
| | | E |
| T8.10-com2 | 136 | MSDNNDEFWMRHALTLARRARQEGAGPVGAVLVLEGRVIGEG |
| | | WNRAIGHHDPTAHAEIMALRQGGKVIENYRLEDTTLYVTFEP |
| | | CVMCAGAMVNGRVGRLVFGVRNSKRGAAGSLINVLSYPGMNH |
| | | QVQVDEGVLAEECAAMLCDFYRQPRAVKNSLRQQQQQGTSSA |
| | | E |
| T8.12-com1 | 137 | MSDNNDEYWMRHALMLARRARDEGAAPVGAVLVLEGRVIGEG |
| | | WNRAIGHHDPTAHAEIIALRQGGKVIENYRLLDTTLYVTFEP |
| | | CVMCAGAMVNSRIGRLVFGVRNEKRGAAGSLLNVLGYPGMNH |
| | | QVQIEEGILAAECAAMLCDFYRHPRAVKNALRQAGKLL |
| T8.12-com2 | 138 | MSDNNDEYWMRHALMLARRARDEGAGPVGAVLVLEGRVIGEG |
| | | WNRAIGHHDPTAHAEIMALRQGGKVIENYRLFDTTLYVTFEP |
| | | CVMCAGAMVNSRIGRLVFGVRNEKRGAAGSLLNVLGYPGMNH |
| | | QVQIEEGILAAECAAMLCDFYRHPRAVKNSLRQAGKLL |
| T8.24-com1 | 139 | MEQIKLDPKTLHAKTNSSDELGCDEVSADVLAGEQPCLEQAS |
| | | LEQARVDEHWMRVAMTMAEKAEAAGAAPVGAVLVKDGQQIAA |
| | | GYNLSISEHDPCAHAEIQCLRAAGQTIENYRLLDTTLYVTFE |
| | | PCAMCAGAMVNSRIARVVFGVKNEKRGAAGTVLNLLQYPAFN |
| | | HQVEVTSGVLAQECADQLCRFYKRPREVKNALKQAQKAQQGI |
| | | LS |
| T8.24-com2 | 140 | MEQIKLDPKTLHAKTNSSDELGCDEVSADVLAGEQPCLEQAS |
| | | LEQARVDEHWMRVAMTMAEKAEAAGAGPVGAVLVKDGQQIAA |
| | | GYNLSISEHDPCAHAEIQCLRAAGQTIENYRLFDTTLYVTFE |
| | | PCAMCAGAMVNSRIARVVFGVKNEKRGAAGTVLNLLQYPAFN |
| | | HQVEVTSGVLAQECADQLCRFYKRPREVKNSLKQAQKAQQGI |
| | | LS |
| Traw.13 | 200 | MKQQDEYWMRHALSLARRAWEQGEVPVGAVLVQNDRVIGEGW |
| | | NRPIGQHDPTAHAEIMALRQGGKILENYRLLDTTLYVTLEPC |
| | | VMCAGAMVHSRIARLVYGAHDSKSGAAGSLLDVLGHPGMNHQ |
| | | VELHSGVLAEACAAMLSDFFRMRREQKKALRQAQRQG |
| Traw.23 | 201 | MSDQNSNRPTPNEDLANKQTQEKEAVQEPLTEIAMEDIATEE |
| | | DIMWMRHALTLADKAESIGEVPVGACVVLNGELIGEGENTPI |
| | | TDNDPSAHAELRAVKEAAAAVQNYRLIDATLYVTLEPCSMCA |
| | | GMLVHARVKRVVFGAKDAKTGAAGSVMNLLQHPALNHQLEVV |
| | | SGVLADECANKLSDFFRKRRKEIKAAKKAKRLLEGDASN |
| Traw.26 | 202 | MKFKQAESEQVQSKQAEIDVVSLSSVEKAPVSLDSPHFDQSK |
| | | VDKHWMRVAMAMAEKAEAEGEVPVGAVLVKDGQQIAAGYNLS |
| | | ISQHDPCAHAEILCLRAAGQTVENYRLLDATLYVTLEPCAMC |
| | | AGAMVHSRIARVVFGARDEKTGAAGTVLNLLQHPAFNHQVEV |
| | | TSGVLAQDCADQLSRFFKRRREEKKALKQAQKAQQERIS |
| Traw.28 | 203 | MEQTKLDPKTLHAKTHSSDELGCDEVSGGEQTCPKQAALEQA |
| | | QVDEHWMRVAMAMAEKAEAEGEVPVGAVLVKDGQQIATGYNL |
| | | SISQHDPCAHAEILCLRAAGQTVENYRLLDATLYVTLEPCAM |
| | | CAGAMVHSRIARVVFGARDEKTGAAGTVLNLLQHPVENHQVE |
| | | VTSGVLAQDCADQLSRFFKRRREEKKALKQAQKAQQERIS |
| Traw.30 | 204 | MEQIKLDPKTLHAKTHSSDELGCDDVSGGEQTCPKQADLEQA |
| | | QVDEHWMRVAMAMAEKAEAEGEVPVGAVLVKDGQQIAAGYNL |
| | | SISQHDPCAHAEILCLRAAGQTVENYRLLDATLYVTLEPCAM |
| | | CAGAMVHSRIARVVFGARDEKTGAAGTVLNLLQHPVENHQVE |
| | | VTSGVLAQDCADQLSRFFKRRREEKKALKQAQKAQQERIS |
| Traw.37 | 205 | MNPQTDEYWMRHALRLARLAWEQGEVPVGAVLVQGDTVIGEG |
| | | WNRPIGQHDPTAHAEIMALRQGGKVLENYRLLDTTLYVTLEP |
| | | CVMCAGAMVHSRITRLVYGAKDEKTGAAGSLLDVIGHPGMNH |
| | | QIQIDSGVLAEECAAMLSDFFRMRREQKKALRQAQRDAG |
| Traw.52 | 206 | MTDEDWMQYAIKLAAKAEEQGEVPVGAVLVKDGVMLSEGWNQ |
| | | MISLNDPSAHAEMQAIRAASALVGNYRLPDCTLYVTLEPCSM |
| | | CAGVMVHSRIKKVVFGASDLKTGAAGSVLNLLQHHCFNHQVE |
| | | IVPGVLAQQCAAQLSGFFQRRRAEHKALKRIQPQAD |
| Traw.54 | 207 | MTENRDLHWMQLAMEMAQKAEALGEVPVGAVLVKDDKLIACG |
| | | WNQPIAANDPCAHAEILCLRQAGSQLENYRLLDTTLYVTLEP |
| | | CAMCAGAMVHARVGRLVYGAADPKTGAAGSVLDLVRHPLENH |
| | | KLAVSAGVMEQECSEQLSAFFRRRRQEQKTLKQARKLNPPAA |
| | | EN |
| Traw.57 | 208 | MKQQDEYWMRHALSLARRAWEQGEVPVGAVLVQNDRVIGEGW |
| | | NRPIGQHDPTAHAEIMALRQGGKVLENYRLLETTLYVTLEPC |
| | | VMCAGAMVHSRITRLVYGAHDLKSGAAGSLLDVLGHPGMNHQ |
| | | VELASGVLAEECAAMLSDFFRMRREEKKALRQAQRQS |
| Traw.60 | 209 | MPACGIISVYNVAFFLLETRVTDRNDEYWMRHALQLARRAWN |
| | | EGEVPVGAVLVLDGQVIGEGWNRPIGHHDPTAHAEMMALRQG |
| | | GKVIENYRLMDTTLYVTLEPCVMCAGAMVHGRVGRLVYGARD |
| | | AKTGAAGSLLDVLGHPGMNHRVRVDCGVLADECAAMLSEFER |
| | | QRRAEKKALRQRQSTDGI |
| Traw.61 | 210 | MNHTLDGHDTPDEYWMRHALTLAQRAQEEGEVPVGAVLVLDN |
| | | QVIGEGWNRPIGHHDPTAHAEIMALRQGGNVLQNYRLINATL |
| | | YVTLEPCVMCAGAMVHSRIGRLVYGANDEKTGAAGSLVDILR |
| | | HPGMNHQITITSGVLAAECAHTLSDFFRIRRAQHKARRAQEK |
| | | ADGAA |
| Traw.62 | 211 | MNQSQDEYWMRHALRLARIAWEQGEVPVGAVLVQGDRVIGEG |
| | | WNRPIGQHDPTAHAEIMALRQGGKVLENYRLLDTTLYVTLEP |
| | | CVMCAGAMVHSRITRLVYGASDEKTGAAGSLIDVLGHPGMNH |
| | | QVALHAGVLAEECAAMLSDFFRMRREQKKALRQAQRGID |
| Traw.65 | 212 | MPAAFFPLESTVSEMSDEYWMRYALQLAHRAREQGEVPVGAV |
| | | LVQGSKVIGEGWNRPIGQHDPTAHAEMMALRQGGKVLENYRL |
| | | LDTTLYVTLEPCIMCAGAIVHSRIGRLVFGARDEKTGAAGSL |
| | | LDILGHPGMNHQVKVEHGLLAEECAAMLSDFFRMRREQKKAL |
| | | RQAQREAQQTPSS |
| Traw.74 | 213 | MNEQDEYWMRRAMALAARAEQEGEVPVGALVVYHGDCVGEGW |
| | | NRSIGHHDATAHAEIMALRQAGAHLGNYRLLECTLYVTLEPC |
| | | MMCAGAMVHSRIQRLVYGASDAKTGAVDSVLQLLATPGLNHR |
| | | VDWRAGVLADACSAQLSDFFRRRRAEKKAARKQAAGG |
| Traw.84 | 214 | MSDQNSNRPTPNEDLANKQTQEKEAVQEPLTEIAMEDIATKE |
| | | DIMWMRHALTLADKAESIGEVPVGACVVLNGELIGEGENTPI |
| | | TDNDPSAHAELRAVKEAAAAVQNYRLIDATLYVTLEPCSMCA |
| | | GMLVHARVKRVVFGAKDAKTGAAGSVMNLLQHPALNHQLEVV |
| | | SGVLADECANKLSDFFRKRRKEIKAAKKAKRLLEGDASS |
| Traw.103 | 215 | MDTLADDEYWMRHALILAQRAWDEGEVPVGAVLVQGGKVIGE |
| | | GWNRPIGQHDPTAHAEMMALRQGGRVLQNYRLLDTTLYITLE |
| | | PCIMCAGAMVHSRISRLVYGAADAKTGAAGSLVDILRHPGMN |
| | | HQVAITSGVLAEACSTLLSDFFRMRRQQQKAARAAGNA |
| Traw.105 | 216 | MNSVNDDEFWMQHALTLAQRAWDEGEVPVGAVLVLGNQVIGE |
| | | GWNRSIGQHDPTAHAEIMALRQGGMVVQNYRLLNSTLYITLE |
| | | PCVMCAGAMVHSRIGRLVYGAADAKTGAAGSLVDILRHPGMN |
| | | HQIEITSGVLAEACSTQLSDFFRMRRKEQKARRAAAKRESLS |
| Traw.112 | 217 | MTDNNDEFWMRHALQLAQRAWEEGEVPVGAVLVHQGRVIGEG |
| | | WNRPIGHHDPTAHAEMMAIRQGGKVIENYRLLDTTLYVTLEP |
| | | CVMCAGAMVHSRIGRLVFGARDAKTGAAGSLLDVLGHPGMNH |
| | | QVKFEHGILAEECAALLSDFFRQRRAEKKAQRQKNSGLV |
| Traw.114 | 218 | MTQEQDEYWMRRALTLAQRAWDEGEVPVGAVLVHGDRVIGEG |
| | | WNRPIGQHDPTAHAEIMALRQGGKVLENYRLLDTTLYVTLEP |
| | | CVMCAGAMVHSRIGRLVYGAHDVKTGAAGSLIDVLGHPGMNH |
| | | QVRLDHGVLEDACAALLSDFFRMRRQQQKALRQAQRES |
| Traw.107 | 219 | MTDQQDQYWMRYALQLAQRAWQAGEVPVGAVLVHDDRIIGEG |
| | | WNRPIGQHDPTAHAEIMALRQGGQALQNYRLLDATLYVTLEP |
| | | CTMCAGAMVHSRIGRLVFGANDEKTGAAGSLLNVLGHPGMNH |
| | | QVQVSSGILAQECAALLSDFFRMRREQKKARRSAPL |
| T8.26 | 220 | MKFKQAESEQVQSKQAEIDVVSLSSVEKAPVSLDSPHFDQSK |
| | | VDKHWMRVAMAMAEKAEAEGEVPVGAVLVKDGQQIAAGYNLS |
| | | ISQHDPCAHAEILCLRAAGQTVENYRLLDATLYVTFEPCAMC |
| | | AGAMVHSRIARVVFGVRNEKRGAAGTVLNLLQYPAFNHQVEV |
| | | TSGVLAQDCADQLCRFYKRPREVKNALKQAQKAQQERIS |
| T8.28 | 221 | MEQTKLDPKTLHAKTHSSDELGCDEVSGGEQTCPKQAALEQA |
| | | QVDEHWMRVAMAMAEKAEAEGEVPVGAVLVKDGQQIATGYNL |
| | | SISQHDPCAHAEILCLRAAGQTVENYRLLDATLYVTFEPCAM |
| | | CAGAMVHSRIARVVFGVRNEKRGAAGTVLNLLQYPVENHQVE |
| | | VTSGVLAQDCADQLCRFYKRPREVKNALKQAQKAQQERIS |
| T8.37 | 222 | MNPQTDEYWMRHALRLARLAREQGEVPVGAVLVQGDTVIGEG |
| | | WNRAIGQHDPTAHAEIMALRQGGKVLENYRLLDTTLYVTFEP |
| | | CVMCAGAMVHSRITRLVYGVKNEKRGAAGSLLNVIGYPGMNH |
| | | QIQIDSGVLAEECAAMLCDFYRMPREQKNALRQAQRDAG |
| T8.52 | 223 | MTDEDWMQYAIKLAAKAEEQGEVPVGAVLVKDGVMLSEGWNQ |
| | | MISLNDPSAHAEMQAIRAASALVGNYRLPDCTLYVTFEPCSM |
| | | CAGVMVHSRIKKVVFGVSNLKRGAAGSVLNLLQYHCENHQVE |
| | | IVPGVLAQQCAAQLCGFYQRPRAVHNALKRIQPQAN |
| T8.60 | 224 | MPACGIISVYNVAFFLLETRVTDRNDEYWMRHALQLARRARN |
| | | EGEVPVGAVLVLDGQVIGEGWNRAIGHHDPTAHAEMMALRQG |
| | | GKVIENYRLMDTTLYVTFEPCVMCAGAMVHGRVGRLVYGVRN |
| | | SKRGAAGSLLNVLGYPGMNHRVRVDCGVLADECAAMLCEFYR |
| | | QPRAVKNALRQRQSTDGI |
| T8.61 | 225 | MNHTLDGHDTPDEYWMRHALTLAQRAQEEGEVPVGAVLVLDN |
| | | QVIGEGWNRAIGHHDPTAHAEIMALRQGGNVLQNYRLINATL |
| | | YVTFEPCVMCAGAMVHSRIGRLVYGVNNEKRGAAGSLVNILR |
| | | YPGMNHQITITSGVLAAECAHTLCDFYRIPRAQHNARRAQEK |
| | | ADGAA |
| T8.62 | 226 | MNQSQDEYWMRHALRLARIAREQGEVPVGAVLVQGDRVIGEG |
| | | WNRAIGQHDPTAHAEIMALRQGGKVLENYRLLDTTLYVTFEP |
| | | CVMCAGAMVHSRITRLVYGVSNEKRGAAGSLINVLGYPGMNH |
| | | QVALHAGVLAEECAAMLCDFYRMPREQKNALRQAQRGIN |
| T8.65 | 227 | MPAAFFPLESTVSEMSDEYWMRYALQLAHRAREQGEVPVGAV |
| | | LVQGSKVIGEGWNRAIGQHDPTAHAEMMALRQGGKVLENYRL |
| | | LDTTLYVTFEPCIMCAGAIVHSRIGRLVFGVRNEKRGAAGSL |
| | | LNILGYPGMNHQVKVEHGLLAEECAAMLCDFYRMPREQKKAL |
| | | RQAQREAQQTPSS |
| T8.103 | 228 | MDTLADDEYWMRHALILAQRARDEGEVPVGAVLVQGGKVIGE |
| | | GWNRAIGQHDPTAHAEMMALRQGGRVLQNYRLLDTTLYITFE |
| | | PCIMCAGAMVHSRISRLVYGVANSKRGAAGSLVNILRYPGMN |
| | | HQVAITSGVLAEACSTLLCDFYRMPRQQQNAARAAGNA |
| T8.105 | 229 | MNSVNDDEFWMQHALTLAQRARDEGEVPVGAVLVLGNQVIGE |
| | | GWNRSIGQHDPTAHAEIMALRQGGMVVQNYRLLNSTLYITFE |
| | | PCVMCAGAMVHSRIGRLVYGVANSKRGAAGSLVNILRYPGMN |
| | | HQIEITSGVLAEACSTQLCDFYRMPRKVQNARRAAAKRESLS |
| T8.112 | 230 | MTDNNDEFWMRHALQLAQRAREEGEVPVGAVLVLQGRVIGEG |
| | | WNRAIGHHDPTAHAEMMAIRQGGKVIENYRLLDTTLYVTFEP |
| | | CVMCAGAMVHSRIGRLVFGVRNSKRGAAGSLLNVLGYPGMNH |
| | | QVKFEHGILAEECAALLCDFYRQPRAVKNAQRQKNSGLV |
| T8.114 | 231 | MTQEQDEYWMRRALTLAQRARDEGEVPVGAVLVLGDRVIGEG |
| | | WNRAIGQHDPTAHAEIMALRQGGKVLENYRLLDTTLYVTFEP |
| | | CVMCAGAMVHSRIGRLVYGVHNVKRGAAGSLINVLGYPGMNH |
| | | QVRLDHGVLEDACAALLCDFYRMPRQQQNALRQAQRES |
| T8.107 | 232 | MTDQQDQYWMRYALQLAQRARQAGEVPVGAVLVLDDRIIGEG |
| | | WNRAIGQHDPTAHAEIMALRQGGQALQNYRLLDATLYVTFEP |
| | | CTMCAGAMVHSRIGRLVFGVNNEKRGAAGSLLNVLGYPGMNH |
| | | QVQVSSGILAQECAALLCDFYRMPREQKNARRSAPL |
| T88.13 | 233 | MKQQDEYWMRHALSLARRAREQGEVPVGAVLVQNDRVIGEGW |
| | | NRAIGQHDPTAHAEIMALRQGGKILENYRLLDTTLYVTFEPC |
| | | VMCAGAMVHSRIARLVYGAHNSKSGAAGSLLDVLGHPGMNHQ |
| | | VELHSGVLAEACAAMLCRFFRMPRRQKNALRQAQRQG |
| T88.23 | 234 | MSDQNSNRPTPNFDLANKQTQEKEAVQEPLTEIAMEDIATEE |
| | | DIMWMRHALTLADKAESIGEVPVGACVVLNGELIGEGENTAI |
| | | TDNDPSAHAELRAVKEAAAAVQNYRLIDATLYVTFEPCSMCA |
| | | GMLVHARVKRVVVGAKNAKTGAAGSVMNLLQHPALNHQLEVV |
| | | SGVLADECANKLCRFFRKPRRVFNAAKKAKRLLEGDASN |
| T88.24 | 235 | MEQIKLDPKTLHAKTNSSDELGCDEVSADVLAGEQPCLEQAS |
| | | LEQARVDEHWMRVAMTMAEKAEAAGEVPVGAVLVKDGQQIAA |
| | | GYNLSISEHDPCAHAEIQCLRAAGQTIENYRLLDTTLYVTFE |
| | | PCAMCAGAMVHSRIARVVVGAKNEKTGAAGTVLNLLQHPAFN |
| | | HQVEVTSGVLAQECADQLCRFFKRPRRVKNALKQAQKAQQGT |
| | | LS |
| T88.30 | 236 | MEQIKLDPKTLHAKTHSSDELGCDDVSGGEQTCPKQADLEQA |
| | | QVDEHWMRVAMAMAEKAEAEGEVPVGAVLVKDGQQIAAGYNL |
| | | SISQHDPCAHAEILCLRAAGQTVENYRLLDATLYVTFEPCAM |
| | | CAGAMVHSRIARVVVGARNEKTGAAGTVLNLLQHPVENHQVE |
| | | VTSGVLAQDCADQLCRFFKRPRRVKNALKQAQKAQQERIS |
| T88.37 | 237 | MNPQTDEYWMRHALRLARLAREQGEVPVGAVLVQGDTVIGEG |
| | | WNRAIGQHDPTAHAEIMALRQGGKVLENYRLLDTTLYVTFEP |
| | | CVMCAGAMVHSRITRLVYGAKNEKTGAAGSLLDVIGHPGMNH |
| | | QIQIDSGVLAEECAAMLCRFFRMPRRQKNALRQAQRDAG |
| T88.52 | 238 | MTDEDWMQYAIKLAAKAEEQGEVPVGAVLVKDGVMLSEGWNQ |
| | | MISLNDPSAHAEMQAIRAASALVGNYRLPDCTLYVTFEPCSM |
| | | CAGVMVHSRIKKVVVGASNLKTGAAGSVLNLLQHHCENHQVE |
| | | IVPGVLAQQCAAQLCRFFQRPRRVHNALKRIQPQAD |
| T88.54 | 239 | MTENRDLHWMQLAMEMAQKAEALGEVPVGAVLVKDDKLIACG |
| | | WNQAIAANDPCAHAEILCLRQAGSQLENYRLLDTTLYVTFEP |
| | | CAMCAGAMVHARVGRLVYGAANPKTGAAGSVLDLVRHPLENH |
| | | KLAVSAGVMEQECSEQLCRFFRRPRRVQKTLNQARKLNPPAA |
| | | EN |
| T88.57 | 240 | MKQQDEYWMRHALSLARRAREQGEVPVGAVLVQNDRVIGEGW |
| | | NRAIGQHDPTAHAEIMALRQGGKVLENYRLLETTLYVTFEPC |
| | | VMCAGAMVHSRITRLVYGAHNLKSGAAGSLLDVLGHPGMNHQ |
| | | VELASGVLAEECAAMLCRFFRMPRRVKNALRQAQRQS |
| T88.74 | 241 | MNEQDEYWMRRAMALAARAEQEGEVPVGALVVYHGDCVGEGW |
| | | NRSIGHHDATAHAEIMALRQAGAHLGNYRLLECTLYVTFEPC |
| | | MMCAGAMVHSRIQRLVYGASNAKTGAVDSVLQLLAHPGLNHR |
| | | VDWRAGVLADACSAQLCRFFRRPRRVKNAARKQAAGG |
| T88.84 | 242 | MSDQNSNRPTPNEDLANKQTQEKEAVQEPLTEIAMEDIATKE |
| | | DIMWMRHALTLADKAESIGEVPVGACVVLNGELIGEGENTAI |
| | | TDNDPSAHAELRAVKEAAAAVQNYRLIDATLYVTFEPCSMCA |
| | | GMLVHARVKRVVVGAKNAKTGAAGSVMNLLQHPALNHQLEVV |
| | | SGVLADECANKLCRFFRKPRRVFNAAKKAKRLLEGDASS |
| T8.4-S15G- | 268 | MYNAPRFSTGVDALGETELNHEYWMRHALNLAQRAREEGE |
| S153R- | | VPVGAVLVYQDKVIGEGWNRAIGLHDPTAHAEIMALRQGGM |
| E164V- | | VLQNYRLIDATLYVTFEPCVMCAGAMIHSRIGRVVFGVRNSK |
| E167W | | RGAAGSLINVLNYPGMNHRVEVTEGVLAERCSSLLCDFYRVP |
| | | RWQKNALKRASQDPRSSNA |
| T8.4- | 269 | MYNAPRFSTGVDALSETELNHEYWMRHALNLAQRAREEGEV |
| C159R- | | PVGAVLVYQDKVIGEGWNRAIGLHDPTAHAEIMALRQGGMV |
| E164W- | | LQNYRLIDATLYVTFEPCVMCAGAMIHSRIGRVVFGVRNSKR |
| E167W | | GAAGSLINVLNYPGMNHRVEVTEGVLAESCSSLLRDFYRWPR |
| | | WQKNALKRASQDPRSSNA |
| T8.12- | 270 | MSDNNDEYWMRHALMLARRARDEGEVPVGAVLVLEGRVIGEG |
| V104M- | | WNRAIGHHDPTAHAEIMALRQGGKVIENYRLLDTTLYVTFEP |
| E107Y | | CVMCAGAMVHSRIGRLVFGMRNYKRGAAGSLLNVLGYPGMNH |
| | | QVQIEEGILAAECAAMLCDFYRHPRAVKNALRQAGKLL |
| T8.4-LbCpf1 | 271 | MKRTADGSEFESPKKKRKVMYNAPRFSTGVDALSETELNHE |
| | | YWMRHALNLAQRAREEGEVPVGAVLVYQDKVIGEGWNRAI |
| | | GLHDPTAHAEIMALRQGGMVLQNYRLIDATLYVTFEPCVMC |
| | | AGAMIHSRIGRVVFGVRNSKRGAAGSLINVLNYPGMNHRVE |
| | | VTEGVLAESCSSLLCDFYREPREQKNALKRASQDPRSSNAS |
| | | GGSSGGSSGSETPGTSESATPESSGGSSGGSSKLEKFTNC |
| | | YSLSKTLRFKAIPVGKTQENIDNKRLLVEDEKRAEDYKGVKKL |
| | | LDRYYLSFINDVLHSIKLKNLNNYISLFRKKTRTEKENKELENL |
| | | EINLRKEIAKAFKGNEGYKSLFKKDIIETILPEFLDDKDEIALVN |
| | | SFNGFTTAFTGFFDNRENMFSEEAKSTSIAFRCINENLTRYIS |
| | | NMDIFEKVDAIFDKHEVQEIKEKILNSDYDVEDFFEGEFFNFV |
| | | LTQEGIDVYNAIIGGFVTESGEKIKGLNEYINLYNQKTKQKLPK |
| | | FKPLYKQVLSDRESLSFYGEGYTSDEEVLEVFRNTLNKNSEI |
| | | FSSIKKLEKLFKNFDEYSSAGIFVKNGPAISTISKDIFGEWNVI |
| | | RDKWNAEYDDIHLKKKAVVTEKYEDDRRKSFKKIGSFSLEQL |
| | | QEYADADLSVVEKLKEIIIQKVDEIYKVYGSSEKLFDADFVLEK |
| | | SLKKNDAVVAIMKDLLDSVKSFENYIKAFFGEGKETNRDESF |
| | | YGDFVLAYDILLKVDHIYDAIRNYVTQKPYSKDKFKLYFQNPQ |
| | | FMGGWDKDKETDYRATILRYGSKYYLAIMDKKYAKCLQKIDK |
| | | DDVNGNYEKINYKLLPGPNKMLPKVFFSKKWMAYYNPSEDI |
| | | QKIYKNGTFKKGDMFNLNDCHKLIDFFKDSISRYPKWSNAYD |
| | | FNFSETEKYKDIAGFYREVEEQGYKVSFESASKKEVDKLVEE |
| | | GKLYMFQIYNKDFSDKSHGTPNLHTMYFKLLFDENNHGQIRL |
| | | SGGAELFMRRASLKKEELVVHPANSPIANKNPDNPKKTTTLS |
| | | YDVYKDKRFSEDQYELHIPIAINKCPKNIFKINTEVRVLLKHDD |
| | | NPYVIGIARGERNLLYIVVVDGKGNIVEQYSLNEIINNFNGIRIK |
| | | TDYHSLLDKKEKERFEARQNWTSIENIKELKAGYISQVVHKIC |
| | | ELVEKYDAVIALADLNSGFKNSRVKVEKQVYQKFEKMLIDKL |
| | | NYMVDKKSNPCATGGALKGYQITNKFESFKSMSTQNGFIFYI |
| | | PAWLTSKIDPSTGFVNLLKTKYTSIADSKKFISSFDRIMYVPEE |
| | | DLFEFALDYKNFSRTDADYIKKWKLYSYGNRIRIFRNPKKNN |
| | | VFDWEEVCLTSAYKELFNKYGINYQQGDIRALLCEQSDKAFY |
| | | SSFMALMSLMLQMRNSITGRTDVAFLISPVKNSDGIFYDSRN |
| | | YEAQENAILPKNADANGAYNIARKVLWAIGQFKKAEDEKLDK |
| | | VKIAISNKEWLEYAQTSVKHSGGSKRTADGSEFEPKKKRKV |
| T8.4-IscB- | 272 | MKRTADGSEFESPKKKRKVMYNAPRFSTGVDALSETELNHE |
| D61A | | YWMRHALNLAQRAREEGEVPVGAVLVYQDKVIGEGWNRAI |
| | | GLHDPTAHAEIMALRQGGMVLQNYRLIDATLYVTFEPCVMC |
| | | AGAMIHSRIGRVVFGVRNSKRGAAGSLINVLNYPGMNHRVE |
| | | VTEGVLAESCSSLLCDFYREPREQKNALKRASQDPRSSNAS |
| | | GGSSGGSSGSETPGTSESATPESSGGSSGGSMMAVVYVIS |
| | | KSGKPLMPTTRCGHVRILLKEGKARVVERKPFTIQLTYESAE |
| | | ETQPLVLGIAPGRTNIGMSVVTESGESVFNAQIRTRNKDVPK |
| | | LMKDRKQYRMAHRRLKRRCKRRRRAKAAGTAFEEGEKQRL |
| | | LPGCFKPITCKSIRNKEARFNNRKRPVGWLTPTANHLLVTHL |
| | | NVVKKVQKILPVAKVVLELNRFSFMAMNNPKVQRWQYQRG |
| | | PLYGKGSVEEAVSMQQDGHCLFCKHGIDHYHHVVPRRKNG |
| | | SETLENRVGLCEEHHRLVHTDKEWEANLASKKSGMNKKYH |
| | | ALSVLNQIIPYLADQLADMFPGNFCVTSGQDTYLFREEHGIPK |
| | | DHYLDAYCIACSALTDAKKVSSPKGRPYMVRQFRRHDRQAC |
| | | HKANLNRRYYMGGKLVATNRHKAMDQKTDSLEEYRAAHSA |
| | | ADVSKLTVKHPSAQYKDMSRIMPGSILVSGEGKLFTLRRSEG |
| | | RNKGQVNYFVSTEGIKYWARKCQYLRNNGGLQIYVSGGSK |
| | | RTADGSEFEPKKKRKV |
| T8.4_TnpB- | 273 | MKRTADGSEFESPKKKRKVMYNAPRFSTGVDALSETELNHE |
| D363A | | YWMRHALNLAQRAREEGEVPVGAVLVYQDKVIGEGWNRAI |
| | | GLHDPTAHAEIMALRQGGMVLQNYRLIDATLYVTFEPCVMC |
| | | AGAMIHSRIGRVVFGVRNSKRGAAGSLINVLNYPGMNHRVE |
| | | VTEGVLAESCSSLLCDFYREPREQKNALKRASQDPRSSNAS |
| | | GGSSGGSSGSETPGTSESATPESSGGSSGGSMGSPKKKRK |
| | | VEFMVNKSYKFRLYPTKEQEQLLAKTFGCVRFVYNKMLEERI |
| | | QIYEKFKDDKEALKKQTFPTPAKYKKEFPWLKEVDSLALANA |
| | | QLNLQKAFQNFFSGRAGFPKFKNRKAKQSYTTNVVNGNIQL |
| | | SDGYIKLPKLKWVKFKQHREIPAHHIIKACTIKKTKTGKYYVSI |
| | | LTEYEHQPVPKEIQTVVGLDFSMNGLFVDSEGKRANYPRFY |
| | | RQALEKLAKEQRILSRRKKGSNRWHKQRLKVAKLHEKIANQ |
| | | RKDFLHKKSYELAKQYDCVVIENLNMKGMSQVLNFGKSVHD |
| | | NGWGMFTTFLQYKLEEQGKKLIKIDKWFPSSKTCSCCDQVK |
| | | ESLSLSERTFRCECGFESDRAVNAAINIKHEGMKRLAIAGSK |
| | | RPAATKKAGQAKKKKTGYPYDVPDYAYPYDVPDYAYPYDVP |
| | | DYASGGSKRTADGSEFEPKKKRKV |

### Polynucleotide Sequence of ABE8e Expression Vector:

(SEQ ID NO: 106)
TCTAGAGGGCCCGTTTAAACCCGCTGATCAGCCTCGACTGTGCCTTCTAGTTGCCAGCC
ATCTGTTGTTTGCCCCTCCCCCGTGCCTTCCTTGACCCTGGAAGGTGCCACTCCCACTG
TCCTTTCCTAATAAAATGAGGAAATTGCATCGCATTGTCTGAGTAGGTGTCATTCTATT
CTGGGGGGTGGGGTGGGGCAGGACAGCAAGGGGGAGGATTGGGAAGACAATAGCAGGCA
TGCTGGGGATGCGGTGGGCTCTATGGTTTGTTACTTTATAGAAGAAATTTTGAGTTTTT
GTTTTTTTTTAATAAATAAATAAACATAAATAAATTGTTTGTTGAATTTATTATTAGTA
TGTAAGTGTAAATATAATAAAACTTAATATCTATTCAAATTAATAAATAAACCTCGATA
TACAGACCGATAAAACACATGCGTCAATTTTACGCATGATTATCTTTAACGTACGTCAC
AATATGATTATCTTTCTAGGGTTAACTTCTACTGGGCGGTTTTATGGACAGCAAGCGAA
CCGGAATTGCCAGCTGGGGCGCCCTCTGGTAAGGTTGGGAAGCCCTGCAAAGTAAACTG
GATGGCTTTCTCGCCGCCAAGGATCTGATGGCGCAGGGGATCAAGCTCTGATCAAGAGA
CAGGATGAGGATCGTTTCGCATGATTGAACAAGATGGATTGCACGCAGGTTCTCCGGCC
GCTTGGGTGGAGAGGCTATTCGGCTATGACTGGGCACAACAGACAATCGGCTGCTCTGA
TGCCGCCGTGTTCCGGCTGTCAGCGCAGGGGCGCCCGGTTCTTTTTGTCAAGACCGACC
TGTCCGGTGCCCTGAATGAACTGCAAGACGAGGCAGCGCGGCTATCGTGGCTGGCCACG
ACGGGCGTTCCTTGCGCAGCTGTGCTCGACGTTGTCACTGAAGCGGGAAGGGACTGGCT
GCTATTGGGCGAAGTGCCGGGGCAGGATCTCCTGTCATCTCACCTTGCTCCTGCCGAGA
AAGTATCCATCATGGCTGATGCAATGCGGCGGCTGCATACGCTTGATCCGGCTACCTGC
CCATTCGACCACCAAGCGAAACATCGCATCGAGCGAGCACGTACTCGGATGGAAGCCGG
TCTTGTCGATCAGGATGATCTGGACGAAGAGCATCAGGGGCTCGCGCCAGCCGAACTGT
TCGCCAGGCTCAAGGCGAGCATGCCCGACGGCGAGGATCTCGTCGTGACCCATGGCGAT
GCCTGCTTGCCGAATATCATGGTGGAAAATGGCCGCTTTTCTGGATTCATCGACTGTGG
CCGGCTGGGTGTGGCGGACCGCTATCAGGACATAGCGTTGGCTACCCGTGATATTGCTG
AAGAGCTTGGCGGCGAATGGGCTGACCGCTTCCTCGTGCTTTACGGTATCGCCGCTCCC
GATTCGCAGCGCATCGCCTTCTATCGCCTTCTTGACGAGTTCTTCTGAATTATTAACGC
TTACAATTTCCTGATGCGGTATTTTCTCCTTACGCATCTGTGCGGTATTTCACACCGCA
TACAGGTGGCACTTTTCGGGGAAATGTGCGCGGAACCCCTATTTGTTTATTTTTCTAAA
TACATTCAAATATGTATCCGCTCATGAGACAATAACCCTGATAAATGCTTCAATAATAG
CACGTGCTAAAACTTCATTTTTAATTTAAAAGGATCTAGGTGAAGATCCTTTTTGATAA
TCTCATGACCAAAATCCCTTAACGTGAGTTTTCGTTCCACTGAGCGTCAGACCCCGTAG
AAAAGATCAAAGGATCTTCTTGAGATCCTTTTTTTCTGCGCGTAATCTGCTGCTTGCAA
ACAAAAAAACCACCGCTACCAGCGGTGGTTTGTTTGCCGGATCAAGAGCTACCAACTCT
TTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTG
CTAATCCTGTTACCAGTGGCTGCTGCCAGTGGCGATAAGTCGTGTCTTACCGGGTTGGA
CTCAAGACGATAGTTACCGGATAAGGCGCAGCGGTCGGGCTGAACGGGGGGTTCGTGCA
CACAGCCCAGCTTGGAGCGAACGACCTACACCGAACTGAGATACCTACAGCGTGAGCTA
TGAGAAAGCGCCACGCTTCCCGAAGGGAGAAAGGCGGACAGGTATCCGGTAAGCGGCAG
GGTCGGAACAGGAGAGCGCACGAGGGAGCTTCCAGGGGGAAACGCCTGGTATCTTTATA
GTCCTGTCGGGTTTCGCCACCTCTGACTTGAGCGTCGATTTTTGTGATGCTCGTCAGGG
GGGCGGAGCCTATGGAAAAACGCCAGCAACGCGGCCTTTTTACGGTTCCTGGGCTTTTG
CTGGCCTTTTGCTCACATGTTCTTGCTGCTTCGCGATGTACGGGCCAGATATATTAACC
CTAGAAAGATAGTCTGCGTAAAATTGACGCATGCATTCTTGAAATATTGCTCTCTCTTT
CTAAATAGCGCGAATCCGTCGCTGTGCATTTAGGACATCTCAGTCGCCGCTTGGAGCTC
CCGTGAGGCGTGCTTGTCAATGCGGTAAGTGTCACTGATTTTGAACTATAACGACCGCG
TGAGTCAAAATGACGCATGATTATCTTTTACGTGACTTTTAAGATTTAACTCATACGAT
AATTATATTGTTATTTCATGTTCTACTTACGTGATAACTTATTATATATATATTTTCTT
GTTATATGCAGATATACGCGTTGACATTGATTATTGACTAGTTATTAATAGTAATCAAT
TACGGGGTCATTAGTTCATAGCCCATATATGGAGTTCCGCGTTACATAACTTACGGTAA
ATGGCCCGCCTGGCTGACCGCCCAACGACCCCCGCCCATTGACGTCAATAATGACGTAT
GTTCCCATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCTATTG
ACGTCAATGACGGTAAATGGCCCGCCTGGCATTATGCCCAGTACATGACCTTATGGGAC
TTTCCTACTTGGCAGTACATCTACGTATTAGTCATCGCTATTACCATGGTGATGCGGTT
TTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAA
TGTCGTAACAACTCCGCCCCATTGACGCAAATGGGCGGTAGGCGTGTACGGTGGGAGGT
CTATATAAGCAGAGCTGGTTTAGTGAACCGTCAGATCCGCTAGAGATCCGCGGCCGCTA
ATACGACTCACTATAGGGAGAGCCGCCACCATGAAACGGACAGCCGACGGAAGCGAGTT
CGAGTCACCAAAGAAGAAGCGGAAAGTCTCTGAGGTGGAGTTTTCCCACGAGTACTGGA
TGAGACATGCCCTGACCCTGGCCAAGAGGGCACGGGATGAGAGGGAGGTGCCTGTGGGA
GCCGTGCTGGTGCTGAACAATAGAGTGATCGGCGAGGGCTGGAACAGAGCCATCGGCCT
GCACGACCCAACAGCCCATGCCGAAATTATGGCCCTGAGACAGGGCGGCCTGGTCATGC
AGAACTACAGACTGATTGACGCCACCCTGTACGTGACATTCGAGCCTTGCGTGATGTGC
GCCGGCGCCATGATCCACTCTAGGATCGGCCGCGTGGTGTTTGGCGTGAGGAACTCAAA
AAGAGGCGCCGCAGGCTCCCTGATGAACGTGCTGAACTACCCCGGCATGAATCACCGCG
TCGAAATTACCGAGGGAATCCTGGCAGATGAATGTGCCGCCCTGCTGTGCGATTTCTAT
CGGATGCCTAGACAGGTGTTCAATGCTCAGAAGAAGGCCCAGAGCTCCATCAACTCCGG
AGGATCTAGCGGAGGCTCCTCTGGCTCTGAGACACCTGGCACAAGCGAGAGCGCAACAC
CTGAAAGCAGCGGGGGCAGCAGCGGGGGGTCAGACAAGAAGTACAGCATCGGCCTGGCC
ATCGGCACCAACTCTGTGGGCTGGGCCGTGATCACCGACGAGTACAAGGTGCCCAGCAA
GAAATTCAAGGTGCTGGGCAACACCGACCGGCACAGCATCAAGAAGAACCTGATCGGAG
CCCTGCTGTTCGACAGCGGCGAAACAGCCGAGGCCACCCGGCTGAAGAGAACCGCCAGA
AGAAGATACACCAGACGGAAGAACCGGATCTGCTATCTGCAAGAGATCTTCAGCAACGA
GATGGCCAAGGTGGACGACAGCTTCTTCCACAGACTGGAAGAGTCCTTCCTGGTGGAAG
AGGATAAGAAGCACGAGCGGCACCCCATCTTCGGCAACATCGTGGACGAGGTGGCCTAC
CACGAGAAGTACCCCACCATCTACCACCTGAGAAAGAAACTGGTGGACAGCACCGACAA
GGCCGACCTGCGGCTGATCTATCTGGCCCTGGCCCACATGATCAAGTTCCGGGGCCACT
TCCTGATCGAGGGCGACCTGAACCCCGACAACAGCGACGTGGACAAGCTGTTCATCCAG
CTGGTGCAGACCTACAACCAGCTGTTCGAGGAAAACCCCATCAACGCCAGCGGCGTGGA
CGCCAAGGCCATCCTGTCTGCCAGACTGAGCAAGAGCAGACGGCTGGAAAATCTGATCG
CCCAGCTGCCCGGCGAGAAGAAGAATGGCCTGTTCGGAAACCTGATTGCCCTGAGCCTG
GGCCTGACCCCCAACTTCAAGAGCAACTTCGACCTGGCCGAGGATGCCAAACTGCAGCT
GAGCAAGGACACCTACGACGACGACCTGGACAACCTGCTGGCCCAGATCGGCGACCAGT
ACGCCGACCTGTTTCTGGCCGCCAAGAACCTGTCCGACGCCATCCTGCTGAGCGACATC
CTGAGAGTGAACACCGAGATCACCAAGGCCCCCCTGAGCGCCTCTATGATCAAGAGATA
CGACGAGCACCACCAGGACCTGACCCTGCTGAAAGCTCTCGTGCGGCAGCAGCTGCCTG
AGAAGTACAAAGAGATTTTCTTCGACCAGAGCAAGAACGGCTACGCCGGCTACATTGAC
GGCGGAGCCAGCCAGGAAGAGTTCTACAAGTTCATCAAGCCCATCCTGGAAAAGATGGA
CGGCACCGAGGAACTGCTCGTGAAGCTGAACAGAGAGGACCTGCTGCGGAAGCAGCGGA
CCTTCGACAACGGCAGCATCCCCCACCAGATCCACCTGGGAGAGCTGCACGCCATTCTG
CGGCGGCAGGAAGATTTTTACCCATTCCTGAAGGACAACCGGGAAAAGATCGAGAAGAT
CCTGACCTTCCGCATCCCCTACTACGTGGGCCCTCTGGCCAGGGGAAACAGCAGATTCG
CCTGGATGACCAGAAAGAGCGAGGAAACCATCACCCCCTGGAACTTCGAGGAAGTGGTG
GACAAGGGCGCTTCCGCCCAGAGCTTCATCGAGCGGATGACCAACTTCGATAAGAACCT
GCCCAACGAGAAGGTGCTGCCCAAGCACAGCCTGCTGTACGAGTACTTCACCGTGTATA
ACGAGCTGACCAAAGTGAAATACGTGACCGAGGGAATGAGAAAGCCCGCCTTCCTGAGC
GGCGAGCAGAAAAAGGCCATCGTGGACCTGCTGTTCAAGACCAACCGGAAAGTGACCGT
GAAGCAGCTGAAAGAGGACTACTTCAAGAAAATCGAGTGCTTCGACTCCGTGGAAATCT
CCGGCGTGGAAGATCGGTTCAACGCCTCCCTGGGCACATACCACGATCTGCTGAAAATT
ATCAAGGACAAGGACTTCCTGGACAATGAGGAAAACGAGGACATTCTGGAAGATATCGT
GCTGACCCTGACACTGTTTGAGGACAGAGAGATGATCGAGGAACGGCTGAAAACCTATG
CCCACCTGTTCGACGACAAAGTGATGAAGCAGCTGAAGCGGCGGAGATACACCGGCTGG
GGCAGGCTGAGCCGGAAGCTGATCAACGGCATCCGGGACAAGCAGTCCGGCAAGACAAT
CCTGGATTTCCTGAAGTCCGACGGCTTCGCCAACAGAAACTTCATGCAGCTGATCCACG
ACGACAGCCTGACCTTTAAAGAGGACATCCAGAAAGCCCAGGTGTCCGGCCAGGGCGAT
AGCCTGCACGAGCACATTGCCAATCTGGCCGGCAGCCCCGCCATTAAGAAGGGCATCCT
GCAGACAGTGAAGGTGGTGGACGAGCTCGTGAAAGTGATGGGCCGGCACAAGCCCGAGA
ACATCGTGATCGAAATGGCCAGAGAGAACCAGACCACCCAGAAGGGACAGAAGAACAGC
CGCGAGAGAATGAAGCGGATCGAAGAGGGCATCAAAGAGCTGGGCAGCCAGATCCTGAA
AGAACACCCCGTGGAAAACACCCAGCTGCAGAACGAGAAGCTGTACCTGTACTACCTGC
AGAATGGGCGGGATATGTACGTGGACCAGGAACTGGACATCAACCGGCTGTCCGACTAC
GATGTGGACCATATCGTGCCTCAGAGCTTTCTGAAGGACGACTCCATCGACAACAAGGT
GCTGACCAGAAGCGACAAGAACCGGGGCAAGAGCGACAACGTGCCCTCCGAAGAGGTCG
TGAAGAAGATGAAGAACTACTGGCGGCAGCTGCTGAACGCCAAGCTGATTACCCAGAGA
AAGTTCGACAATCTGACCAAGGCCGAGAGAGGCGGCCTGAGCGAACTGGATAAGGCCGG
CTTCATCAAGAGACAGCTGGTGGAAACCCGGCAGATCACAAAGCACGTGGCACAGATCC
TGGACTCCCGGATGAACACTAAGTACGACGAGAATGACAAGCTGATCCGGGAAGTGAAA
GTGATCACCCTGAAGTCCAAGCTGGTGTCCGATTTCCGGAAGGATTTCCAGTTTTACAA
AGTGCGCGAGATCAACAACTACCACCACGCCCACGACGCCTACCTGAACGCCGTCGTGG
GAACCGCCCTGATCAAAAAGTACCCTAAGCTGGAAAGCGAGTTCGTGTACGGCGACTAC
AAGGTGTACGACGTGCGGAAGATGATCGCCAAGAGCGAGCAGGAAATCGGCAAGGCTAC
CGCCAAGTACTTCTTCTACAGCAACATCATGAACTTTTTCAAGACCGAGATTACCCTGG
CCAACGGCGAGATCCGGAAGCGGCCTCTGATCGAGACAAACGGCGAAACCGGGGAGATC
GTGTGGGATAAGGGCCGGGATTTTGCCACCGTGCGGAAAGTGCTGAGCATGCCCCAAGT
GAATATCGTGAAAAAGACCGAGGTGCAGACAGGCGGCTTCAGCAAAGAGTCTATCCTGC
CCAAGAGGAACAGCGATAAGCTGATCGCCAGAAAGAAGGACTGGGACCCTAAGAAGTAC
GGCGGCTTCGACAGCCCCACCGTGGCCTATTCTGTGCTGGTGGTGGCCAAAGTGGAAAA
GGGCAAGTCCAAGAAACTGAAGAGTGTGAAAGAGCTGCTGGGGATCACCATCATGGAAA
GAAGCAGCTTCGAGAAGAATCCCATCGACTTTCTGGAAGCCAAGGGCTACAAAGAAGTG
AAAAAGGACCTGATCATCAAGCTGCCTAAGTACTCCCTGTTCGAGCTGGAAAACGGCCG
GAAGAGAATGCTGGCCTCTGCCGGCGAACTGCAGAAGGGAAACGAACTGGCCCTGCCCT
CCAAATATGTGAACTTCCTGTACCTGGCCAGCCACTATGAGAAGCTGAAGGGCTCCCCC
GAGGATAATGAGCAGAAACAGCTGTTTGTGGAACAGCACAAGCACTACCTGGACGAGAT
CATCGAGCAGATCAGCGAGTTCTCCAAGAGAGTGATCCTGGCCGACGCTAATCTGGACA
AAGTGCTGTCCGCCTACAACAAGCACCGGGATAAGCCCATCAGAGAGCAGGCCGAGAAT
ATCATCCACCTGTTTACCCTGACCAATCTGGGAGCCCCTGCCGCCTTCAAGTACTTTGA
CACCACCATCGACCGGAAGAGGTACACCAGCACCAAAGAGGTGCTGGACGCCACCCTGA
TCCACCAGAGCATCACCGGCCTGTACGAGACACGGATCGACCTGTCTCAGCTGGGAGGT
GACTCTGGCGGCTCAAAAAGAACCGCCGACGGCAGCGAATTCGAGCCCAAGAAGAAGAG
GAAAGTCGGAAGCGGAGCTACTAACTTCAGCCTGCTGAAGCAGGCTGGAGACGTGGAGG
AGAACCCTGGACCTATGAGCGAGCTGATCAAGGAGAACATGCACATGAAGCTGTACATG
GAGGGCACCGTGAACAACCACCACTTCAAGTGCACATCCGAGGGCGAAGGCAAGCCCTA
CGAGGGCACCCAGACCATGAAGATCAAGGTGGTCGAGGGCGGCCCTCTCCCCTTCGCCT
TCGACATCCTGGCTACCAGCTTCATGTACGGCAGCAAAGCCTTCATCAACCACACCCAG
GGCATCCCCGACTTCTTTAAGCAGTCCTTCCCTGAGGGCTTCACATGGGAGAGAATCAC
CACATACGAAGACGGGGGCGTGCTGACCGCTACCCAGGACACCAGCTTCCAGAACGGCT
GCATCATCTACAACGTCAAGATCAACGGGGTGAACTTCCCATCCAACGGCCCTGTGATG
CAGAAGAAAACACGCGGCTGGGAGGCCAACACCGAGATGCTGTACCCCGCTGACGGCGG
CCTGAGAGGCCACAGCCAGATGGCCCTGAAGCTCGTGGGCGGGGGCTACCTGCACTGCT
CCTTCAAGACCACATACAGATCCAAGAAACCCGCTAAGAACCTCAAGATGCCCGGCTTC
CACTTCGTGGACCACAGACTGGAAAGAATCAAGGAGGCCGACAAAGAGACCTACGTCGA
GCAGCACGAGATGGCTGTGGCCAAGTACTGCGACCTCCCTAGCAAACTGGGGCACAGAT
CTGGTGGTTCTCCCAAGAAGAAGAGGAAAGTCTAA

### Polynucleotide Sequence of BE4max Expression Vector:

(SEQ ID NO: 142)
TCTAGAGGGCCCGTTTAAACCCGCTGATCAGCCTCGACTGTGCCTTCTAGTTGCCAGCC
ATCTGTTGTTTGCCCCTCCCCCGTGCCTTCCTTGACCCTGGAAGGTGCCACTCCCACTG
TCCTTTCCTAATAAAATGAGGAAATTGCATCGCATTGTCTGAGTAGGTGTCATTCTATT
CTGGGGGGTGGGGTGGGGCAGGACAGCAAGGGGGAGGATTGGGAAGACAATAGCAGGCA
TGCTGGGGATGCGGTGGGCTCTATGGTTTGTTACTTTATAGAAGAAATTTTGAGTTTTT
GTTTTTTTTTAATAAATAAATAAACATAAATAAATTGTTTGTTGAATTTATTATTAGTA
TGTAAGTGTAAATATAATAAAACTTAATATCTATTCAAATTAATAAATAAACCTCGATA
TACAGACCGATAAAACACATGCGTCAATTTTACGCATGATTATCTTTAACGTACGTCAC
AATATGATTATCTTTCTAGGGTTAACTTCTACTGGGCGGTTTTATGGACAGCAAGCGAA
CCGGAATTGCCAGCTGGGGCGCCCTCTGGTAAGGTTGGGAAGCCCTGCAAAGTAAACTG
GATGGCTTTCTCGCCGCCAAGGATCTGATGGCGCAGGGGATCAAGCTCTGATCAAGAGA
CAGGATGAGGATCGTTTCGCATGATTGAACAAGATGGATTGCACGCAGGTTCTCCGGCC
GCTTGGGTGGAGAGGCTATTCGGCTATGACTGGGCACAACAGACAATCGGCTGCTCTGA
TGCCGCCGTGTTCCGGCTGTCAGCGCAGGGGCGCCCGGTTCTTTTTGTCAAGACCGACC
TGTCCGGTGCCCTGAATGAACTGCAAGACGAGGCAGCGCGGCTATCGTGGCTGGCCACG
ACGGGCGTTCCTTGCGCAGCTGTGCTCGACGTTGTCACTGAAGCGGGAAGGGACTGGCT
GCTATTGGGCGAAGTGCCGGGGCAGGATCTCCTGTCATCTCACCTTGCTCCTGCCGAGA
AAGTATCCATCATGGCTGATGCAATGCGGCGGCTGCATACGCTTGATCCGGCTACCTGC
CCATTCGACCACCAAGCGAAACATCGCATCGAGCGAGCACGTACTCGGATGGAAGCCGG
TCTTGTCGATCAGGATGATCTGGACGAAGAGCATCAGGGGCTCGCGCCAGCCGAACTGT
TCGCCAGGCTCAAGGCGAGCATGCCCGACGGCGAGGATCTCGTCGTGACCCATGGCGAT
GCCTGCTTGCCGAATATCATGGTGGAAAATGGCCGCTTTTCTGGATTCATCGACTGTGG
CCGGCTGGGTGTGGCGGACCGCTATCAGGACATAGCGTTGGCTACCCGTGATATTGCTG
AAGAGCTTGGCGGCGAATGGGCTGACCGCTTCCTCGTGCTTTACGGTATCGCCGCTCCC
GATTCGCAGCGCATCGCCTTCTATCGCCTTCTTGACGAGTTCTTCTGAATTATTAACGC
TTACAATTTCCTGATGCGGTATTTTCTCCTTACGCATCTGTGCGGTATTTCACACCGCA
TACAGGTGGCACTTTTCGGGGAAATGTGCGCGGAACCCCTATTTGTTTATTTTTCTAAA
TACATTCAAATATGTATCCGCTCATGAGACAATAACCCTGATAAATGCTTCAATAATAG
CACGTGCTAAAACTTCATTTTTAATTTAAAAGGATCTAGGTGAAGATCCTTTTTGATAA
TCTCATGACCAAAATCCCTTAACGTGAGTTTTCGTTCCACTGAGCGTCAGACCCCGTAG
AAAAGATCAAAGGATCTTCTTGAGATCCTTTTTTTCTGCGCGTAATCTGCTGCTTGCAA
ACAAAAAAACCACCGCTACCAGCGGTGGTTTGTTTGCCGGATCAAGAGCTACCAACTCT
TTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTG
CTAATCCTGTTACCAGTGGCTGCTGCCAGTGGCGATAAGTCGTGTCTTACCGGGTTGGA
CTCAAGACGATAGTTACCGGATAAGGCGCAGCGGTCGGGCTGAACGGGGGGTTCGTGCA
CACAGCCCAGCTTGGAGCGAACGACCTACACCGAACTGAGATACCTACAGCGTGAGCTA
TGAGAAAGCGCCACGCTTCCCGAAGGGAGAAAGGCGGACAGGTATCCGGTAAGCGGCAG
GGTCGGAACAGGAGAGCGCACGAGGGAGCTTCCAGGGGGAAACGCCTGGTATCTTTATA
GTCCTGTCGGGTTTCGCCACCTCTGACTTGAGCGTCGATTTTTGTGATGCTCGTCAGGG
GGGCGGAGCCTATGGAAAAACGCCAGCAACGCGGCCTTTTTACGGTTCCTGGGCTTTTG
CTGGCCTTTTGCTCACATGTTCTTGCTGCTTCGCGATGTACGGGCCAGATATATTAACC
CTAGAAAGATAGTCTGCGTAAAATTGACGCATGCATTCTTGAAATATTGCTCTCTCTTT
CTAAATAGCGCGAATCCGTCGCTGTGCATTTAGGACATCTCAGTCGCCGCTTGGAGCTC
CCGTGAGGCGTGCTTGTCAATGCGGTAAGTGTCACTGATTTTGAACTATAACGACCGCG
TGAGTCAAAATGACGCATGATTATCTTTTACGTGACTTTTAAGATTTAACTCATACGAT
AATTATATTGTTATTTCATGTTCTACTTACGTGATAACTTATTATATATATATTTTCTT
GTTATATGCAGATATACGCGTTGACATTGATTATTGACTAGTTATTAATAGTAATCAAT
TACGGGGTCATTAGTTCATAGCCCATATATGGAGTTCCGCGTTACATAACTTACGGTAA
ATGGCCCGCCTGGCTGACCGCCCAACGACCCCCGCCCATTGACGTCAATAATGACGTAT
GTTCCCATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCTATTG
ACGTCAATGACGGTAAATGGCCCGCCTGGCATTATGCCCAGTACATGACCTTATGGGAC
TTTCCTACTTGGCAGTACATCTACGTATTAGTCATCGCTATTACCATGGTGATGCGGTT
TTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAA
TGTCGTAACAACTCCGCCCCATTGACGCAAATGGGCGGTAGGCGTGTACGGTGGGAGGT
CTATATAAGCAGAGCTGGTTTAGTGAACCGTCAGATCCGCTAGAGATCCGCGGCCGCTA
ATACGACTCACTATAGGGAGAGCCGCCACCATGAAACGGACAGCCGACGGAAGCGAGTT
CGAGTCACCAAAGAAGAAGCGGAAAGTCTCCTCAGAGACTGGGCCTGTCGCCGTCGATC
CAACCCTGCGCCGCCGGATTGAACCTCACGAGTTTGAAGTGTTCTTTGACCCCCGGGAG
CTGAGAAAGGAGACATGCCTGCTGTACGAGATCAACTGGGGAGGCAGGCACTCCATCTG
GAGGCACACCTCTCAGAACACAAATAAGCACGTGGAGGTGAACTTCATCGAGAAGTTTA
CCACAGAGCGGTACTTCTGCCCCAATACCAGATGTAGCATCACATGGTTTCTGAGCTGG
TCCCCTTGCGGAGAGTGTAGCAGGGCCATCACCGAGTTCCTGTCCAGATATCCACACGT
GACACTGTTTATCTACATCGCCAGGCTGTATCACCACGCAGACCCAAGGAATAGGCAGG
GCCTGCGCGATCTGATCAGCTCCGGCGTGACCATCCAGATCATGACAGAGCAGGAGTCC
GGCTACTGCTGGCGGAACTTCGTGAATTATTCTCCTAGCAACGAGGCCCACTGGCCTAG
GTACCCACACCTGTGGGTGCGCCTGTACGTGCTGGAGCTGTATTGCATCATCCTGGGCC
TGCCCCCTTGTCTGAATATCCTGCGGAGAAAGCAGCCCCAGCTGACCTTCTTTACAATC
GCCCTGCAGTCTTGTCACTATCAGAGGCTGCCACCCCACATCCTGTGGGCCACAGGCCT
GAAGTCTGGAGGATCTAGCGGAGGATCCTCTGGCAGCGAGACACCAGGAACAAGCGAGT
CAGCAACACCAGAGAGCAGTGGCGGCAGCAGCGGCGGCAGCGACAAGAAGTACAGCATC
GGCCTGGCCATCGGCACCAACTCTGTGGGCTGGGCCGTGATCACCGACGAGTACAAGGT
GCCCAGCAAGAAATTCAAGGTGCTGGGCAACACCGACCGGCACAGCATCAAGAAGAACC
TGATCGGAGCCCTGCTGTTCGACAGCGGCGAAACAGCCGAGGCCACCCGGCTGAAGAGA
ACCGCCAGAAGAAGATACACCAGACGGAAGAACCGGATCTGCTATCTGCAAGAGATCTT
CAGCAACGAGATGGCCAAGGTGGACGACAGCTTCTTCCACAGACTGGAAGAGTCCTTCC
TGGTGGAAGAGGATAAGAAGCACGAGCGGCACCCCATCTTCGGCAACATCGTGGACGAG
GTGGCCTACCACGAGAAGTACCCCACCATCTACCACCTGAGAAAGAAACTGGTGGACAG
CACCGACAAGGCCGACCTGCGGCTGATCTATCTGGCCCTGGCCCACATGATCAAGTTCC
GGGGCCACTTCCTGATCGAGGGCGACCTGAACCCCGACAACAGCGACGTGGACAAGCTG
TTCATCCAGCTGGTGCAGACCTACAACCAGCTGTTCGAGGAAAACCCCATCAACGCCAG
CGGCGTGGACGCCAAGGCCATCCTGTCTGCCAGACTGAGCAAGAGCAGACGGCTGGAAA
ATCTGATCGCCCAGCTGCCCGGCGAGAAGAAGAATGGCCTGTTCGGAAACCTGATTGCC
CTGAGCCTGGGCCTGACCCCCAACTTCAAGAGCAACTTCGACCTGGCCGAGGATGCCAA
ACTGCAGCTGAGCAAGGACACCTACGACGACGACCTGGACAACCTGCTGGCCCAGATCG
GCGACCAGTACGCCGACCTGTTTCTGGCCGCCAAGAACCTGTCCGACGCCATCCTGCTG
AGCGACATCCTGAGAGTGAACACCGAGATCACCAAGGCCCCCCTGAGCGCCTCTATGAT
CAAGAGATACGACGAGCACCACCAGGACCTGACCCTGCTGAAAGCTCTCGTGCGGCAGC
AGCTGCCTGAGAAGTACAAAGAGATTTTCTTCGACCAGAGCAAGAACGGCTACGCCGGC
TACATTGACGGCGGAGCCAGCCAGGAAGAGTTCTACAAGTTCATCAAGCCCATCCTGGA
AAAGATGGACGGCACCGAGGAACTGCTCGTGAAGCTGAACAGAGAGGACCTGCTGCGGA
AGCAGCGGACCTTCGACAACGGCAGCATCCCCCACCAGATCCACCTGGGAGAGCTGCAC
GCCATTCTGCGGCGGCAGGAAGATTTTTACCCATTCCTGAAGGACAACCGGGAAAAGAT
CGAGAAGATCCTGACCTTCCGCATCCCCTACTACGTGGGCCCTCTGGCCAGGGGAAACA
GCAGATTCGCCTGGATGACCAGAAAGAGCGAGGAAACCATCACCCCCTGGAACTTCGAG
GAAGTGGTGGACAAGGGCGCTTCCGCCCAGAGCTTCATCGAGCGGATGACCAACTTCGA
TAAGAACCTGCCCAACGAGAAGGTGCTGCCCAAGCACAGCCTGCTGTACGAGTACTTCA
CCGTGTATAACGAGCTGACCAAAGTGAAATACGTGACCGAGGGAATGAGAAAGCCCGCC
TTCCTGAGCGGCGAGCAGAAAAAGGCCATCGTGGACCTGCTGTTCAAGACCAACCGGAA
AGTGACCGTGAAGCAGCTGAAAGAGGACTACTTCAAGAAAATCGAGTGCTTCGACTCCG
TGGAAATCTCCGGCGTGGAAGATCGGTTCAACGCCTCCCTGGGCACATACCACGATCTG
CTGAAAATTATCAAGGACAAGGACTTCCTGGACAATGAGGAAAACGAGGACATTCTGGA
AGATATCGTGCTGACCCTGACACTGTTTGAGGACAGAGAGATGATCGAGGAACGGCTGA
AAACCTATGCCCACCTGTTCGACGACAAAGTGATGAAGCAGCTGAAGCGGCGGAGATAC
ACCGGCTGGGGCAGGCTGAGCCGGAAGCTGATCAACGGCATCCGGGACAAGCAGTCCGG
CAAGACAATCCTGGATTTCCTGAAGTCCGACGGCTTCGCCAACAGAAACTTCATGCAGC
TGATCCACGACGACAGCCTGACCTTTAAAGAGGACATCCAGAAAGCCCAGGTGTCCGGC
CAGGGCGATAGCCTGCACGAGCACATTGCCAATCTGGCCGGCAGCCCCGCCATTAAGAA
GGGCATCCTGCAGACAGTGAAGGTGGTGGACGAGCTCGTGAAAGTGATGGGCCGGCACA
AGCCCGAGAACATCGTGATCGAAATGGCCAGAGAGAACCAGACCACCCAGAAGGGACAG
AAGAACAGCCGCGAGAGAATGAAGCGGATCGAAGAGGGCATCAAAGAGCTGGGCAGCCA
GATCCTGAAAGAACACCCCGTGGAAAACACCCAGCTGCAGAACGAGAAGCTGTACCTGT
ACTACCTGCAGAATGGGCGGGATATGTACGTGGACCAGGAACTGGACATCAACCGGCTG
TCCGACTACGATGTGGACCATATCGTGCCTCAGAGCTTTCTGAAGGACGACTCCATCGA
CAACAAGGTGCTGACCAGAAGCGACAAGAACCGGGGCAAGAGCGACAACGTGCCCTCCG
AAGAGGTCGTGAAGAAGATGAAGAACTACTGGCGGCAGCTGCTGAACGCCAAGCTGATT
ACCCAGAGAAAGTTCGACAATCTGACCAAGGCCGAGAGAGGCGGCCTGAGCGAACTGGA
TAAGGCCGGCTTCATCAAGAGACAGCTGGTGGAAACCCGGCAGATCACAAAGCACGTGG
CACAGATCCTGGACTCCCGGATGAACACTAAGTACGACGAGAATGACAAGCTGATCCGG
GAAGTGAAAGTGATCACCCTGAAGTCCAAGCTGGTGTCCGATTTCCGGAAGGATTTCCA
GTTTTACAAAGTGCGCGAGATCAACAACTACCACCACGCCCACGACGCCTACCTGAACG
CCGTCGTGGGAACCGCCCTGATCAAAAAGTACCCTAAGCTGGAAAGCGAGTTCGTGTAC
GGCGACTACAAGGTGTACGACGTGCGGAAGATGATCGCCAAGAGCGAGCAGGAAATCGG
CAAGGCTACCGCCAAGTACTTCTTCTACAGCAACATCATGAACTTTTTCAAGACCGAGA
TTACCCTGGCCAACGGCGAGATCCGGAAGCGGCCTCTGATCGAGACAAACGGCGAAACC
GGGGAGATCGTGTGGGATAAGGGCCGGGATTTTGCCACCGTGCGGAAAGTGCTGAGCAT
GCCCCAAGTGAATATCGTGAAAAAGACCGAGGTGCAGACAGGCGGCTTCAGCAAAGAGT
CTATCCTGCCCAAGAGGAACAGCGATAAGCTGATCGCCAGAAAGAAGGACTGGGACCCT
AAGAAGTACGGCGGCTTCGACAGCCCCACCGTGGCCTATTCTGTGCTGGTGGTGGCCAA
AGTGGAAAAGGGCAAGTCCAAGAAACTGAAGAGTGTGAAAGAGCTGCTGGGGATCACCA
TCATGGAAAGAAGCAGCTTCGAGAAGAATCCCATCGACTTTCTGGAAGCCAAGGGCTAC
AAAGAAGTGAAAAAGGACCTGATCATCAAGCTGCCTAAGTACTCCCTGTTCGAGCTGGA
AAACGGCCGGAAGAGAATGCTGGCCTCTGCCGGCGAACTGCAGAAGGGAAACGAACTGG
CCCTGCCCTCCAAATATGTGAACTTCCTGTACCTGGCCAGCCACTATGAGAAGCTGAAG
GGCTCCCCCGAGGATAATGAGCAGAAACAGCTGTTTGTGGAACAGCACAAGCACTACCT
GGACGAGATCATCGAGCAGATCAGCGAGTTCTCCAAGAGAGTGATCCTGGCCGACGCTA
ATCTGGACAAAGTGCTGTCCGCCTACAACAAGCACCGGGATAAGCCCATCAGAGAGCAG
GCCGAGAATATCATCCACCTGTTTACCCTGACCAATCTGGGAGCCCCTGCCGCCTTCAA
GTACTTTGACACCACCATCGACCGGAAGAGGTACACCAGCACCAAAGAGGTGCTGGACG
CCACCCTGATCCACCAGAGCATCACCGGCCTGTACGAGACACGGATCGACCTGTCTCAG
CTGGGAGGTGACAGCGGCGGGAGCGGCGGGAGCGGGGGGAGCACTAATCTGAGCGACAT
CATTGAGAAGGAGACTGGGAAACAGCTGGTCATTCAGGAGTCCATCCTGATGCTGCCTG
AGGAGGTGGAGGAAGTGATCGGCAACAAGCCAGAGTCTGACATCCTGGTGCACACCGCC
TACGACGAGTCCACAGATGAGAATGTGATGCTGCTGACCTCTGACGCCCCCGAGTATAA
GCCTTGGGCCCTGGTCATCCAGGATTCTAACGGCGAGAATAAGATCAAGATGCTGAGCG
GAGGATCCGGAGGATCTGGAGGCAGCACCAACCTGTCTGACATCATCGAGAAGGAGACA
GGCAAGCAGCTGGTCATCCAGGAGAGCATCCTGATGCTGCCCGAAGAAGTCGAAGAAGT
GATCGGAAACAAGCCTGAGAGCGATATCCTGGTCCATACCGCCTACGACGAGAGTACCG
ACGAAAATGTGATGCTGCTGACATCCGACGCCCCAGAGTATAAGCCCTGGGCTCTGGTC
ATCCAGGATTCCAACGGAGAGAACAAAATCAAAATGCTGTCTGGCGGCTCAAAAAGAAC
CGCCGACGGCAGCGAATTCGAGCCCAAGAAGAAGAGGAAAGTCGGAAGCGGAGCTACTA
ACTTCAGCCTGCTGAAGCAGGCTGGAGACGTGGAGGAGAACCCTGGACCTATGAGCGAG
CTGATCAAGGAGAACATGCACATGAAGCTGTACATGGAGGGCACCGTGAACAACCACCA
CTTCAAGTGCACATCCGAGGGCGAAGGCAAGCCCTACGAGGGCACCCAGACCATGAAGA
TCAAGGTGGTCGAGGGCGGCCCTCTCCCCTTCGCCTTCGACATCCTGGCTACCAGCTTC
ATGTACGGCAGCAAAGCCTTCATCAACCACACCCAGGGCATCCCCGACTTCTTTAAGCA
GTCCTTCCCTGAGGGCTTCACATGGGAGAGAATCACCACATACGAAGACGGGGGCGTGC
TGACCGCTACCCAGGACACCAGCTTCCAGAACGGCTGCATCATCTACAACGTCAAGATC
AACGGGGTGAACTTCCCATCCAACGGCCCTGTGATGCAGAAGAAAACACGCGGCTGGGA
GGCCAACACCGAGATGCTGTACCCCGCTGACGGCGGCCTGAGAGGCCACAGCCAGATGG
CCCTGAAGCTCGTGGGCGGGGGCTACCTGCACTGCTCCTTCAAGACCACATACAGATCC
AAGAAACCCGCTAAGAACCTCAAGATGCCCGGCTTCCACTTCGTGGACCACAGACTGGA
AAGAATCAAGGAGGCCGACAAAGAGACCTACGTCGAGCAGCACGAGATGGCTGTGGCCA
AGTACTGCGACCTCCCTAGCAAACTGGGGCACAGATCTGGTGGTTCTCCCAAGAAGAAG
AGGAAAGTCTAA

### Split System

In some embodiments, the ABE disclosed herein comprises a “split” system, where either the Cas protein, the adenosine deaminase, or both, is split into two constituent parts and can be reconstituted to form a functional protein to execute base editing. A “split Cas9 protein,” “split Cas9,” or “split adenosine deaminase” refers to a Cas9 or adenosine deaminase protein that is provided as an N-terminal fragment and a C-terminal fragment encoded by two separate nucleotide sequences. The polypeptides corresponding to the N-terminal portion and the C-terminal portion of the Cas9 protein or adenosine deaminase can be spliced to form a “reconstituted” Cas9 protein or adenosine deaminase. In some embodiments, the Cas9 protein or adenosine deaminase is divided into two fragments within a disordered region of the protein, e.g., as described in Nishimasu et al., Cell, Volume 156, Issue 5, pp. 935-949, 2014, or as described in Jiang et al. (2016) Science 351: 867-871. PDB file: 5F9R, each of which is incorporated herein by reference.

### Inlaid System

In some embodiments, the ABE system disclosed herein comprises an inlaid system, where the adenosine deaminase is inserted into the Cas9 as exemplified in Example 10. In some embodiments, the disclosure provides a fusion protein comprising a nucleic acid-guided nuclease protein (e.g., a Cas9 or any nuclease disclosed herein) and an inlaid nucleotide deaminase protein domain. In some embodiments, the inlaid nucleotide deaminase protein domain is an inlaid adenosine deaminase protein domain. In some embodiments, the inlaid adenosine deaminase has an amino acid sequence that is at least 80%, 85%, 90%, 95%, 96%, 97%, 98%, or 99% identical to any one of SEQ ID NO: 74, 82, 241, 268-270, 36, 39-47, 49-52, 59, 71, 75-81, 84-87, 94, 116-140, and 220-240, 242.

### EXAMPLES

The compositions and methods disclosed herein are further described in the following examples, which do not limit the scope of the compositions and methods described in the claims.

### Example 1: Designing Adenine Deaminase Variants and Measuring Editing Efficiency by NGS

15 TadA deaminases were selected for targeted mutagenesis to produce adenine deaminase variants with DNA as a substrate. Structure-guided and sequence-guided mutations were introduced into those 15 TadA deaminases and the polynucleotide sequence encoding ecTadA8e in the ABE8e expression vector was replaced with polynucleotide sequences encoding each of the 15 engineered TadA variants to generate a series of 15 ABEs: T7.1, T7.4, T7.5, T7.6, T7.7, T7.8, T7.9, T7.10, T7.11, T7.12, T7.14, T7.15, T7.16, T7.17, and T7.24 (Table 1). Editing activities of the 15 ABEs were measured in HEK293T-GFP cell lines as described in further detail below.

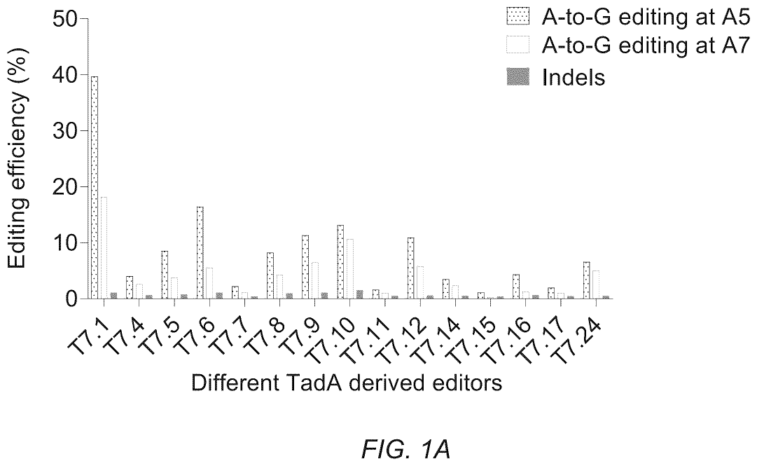

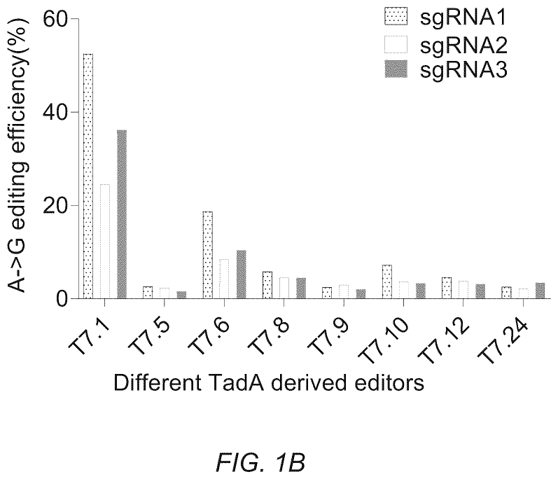

HEK293T-GFP cells (1.5×10⁴) were seeded in a 96-well plate (Corning) 20-24 h before transfection. For on-target editing experiments, cells were transfected with 80 ng of the expression vector encoding the ABE, 40 ng spCas9 gRNA0 (GTT TAG AGT GAG CCA TGT AG (SEQ ID NO: 110)) expression plasmid, and 3.6 μL FuGENE HD Transfection Reagent (E2312) according to the manufacturer's protocols. After 48 hours, genomic DNA of the cells was extracted and analyzed by NGS for editing efficiency. Results for the 15 ABEs are shown in FIG. 1A.

Next, editing efficiency of a subset of 8 of the 15 ABEs (T7.1, T7.5, T7.6, T7.8, T7.9, T7.10, T7.12, T7.24) was measured using additional sgRNAs (sgRNAs 1-3 shown in Table 2). 88 ng of the expression vector encoding each of the 15 ABEs, 24 ng sgRNA1, 24 ng sgRNA2 and 24 ng sgRNA3 expression plasmids were co-transfected into HEK293T cells. After 48 hours, genomic DNA of those cells was extracted and analyzed by NGS for editing efficiency. Results for the 8 ABEs are shown in FIG. 1B and Table 3. A-to-G editing efficiency is calculated as the percentage of reads with A to G mutations in total reads.

These results demonstrate that ABEs comprising TadA variants disclosed herein have low to moderate DNA adenine deamination activities, showing the possibility of evolving new ABEs from TadA orthologs.

TABLE 2
spCas9 sgRNA sequences

| sgRNA name | Sequence | Target gene |
|---|---|---|
| sgRNA1 | CACACACACTTAGAATCTGT (SEQ ID NO: 107) | intergenic |
| sgRNA2 | GCAGAGAGTCGCCGTCTCCA (SEQ ID NO: 108) | intergenic |
| sgRNA3 | CCCGCACCTTGGCGCAGCGG (SEQ ID NO: 109) | PCSK9 |
| sgRNA4 | CCCCACCGTTGAAGAACCAG (SEQ ID NO: 243) | CEACAM16 |
| sgRNA5 | GAGTCCGAGCAGAAGAAGAA (SEQ ID NO: 244) | EMX1 |

TABLE 3
A to G editing efficiency of 8 ABEs

| Base editor | sgRNA1 | sgRNA2 | sgRNA3 |
|---|---|---|---|
| T7.1 | 52.48 | 24.53 | 36.2 |
| T7.5 | 2.62 | 2.3 | 1.61 |
| T7.6 | 18.71 | 8.41 | 10.39 |
| T7.8 | 5.82 | 4.53 | 4.46 |
| T7.9 | 2.45 | 2.99 | 1.99 |
| T7.10 | 7.2 | 3.7 | 3.3 |
| T7.12 | 4.58 | 3.81 | 3.11 |
| T7.24 | 2.51 | 2.11 | 3.41 |

### Example 2: Further Targeted Engineering of TadA Orthologs Generate DNA Deaminases with High Activity

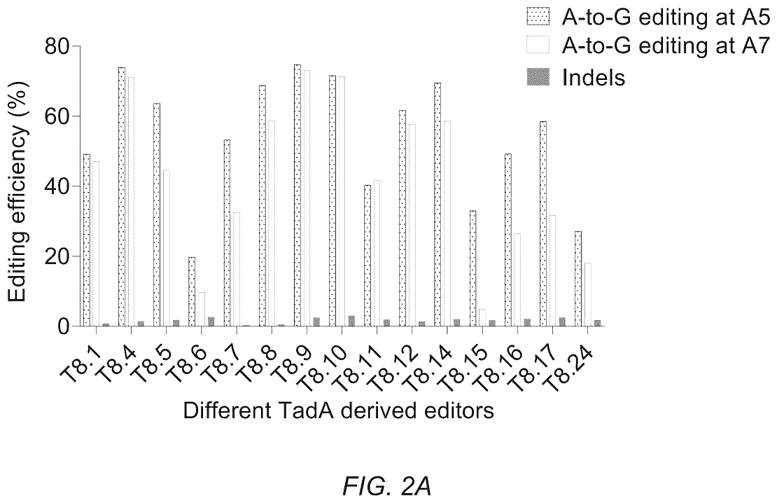

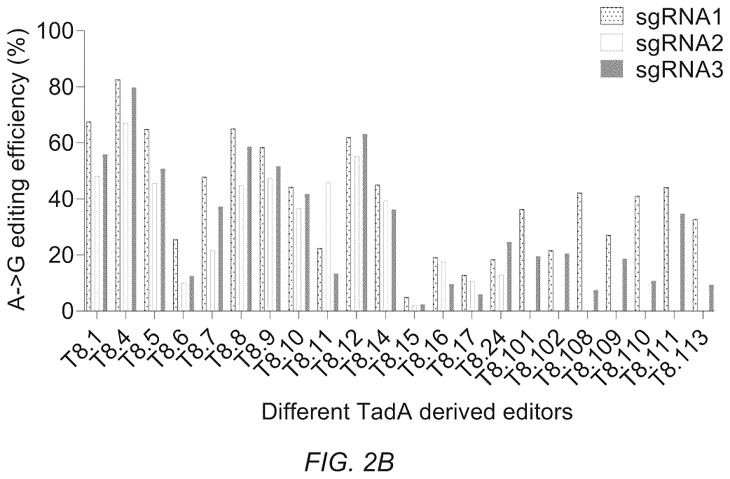

The 15 ABEs described in Example 1 and Table 1 were further engineered with additional functional mutations with the goal of further improving DNA deamination activity. These 15 additional ABE variants were designated T8.1, T8.4, T8.5, T8.6, T8.7, T8.8, T8.9, T8.10, T8.11, T8.12, T8.14, T8.15, T8.16, T8.17, T8.24. The additional 15 ABE variants were tested in HEK293T-GFP cells with sgRNA0 (GTT TAG AGT GAG CCA TGT AG (SEQ ID NO: 110)) according to the protocol described in Example 1. Several of these additional 15 ABE variants exhibited moderate to high A-to-G editing efficiency with top variants exhibited around 75% A-to-G editing efficiency (FIG. 2A).

The additional ABE variants were further characterized using additional sgRNAs (sgRNAs 1-3 shown in Table 2) according to the protocol described in Example 1. ABE variants T8.1, T8.4, T8.5, T8.8, T8.9, T8.10, T8.12, and T8.14 exhibited robust editing across all sites (FIG. 2B and Table 4). A-to-G editing efficiency is calculated as the percentage of reads comprising an A-to-G mutations, out of the total number of reads.

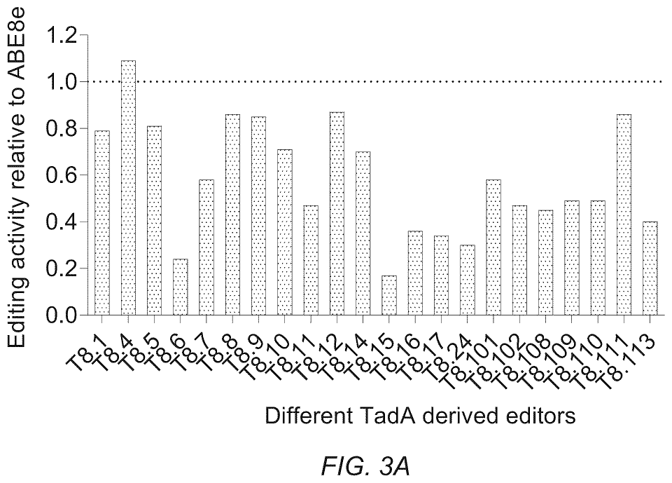

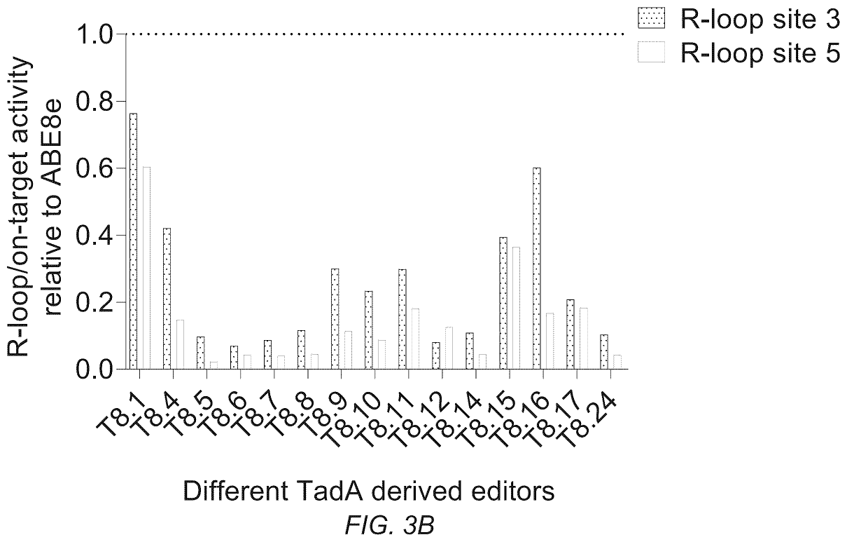

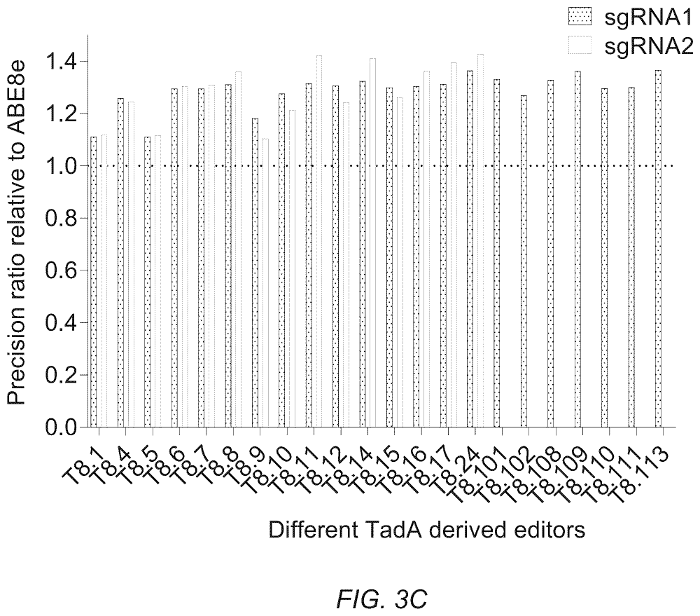

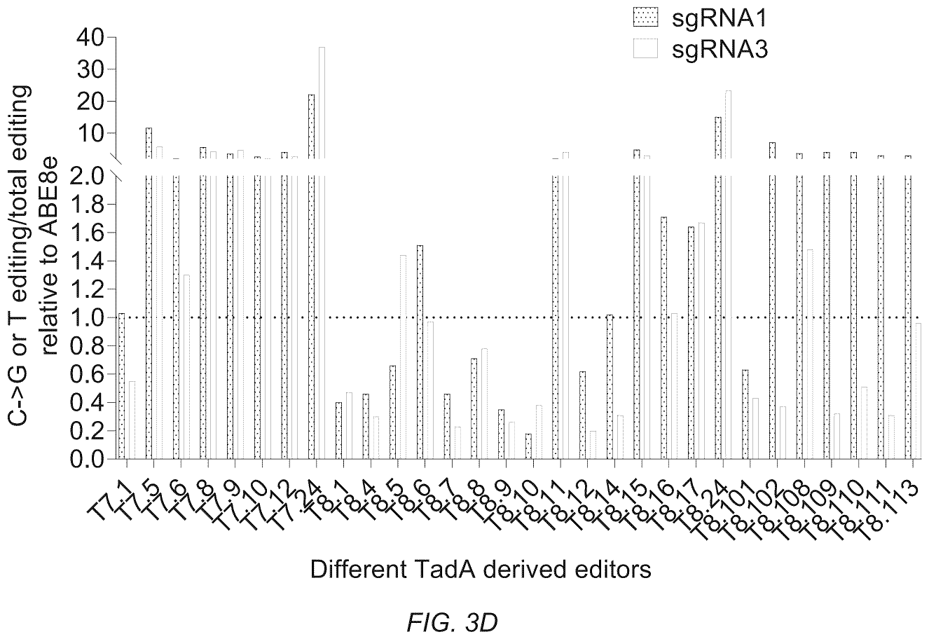

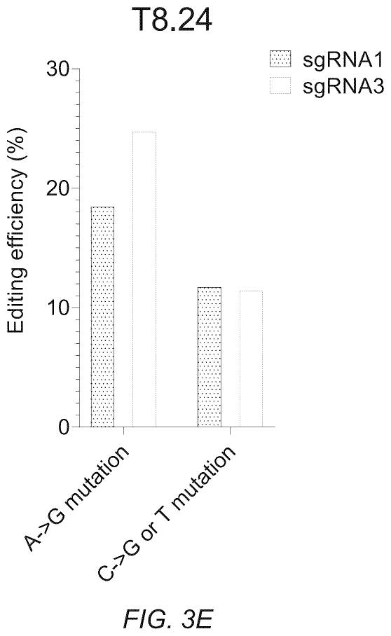

A subset of the ABE variants were further engineered to generate ABE variants T8.101, T8.102, T8.108, T8.109, T8.110, T8.111, T8.113, T8.26, T8.28, T8.37, T8.52, T8.60, T8.61, T8.62, T8.65, T88.13, T88.23, T88.24, T88.30, T88.37, T88.52, T88.54, T88.57, T88.74, T88.84, T8.103, T8.105, T8.112, T8.114 and T8.107. Several of these additional ABE variants exhibited robust editing activities across different sites (FIG. 2B and Table 4). These additional ABE variants were compared with the ABE8e base editor, with several of the engineered ABE variants disclosed herein exhibiting comparable editing efficiency. ABE T8.4 exhibited higher editing efficiency than ABE8e (FIG. 3A and Table 5).

TABLE 4
A to G editing efficiency of further engineered ABEs

| Base editor | sgRNA1 | sgRNA2 | sgRNA3 | sgRNA0 |
|---|---|---|---|---|
| T8.1 | 67.56 | 48.19 | 55.91 | 49.20 |
| T8.4 | 82.59 | 67.07 | 79.79 | 73.99 |
| T8.5 | 64.89 | 45.63 | 50.77 | 63.68 |
| T8.6 | 25.54 | 10.12 | 12.54 | 19.80 |
| T8.7 | 47.87 | 21.69 | 37.3 | 53.28 |
| T8.8 | 65.07 | 44.81 | 58.65 | 68.86 |
| T8.9 | 58.41 | 47.41 | 51.67 | 74.77 |
| T8.10 | 44.22 | 36.71 | 41.82 | 71.66 |
| T8.11 | 22.45 | 45.83 | 13.39 | 41.80 |
| T8.12 | 62.01 | 55.2 | 63.11 | 61.69 |
| T8.14 | 45.01 | 39.3 | 36.24 | 69.52 |
| T8.15 | 4.97 | 2.1 | 2.36 | 33.03 |
| T8.16 | 19.21 | 17.65 | 9.64 | 49.25 |
| T8.17 | 12.92 | 10.65 | 5.95 | 58.53 |
| T8.24 | 18.43 | 12.92 | 24.71 | 27.21 |
| T8.101 | 36.33 | | 19.56 | |
| T8.102 | 21.61 | | 20.49 | |
| T8.108 | 42.22 | | 7.55 | |
| T8.109 | 27.1 | | 18.71 | |
| T8.110 | 41.07 | | 10.78 | |
| T8.111 | 44.16 | | 34.75 | |
| T8.113 | 32.83 | | 9.38 | |
| T8.26 | 13.39 | 4.33 | 10.49 | 10.11 |
| T8.28 | 14.91 | 6.14 | 14.64 | 14.61 |
| T8.37 | 10.51 | | 3.95 | 15.16 |
| T8.52 | 10.47 | | 13.83 | 18.76 |
| T8.60 | 5.08 | | 4.40 | 26.38 |
| T8.61 | 9.95 | | 7.43 | 29.43 |
| T8.62 | 0.86 | | 1.70 | 19.78 |
| T8.65 | 4.09 | | 3.34 | 19.84 |
| T88.13 | 27.87 | | 6.09 | 16.56 |
| T88.23 | 19.80 | | 7.77 | 15.30 |
| T88.24 | 7.29 | | | |
| T88.30 | 1.38 | | | |
| T88.37 | 8.91 | | 7.14 | 8.17 |
| T88.52 | 14.63 | | 6.93 | 14.93 |
| T88.54 | 16.81 | | 4.99 | 13.55 |
| T88.57 | 12.39 | | 2.43 | 10.45 |
| T88.74 | 4.26 | | | |
| T88.84 | 17.42 | | 0.06 | 18.46 |
| T8.103 | 23.73 | | 6.04 | |
| T8.105 | 14.86 | | 5.78 | |
| T8.112 | 30.59 | | 16.68 | |
| T8.114 | 30.45 | | 11.69 | |
| T8.107 | 29.57 | | 16.51 | |

TABLE 5
A-to-G editing efficiency of ABE variants relative to ABE8e

| | A-to-G editing ratio normalized | | | | |
|---|---|---|---|---|---|
| Base | by ABE8e activity in same experiment | | | | Average |
| editor | sgRNA0 | sgRNA1 | sgRNA2 | sgRNA3 | ratio |
| T8.1 | 0.81 | 0.82 | 0.76 | 0.75 | 0.79 |
| T8.4 | 1.21 | 1.01 | 1.06 | 1.08 | 1.09 |
| T8.5 | 1.04 | 0.79 | 0.72 | 0.69 | 0.81 |
| T8.6 | 0.32 | 0.31 | 0.16 | 0.17 | 0.24 |
| T8.7 | 0.87 | 0.58 | 0.34 | 0.50 | 0.58 |
| T8.8 | 1.13 | 0.79 | 0.71 | 0.79 | 0.86 |
| T8.9 | 1.22 | 0.71 | 0.75 | 0.70 | 0.85 |
| T8.10 | 1.17 | 0.54 | 0.58 | 0.56 | 0.71 |
| T8.11 | 0.68 | 0.27 | 0.73 | 0.18 | 0.47 |
| T8.12 | 1.01 | 0.76 | 0.87 | 0.85 | 0.87 |
| T8.14 | 1.14 | 0.55 | 0.62 | 0.49 | 0.70 |
| T8.15 | 0.54 | 0.06 | 0.03 | 0.03 | 0.17 |
| T8.16 | 0.81 | 0.23 | 0.28 | 0.13 | 0.36 |
| T8.17 | 0.96 | 0.16 | 0.17 | 0.08 | 0.34 |
| T8.24 | 0.45 | 0.22 | 0.20 | 0.33 | 0.30 |
| T8.101 | | 0.59 | | 0.60 | 0.59 |
| T8.102 | | 0.35 | | 0.22 | 0.28 |
| T8.108 | | 0.68 | | 0.55 | 0.61 |
| T8.109 | | 0.44 | | 0.31 | 0.38 |
| T8.110 | | 0.66 | | 1.01 | 0.84 |
| T8.111 | | 0.71 | | 0.27 | 0.49 |
| T8.113 | | 0.53 | | 0.13 | 0.33 |
| T7.1 | 0.65 | 0.64 | 0.39 | 0.49 | 0.54 |
| T7.5 | 0.14 | 0.03 | 0.04 | 0.02 | 0.06 |
| T7.6 | 0.27 | 0.23 | 0.13 | 0.14 | 0.19 |
| T7.8 | 0.13 | 0.07 | 0.07 | 0.06 | 0.08 |
| T7.9 | 0.19 | 0.03 | 0.05 | 0.03 | 0.07 |
| T7.10 | 0.21 | 0.09 | 0.06 | 0.04 | 0.10 |
| T7.12 | 0.18 | 0.06 | 0.06 | 0.04 | 0.08 |
| T7.24 | 0.11 | 0.03 | 0.03 | 0.05 | 0.05 |
| T8.26 | 0.17 | 0.16 | 0.07 | 0.14 | 0.13 |
| T8.28 | 0.24 | 0.18 | 0.10 | 0.20 | 0.18 |
| T8.37 | 0.24 | 0.17 | | 0.08 | 0.16 |
| T8.52 | 0.30 | 0.29 | | 0.29 | 0.29 |
| T8.60 | 0.42 | 0.14 | | 0.09 | 0.22 |
| T8.61 | 0.46 | 0.27 | | 0.16 | 0.30 |
| T8.62 | 0.31 | 0.02 | | 0.04 | 0.12 |
| T8.65 | 0.31 | 0.11 | | 0.07 | 0.17 |
| T8.103 | | 0.38 | | 0.18 | 0.28 |
| T8.105 | | 0.24 | | 0.17 | 0.20 |
| T8.112 | | 0.49 | | 0.49 | 0.49 |
| T8.114 | | 0.49 | | 0.34 | 0.42 |
| T8.107 | | 0.48 | | 0.48 | 0.48 |
| T88.13 | 0.26 | 0.45 | | 0.13 | 0.28 |
| T88.23 | 0.24 | 0.32 | | 0.16 | 0.24 |
| T88.24 | | 0.12 | | | 0.12 |
| T88.30 | | 0.02 | | | 0.02 |
| T88.37 | 0.13 | 0.14 | | 0.15 | 0.14 |
| T88.52 | 0.24 | 0.24 | | 0.15 | 0.21 |
| T88.54 | 0.21 | 0.27 | | 0.11 | 0.20 |
| T88.57 | 0.16 | 0.20 | | 0.05 | 0.14 |
| T88.74 | | 0.07 | | | 0.07 |
| T88.84 | 0.29 | 0.28 | | 0.00 | 0.19 |

### Example 3: Characterization of Off-Target Activity and Editing Window of ABEs Comprising TadA Variants

To evaluate unguided single-stranded DNA deamination (unguided DNA off-target) mediated by the ABEs comprising TadA variants, R-loop assays were performed. The R-loop assay is a high-throughput method for assessing sgRNA-independent off-target effects of base editors that leverages the orthogonal R-loops generated by SaCas9 nickase to mimic actively transcribed genomic loci that are more susceptible to deaminases. The SaCas9 nickase is fused with two copies of uracil glycosylase inhibitor (UGI), which serves to amplify the potential for off-target effects which are then detected by the assay. As performed in this example, sgRNA3 provided in Table 2 was used to direct on-target editing and two saCas9 gRNAs provided in Table 6 were used to mimic off-target editing.

TABLE 6
saCas9 gRNAs used in off-target experiments

| SgRNA name | Sequence | Target gene |
|---|---|---|
| saRNA3 | GTGTCAGGTAATGTGCTAAACA (SEQ ID NO: | SSH2 |
| | 113) | |
| saRNA5 | TCTGCTTCTCCAGCCCTGGC (SEQ ID NO: 115) | LINC01509 |

For R-loop experiments, 48 ng of the expression vector encoding each of the base editors, 32 ng SpCas9 guide RNA plasmid (sgRNA3 provided in Table 2), 48 ng nSaCas9-2×UGI plasmid and 32 ng SaCas9 guide RNA plasmid were co-transfected into HEK293T cells in a 96-well plate. After two days, NGS analysis was used to detect off-target deamination at the two dSaCas9 loci.

Results of the orthogonal R-loop assay are shown in FIG. 3B and Table 7. All variants tested showed lower R-loop activity comparable to ABE8e, and several variants show also higher editing efficiency at on-target site.

In order to determine the precision of the editing window of the ABEs disclosed herein, the precision ratio for each ABE was estimated. The precision ratio is calculated by dividing the sum of editing at each editable nucleotide within the classical 4-8 basepair window by the sum of editing at each editable nucleotide within the whole protospacer region (5′ position of protospacer numbered 1). Editors with lower precision are expected to induce more editing events outside of the classical window and are therefore estimated to have a lower precision ratio. Results of precision ratio of the ABEs disclosed herein are shown in FIG. 3C and Table 8. Most variants showed higher precision ratio than ABE8e, indicating they could induce less bystander editing.

In some cases, ABEs can induce C-to-T or G editing, which decreases the editing purity of ABEs as an undesired outcome for therapeutic applications. The impurity ratio for the ABEs disclosed herein was estimated by calculating C-to-T or G editing efficiency/total editing efficiency relative to ABE8e. Results are shown in FIG. 3D and Table 9. ABEs T8.1, 4, 7, 8, 9, 10, 12 exhibited higher editing purity compared ABE8e. ABE variant T8.24, T88.74, T8.26, T8.28, T88.24, T88.30 showed greater than 10-fold higher impurity ratio compared to ABE8e, indicating it can be applied for both ABE and CBE (CABE) applications. Further characterization showed that ABE T8.24 could achieve robust A-to-G and C-to-T editing at the same time at different genomic loci (FIG. 3E and Table 10). Among them, ABE variant T88.74 showed robust C-to-T or G editing with low A-to-G editing across three tested sites, which could be potentially used as a CBE deaminase with high editing purity.

TABLE 7
R-loop assays for new ABE variants relative to ABE8e

| | | R-loop site 3 editing | R-loop site 5 editing |
|---|---|---|---|
| | ON target | efficiency/on-target | efficiency/on-target |
| Base | editing | editing efficiency | editing efficiency |
| editor | efficiency | relative to ABE8e | relative to ABE8e |
| T8.1 | 43.265 | 0.764 | 0.604 |
| T8.4 | 58.209 | 0.421 | 0.147 |
| T8.5 | 30.923 | 0.097 | 0.023 |
| T8.6 | 17.068 | 0.070 | 0.043 |
| T8.7 | 23.776 | 0.087 | 0.040 |
| T8.8 | 28.752 | 0.117 | 0.046 |
| T8.9 | 33.504 | 0.300 | 0.115 |
| T8.10 | 41.943 | 0.233 | 0.087 |
| T8.11 | 7.582 | 0.299 | 0.182 |
| T8.12 | 36.214 | 0.081 | 0.126 |
| T8.14 | 25.042 | 0.109 | 0.045 |
| T8.15 | 2.096 | 0.394 | 0.365 |
| T8.16 | 6.975 | 0.601 | 0.168 |
| T8.17 | 4.682 | 0.208 | 0.184 |
| T8.24 | 15.456 | 0.104 | 0.043 |
| ABE8e | 40.226 | 1.000 | 1.000 |
| T7.1 | 16.034 | 0.099 | 0.090 |
| T7.6 | 25.690 | 0.150 | 0.317 |

TABLE 8
Editing precision of new ABE variants relative to ABE8e

| Base editor | sgRNA1 | sgRNA2 |
|---|---|---|
| T8.1 | 1.111 | 1.118 |
| T8.4 | 1.258 | 1.244 |
| T8.5 | 1.110 | 1.117 |
| T8.6 | 1.295 | 1.304 |
| T8.7 | 1.295 | 1.310 |
| T8.8 | 1.311 | 1.361 |
| T8.9 | 1.181 | 1.104 |
| T8.10 | 1.276 | 1.213 |
| T8.11 | 1.315 | 1.422 |
| T8.12 | 1.306 | 1.243 |
| T8.14 | 1.324 | 1.412 |
| T8.15 | 1.299 | 1.261 |
| T8.16 | 1.304 | 1.363 |
| T8.17 | 1.312 | 1.395 |
| T8.24 | 1.364 | 1.427 |
| T8.101 | 1.331 | |
| T8.102 | 1.269 | |
| T8.108 | 1.328 | |
| T8.109 | 1.362 | |
| T8.110 | 1.296 | |
| T8.111 | 1.300 | |
| T8.113 | 1.366 | |
| T7.1 | 1.296 | 1.326 |
| T7.5 | 1.307 | 1.417 |
| T7.6 | 1.291 | 1.299 |
| T7.8 | 1.336 | 1.406 |
| T7.9 | 1.271 | 1.392 |
| T7.10 | 1.315 | 1.404 |
| T7.12 | 1.330 | 1.401 |
| T7.24 | 1.337 | 1.439 |
| T8.1-Q | 1.422 | |
| T8.4-Q | 1.440 | |
| T8.26 | 1.35 | 1.32 |
| T8.28 | 1.35 | 1.39 |
| T8.37 | 1.21 | |
| T8.52 | 1.01 | |
| T8.60 | 0.74 | |
| T8.61 | 0.78 | |
| T8.62 | 0.46 | |
| T8.65 | 0.90 | |
| T8.103 | 1.29 | |
| T8.105 | 1.36 | |
| T8.112 | 1.32 | |
| T8.114 | 1.36 | |
| T8.107 | 1.40 | |
| T88.13 | 1.23 | |
| T88.23 | 1.21 | |
| T88.24 | 1.22 | |
| T88.30 | 1.19 | |
| T88.37 | 1.22 | |
| T88.52 | 1.21 | |
| T88.54 | 1.20 | |
| T88.57 | 1.22 | |
| T88.74 | 1.17 | |
| T88.84 | 1.21 | |
| ABE8e | 1.000 | 1.000 |

TABLE 9
C-to-G or T editing/total editing (impurity ratio) relative to ABE8e

| Base editor | sgRNA1 | sgRNA3 |
|---|---|---|
| T7.1 | 1.03 | 0.55 |
| T7.5 | 11.64 | 5.77 |
| T7.6 | 2.10 | 1.30 |
| T7.8 | 5.56 | 4.31 |
| T7.9 | 3.55 | 4.69 |
| T7.10 | 2.64 | 2.11 |
| T7.12 | 4.07 | 2.66 |
| T7.24 | 22.09 | 36.91 |
| T8.1 | 0.40 | 0.47 |
| T8.4 | 0.46 | 0.30 |
| T8.5 | 0.66 | 1.44 |
| T8.6 | 1.51 | 0.97 |
| T8.7 | 0.46 | 0.23 |
| T8.8 | 0.71 | 0.78 |
| T8.9 | 0.35 | 0.26 |
| T8.10 | 0.18 | 0.38 |
| T8.11 | 2.07 | 4.10 |
| T8.12 | 0.62 | 0.20 |
| T8.14 | 1.02 | 0.31 |
| T8.15 | 4.79 | 3.00 |
| T8.16 | 1.71 | 1.03 |
| T8.17 | 1.64 | 1.67 |
| T8.24 | 15.03 | 23.26 |
| T8.101 | 0.63 | 0.43 |
| T8.102 | 7.10 | 0.37 |
| T8.108 | 3.63 | 1.48 |
| T8.109 | 4.08 | 0.32 |
| T8.110 | 4.09 | 0.51 |
| T8.111 | 2.99 | 0.31 |
| T8.113 | 2.95 | 0.96 |
| T8.26 | 18.09 | 16.22 |
| T8.28 | 13.73 | 10.62 |
| T8.37 | 5.22 | 1.66 |
| T8.52 | 0.44 | 0.40 |
| T8.60 | 1.50 | 1.48 |
| T8.61 | 0.68 | 0.65 |
| T8.62 | 3.18 | 1.83 |
| T8.65 | 1.25 | 1.26 |
| T8.103 | 7.01 | 1.80 |
| T8.105 | 9.05 | 2.51 |
| T8.112 | 3.75 | 0.60 |
| T8.114 | 3.55 | 0.52 |
| T8.107 | 5.20 | 0.65 |
| T88.13 | 1.68 | 0.87 |
| T88.23 | 1.14 | 0.72 |
| T88.24 | 21.87 | |
| T88.30 | 24.23 | |
| T88.37 | 0.99 | |
| T88.52 | 3.29 | 0.78 |
| T88.54 | 1.66 | 1.44 |
| T88.57 | 1.67 | 2.11 |
| T88.74 | 33.31 | |
| T88.84 | 1.73 | 26.55 |

TABLE 10
A-to-G and C-to-T/G editing of selected ABE variants

| | sgRNA4 | | sgRNA1 | | sgRNA5 | |
|---|---|---|---|---|---|---|
| | A-to-G | C-to-G/T | A-to-G | C-to-G/T | A-to-G | C-to-G/T |
| Base editor | mutation | mutation | mutation | mutation | mutation | mutation |
| T8.24 | 3.46 | 12.89 | 18.43 | 11.71 | 3.42 | 35.15 |
| T8.26 | 2.05 | 3.49 | 4.56 | 4.13 | 0.83 | 16.23 |
| T8.28 | 3.13 | 4.72 | 6.01 | 3.32 | 1.45 | 18.92 |
| T88.24 | 1.37 | 23.15 | 2.32 | 3.02 | 0.74 | 34.05 |
| T88.30 | 0.47 | 6.81 | 0.88 | 1.47 | 0.20 | 20.68 |
| T88.74 | 2.32 | 18.25 | 1.73 | 10.70 | 0.49 | 36.64 |

### Example 4: Further Engineering of ABEs to Enhance Precision

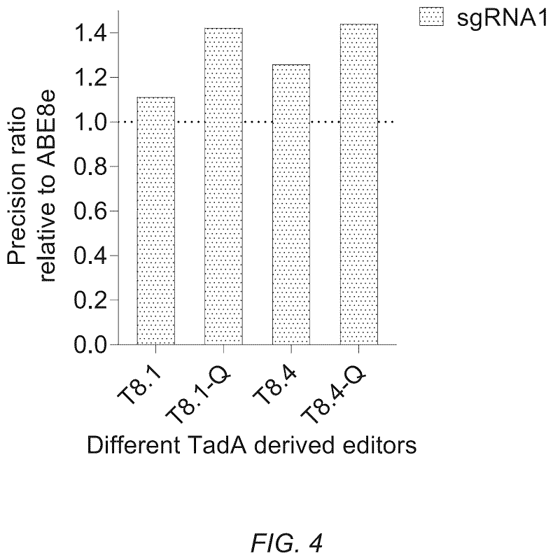

To further improve editing precision, ABEs T8.1 and T8.4 were further engineered, with the resulting ABE variants designated T8.1-Q and T8.4-Q, respectively. The precision ratio for T8.1-Q and T8.4-Q was estimated as described above. Results are shown in FIG. 4 and Table 8. T8.1-Q and T8.4-Q showed improved precision relative to T8.1 and T8.4 and relative to ABE8e.

### Example 5: Further Engineering of TadA Variants to Yield CBE Activity

TadA variants were further engineered in order to explore whether TadA variants could mediate high C-to-T editing in the human genome.

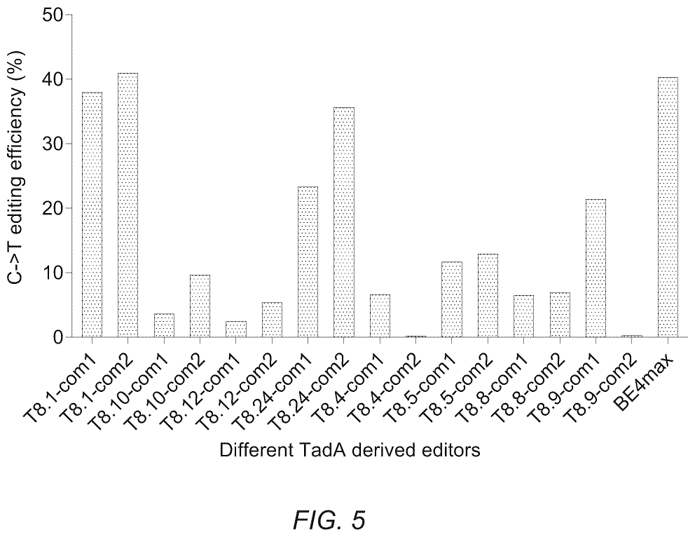

TadA variants with either high A-to-G editing activity or high C-to-G/T editing activity were further engineered to enhance their cytidine deaminase activity. The polynucleotide sequence encoding rAPOBEC1 in BE4max expression vector was replaced with the polynucleotide sequences encoding different TadA variants to generate new CBE variants. Their CBE activities were tested using sgRNA4 (GAG TCC GAG CAG AAG AAG AA (SEQ ID NO: 141)) using the methods described above. Results are shown in FIG. 5 and Table 11. Functional CBEs were obtained with engineered TadA variants and several of the TadA-derived CBEs achieved comparable editing efficiency as BE4max. In general, TadA-derived CBEs induced much less unguided DNA off-target editing and RNA off-target editing compared to rAPOBEC1. The size of most TadA variants are also smaller compared to rAPOBEC1 used by BE4max. Those attributes can be advantageous for use of the TadA variants disclosed herein in CBEs that are safer and smaller alternatives to BE4max.

TABLE 11
C-to-T editing efficiency of TadA-derived CBEs

| | C-to-T |
|---|---|
| | editing |
| Base editor | efficiency |
| T8.1-com1 | 37.96 |
| T8.1-com2 | 40.97 |
| T8.10-com1 | 3.63 |
| T8.10-com2 | 9.68 |
| T8.12-com1 | 2.51 |
| T8.12-com2 | 5.38 |
| T8.24-com1 | 23.32 |
| T8.24-com2 | 35.63 |
| T8.4-com1 | 6.63 |
| T8.4-com2 | 0.22 |
| T8.5-com1 | 11.68 |
| T8.5-com2 | 12.92 |
| T8.8-com1 | 6.50 |
| T8.8-com2 | 6.91 |
| T8.9-com1 | 21.41 |
| T8.9-com2 | 0.25 |
| BE4max | 40.30 |

### Example 6: Evaluating ABE Activity Encoded by mRNA Administered to Huh7 Cells

In order to evaluate base editing activity for ABEs administered as mRNA, mRNA was produced for selected ABE editors and was combined with selected potent sgRNAs (described in Table 12) for activity characterization in Huh7 cells. Huh7 cells were cultured in DMEM media supplemented with 10% fetal bovine serum. Cells were plated at a density of 8000 cells/well in 96-well plates 24 hours prior to transfection. For each sample, 100 ng base editor mRNA and 100 ng end-modified synthesized sgRNA were transfected with Lipofectamine MessengerMAX (ThermoFisher, Cat. LMRNA003) following the manufacturer's protocol. After 72 hours, genomic DNA was harvested from the Huh7 cells and editing efficiency was analyzed by amplicon-seq using NGS. Editing efficiency for each combination of base editor and sgRNA was calculated and is shown in Table 13. All selected base editor variants showed successful editing when administered to cells as mRNA in combination with sgRNAs. T8.1, T8.4, T8.10, T8.12 and T8.111 showed higher average on-target activities relative to the well-known ABE variant ABE8.8 at selected loci.

TABLE 12
sgRNA used for on-target activity
characterization in Huh7 cells (RNA components)

| gRNA | Spacer sequence | SEQ ID NO |
|---|---|---|
| gRNA_h1 | CTAATTACAGCGCGGTGTGG | 245 |
| gRNA_h2 | CAAAAGGTGAAGAAAGAAGT | 246 |
| gRNA_h3 | TCAAAAGGTGAAGAAAGAAG | 247 |
| gRNA_h4 | TCACAGATTATGGCCTGTAT | 248 |
| gRNA_PCSK9 | CCCGCACCTTGGCGCAGCGG | 249 |

TABLE 13
A-to-G Editing Efficiency (%) of ABEs Administered as mRNA in Huh7 Cells

| Base editor | gRNA_h1 | gRNA_h2 | gRNA_h3 | gRNA_h4 | gRNA_PCSK9 | Mean |
|---|---|---|---|---|---|---|
| T7.1 | 51.49 | 24.89 | 18.04 | 16.73 | 55.65 | 33.36 |
| T8.1 | 73.80 | 75.34 | 70.88 | 74.71 | 92.33 | 77.41 |
| T8.4 | 82.46 | 81.10 | 78.17 | 75.19 | 92.18 | 81.82 |
| T8.5 | 87.64 | 43.99 | 58.28 | 26.52 | 77.81 | 58.85 |
| T8.8 | 84.21 | 77.22 | 69.23 | 50.15 | 87.79 | 73.72 |
| T8.10 | 85.73 | 77.61 | 78.95 | 71.34 | 85.46 | 79.82 |
| T8.12 | 87.58 | 76.32 | 77.67 | 61.19 | 89.80 | 78.51 |
| T8.111 | 86.44 | 83.34 | 85.08 | 64.72 | 90.51 | 82.02 |
| T8.112 | 82.35 | 64.23 | 77.12 | 46.11 | 77.04 | 69.37 |
| T8.113 | 79.18 | 14.41 | 27.64 | 17.52 | 64.99 | 40.75 |
| ABE8.8 | 71.46 | 71.10 | 62.15 | 69.96 | 88.60 | 72.65 |

### Example 7: Evaluating ABE Activity Encoded by mRNA Administered to Primary Human Hepatocytes (PHH)

Editing activity of selected ABEs administered as mRNA and in combination with selected sgRNAs was further evaluated in primary human hepatocytes (PHH). PHHs were cultured per the manufacturer's protocol. Cells were thawed and resuspended in hepatocyte thawing medium with supplements followed by centrifugation at 100 g for 10 minutes for concentration. The supernatant was discarded and the pelleted cells were resuspended in hepatocyte plating medium plus supplement. Cells were counted and plated on Bio-coat collagen I coated 48-well plates (ThermoFisher, Cat. 877272) at a density of 132,000 cells/well. Plated cells were allowed to settle and adhere for 4-6 hours in a tissue culture incubator at 37° C. and 5% CO₂ atmosphere. Each well was transfected with 250 ng base editor mRNA and 250 ng end-modified sgRNA using RNAiMax according to manufacturer's protocol. After 24 hours, the culture medium was changed for fresh medium. 72 hours after transfection, genomic DNA were extracted from cells and editing efficiency was analyzed by amplicon-seq using NGS. The editing efficiencies achieved in PHH for each combination of base editor and sgRNA are shown in Table 14. All selected variants showed successful editing in PHH when delivered as mRNA in combination with sgRNAs.

TABLE 14
A-to-G Editing Efficiency (%) of
ABEs Administered as mRNA in PHHs

| Based | | | | | |
|---|---|---|---|---|---|
| editor | gRNA_h1 | gRNA_h2 | gRNA_h3 | gRNA_h4 | gRNA_PCSK9 |
| T8.4 | 44.14 | 40.94 | 42.36 | 23.21 | 72.49 |
| T8.12 | 30.99 | 22.89 | 31.93 | 14.44 | 58.82 |
| T8.111 | 39.71 | 41.16 | 37.05 | 18.84 | 64.91 |

### Example 8: Evaluating ABE Activity in Humanized Mice Model

The editing efficacy of selected base editors was further evaluated using a humanized PCSK9 mouse model with mouse Pcsk9 gene replaced by human PCSK9 gene. A guide RNA (gRNA_PCSK9, detailed in Table 12, SEQ ID NO: 249) was designed specifically to disrupt the human PCSK9 gene and employed in the experiment. The mRNA and modified gRNA (Finn et al., 2018) were combined and encapsulated into lipid nanoparticles (LNPs), following the methodology outlined in patent application WO/2023/185697A2. These formulated LNPs were then administered to humanized mice through tail vein injections at varying doses (0.3/0.6/1/3 mg/kg).

After a 1 or 2-week period, mouse liver and blood samples were collected for analysis. DNA editing in the liver and PCSK9 protein levels in the blood were evaluated. These results demonstrated a higher DNA editing efficiency and PCSK9 protein knock-down efficacy in mice dosed with T8.4 relative to those dosed with ABE8.8 across all dosage ranges. In-laid design of T8.4 also achieved up to 40% editing in liver at 1 mg/kg (mpk) when delivered by LNP. Another two leads T8.1 ABE and T8.111 ABE achieved >50% editing at 3 mpk (shown in Table 15).

TABLE 15
DNA Editing Efficiency (%) and PCSK9 Protein Reduction
Achieved by ABEs in a Humanized PCSK9 Mouse Model

| | | DNA Editing | PCSK9 Protein |
|---|---|---|---|
| | Dose | Efficiency | Reduction |
| Editor | (mpk) | (%) | (%) |
| T8.4 ABE | 1 | 59.18 | 97.15 |
| ABE8.8 | 1 | 46.46 | 90.38 |
| T8.4 ABE-Inlaid | 1 | 40.36 | 65.17 |
| T8.4 ABE | 0.6 | 51.14 | 88.26 |
| ABE8.8 | 0.6 | 36.08 | 72.18 |
| T8.4 ABE-Inlaid | 0.6 | 21.48 | 65.73 |
| T8.4 ABE | 0.3 | 25.08 | 43.81 |
| T8.4 ABE | 0.3 | 22.03 | 68.58 |
| T8.4 ABE | 0.3 | 24.64 | 48.74 |
| ABE8.8 | 0.3 | 13.78 | −7.69 |
| ABE8.8 | 0.3 | 8.03 | 15.44 |
| ABE8.8 | 0.3 | 8.34 | 19.34 |
| T8.4 ABE-Inlaid | 0.3 | 6.37 | 11.00 |
| T8.4 ABE | 3 | 70.51 | 98.46 |
| ABE8.8 | 3 | 69.56 | 97.82 |
| T8.1 ABE | 3 | 50.81 | 80.18 |
| T8.111 ABE | 3 | 63.26 | 93.56 |

### Example 9: Deaminase T8.4 Shows Activity Combined with Other Programmable DNA Binding Proteins

To evaluate the potential of T8.4 deaminase (SEQ ID NO: 74) in combination with other programmable DNA binding domains, dead LbCpf1, ISAam1-D363A(TnpB) and enIscB-D61A(IscB) were each fused with the T8.4 deaminase sequence at their N terminus. Following transfection with T8.4-fused base editor (80 ng) and gRNA (40 ng) expression plasmids (gRNA sequence in Table 16), as per the provided instructions mentioned above, genomic DNA was collected from these cells after a 72-hour incubation period for NGS analysis to determine DNA editing efficiencies. The results are shown in Table 17.

TABLE 16
sgRNAs Used for On-target Activity Characterization

| gRNA | Spacer sequence | SEQ ID NO | Target gene |
|---|---|---|---|
| gRNA_t1 | GGCTGGCAGGCTCCTTGTTC | 250 | PGK1 |
| gRNA_t2 | GCAAATCAGCATGTTCCTCA | 251 | AGBL1 |
| gRNA_i1 | AGTGGAAGAAGGAGAT | 252 | ALDH1A3 |
| gRNA_i2 | ATCTTCACCAGGAAGC | 253 | PCSK9 |
| gRNA_c1 | TGACTCACACTCACCCAAAA | 254 | HSD17B13 |
| gRNA_c1 | CTGGAACTGTAAGAGAATTA | 255 | HSD17B13 |

TABLE 17
C-to-T Editing Efficiency of T8.4 in Combination
with Different Programmable DNA Binding Proteins

| Base editor | gRNA | Editing efficiency (%) |
|---|---|---|
| T8.4_TnpB-D363A | gRNA_t1 | 8.11 |
| | gRNA_t2 | 6.13 |
| T8.4-IscB-D61A | gRNA_il | 14.69 |
| | gRNA_i2 | 10.63 |
| T8.4-LbCpf1 | gRNA_cl | 12.12 |
| | gRNA_cl | 5.95 |

### Example 10: Evaluating Editing Activity and Specificity of T8.4 Inlaid Variants

To test whether inlaid variants of the deaminases disclosed herein can improve the specificity of the T8.4 deaminase in the context of base-editing, wherein the deaminase is inlaid into a location within a nuclease protein, the deaminase variant T8.4 was inserted into nCas9 at different sites (detailed in Table 18) by a 20 amino acid linker (Linker: GGSGGSGGSGGSGGSGGSGG (SEQ ID NO: 256); Linker2: GSSGSETPGTSESATPESSG (SEQ ID NO: 257)) or a GGS/GSS linker. On-target and R-loop assays for those inlaid variants were conducted in the same manner as described in Example 2 and 3. The sgRNAs for on and off-target analysis are listed in Table 19 and Table 20. The results of these experiments are shown in Table 21 and Table 22. In particular, Inliad-1, Inlaid-2, Inlaid-6, Inlaid-7, Inalid-9 and Inlaid-10 showed comparable editing efficiency when compared with the original T8.4, while the off-target editing was lower.

TABLE 18
Design of T8.4 Inlaid Variants

| Base editor | Architecture |
|---|---|
| Inlaid-1 | Use Linker1-T8.4-Linker2 to replace 1048-1063 of Cas9 |
| Inlaid-2 | insert Linker1-T8.4-linker2 between R535 and K536 |
| Inlaid-3 | insert Linker1-T8.4-linker2 between Q768 and T769 |
| Inlaid-4 | insert Linker1-T8.4-linker2 between S909 and E910 |
| Inlaid-5 | insert Linker1-T8.4-linker2 between P1249 and E1250 |
| Inlaid-6 | insert Linker1-T8.4-linker2 between 11029 and G1030 |
| Inlaid-7 | insert Linker1-T8.4-linker2 between E1068 and T1069 |
| Inlaid-8 | insert Linker1-T8.4-linker2 between 11022 and A1023 |
| Inlaid-9 | insert GGS-T8.4-GSS between P1249 and E1250 |
| Inlaid-10 | insert Linker1-T8.4 between P1249 and E1250 |

TABLE 19
spCas9 sgRNA sequences

| sgRNA | Sequence | Target gene |
|---|---|---|
| sgRNA4 | GACAAACCAGAAGCCGCTCC (SEQ ID NO: 258) | CTNNB1 |
| sgRNA5 | GTTCACACCCATGACGAACA (SEQ ID NO: 259) | GAPDH |
| sgRNA3 | CCCGCACCTTGGCGCAGCGG (SEQ ID NO: 109) | PCSK9 |

TABLE 20
saCas9 gRNAs used in off-target experiments

| sgRNA name | Sequence | Target gene |
|---|---|---|
| Sa-gRNA2 | ATTTACAGCCTGGCCTTTGGGG (SEQ ID NO: 260) | GAPDH |
| Sa-gRNA4 | GGTGGAGGAGGGTGCATGGGGT (SEQ ID NO: 261) | intergenic |
| Sa-gRNA5 | TCTGCTTCTCCAGCCCTGGC (SEQ ID NO: 115) | LINC01509 |

TABLE 21
A-to-G Editing Efficiency of T8.4 and its Inlaid Variants

| Base editor | sgRNA4 | sgRNA5 | sgRNA3 | mean |
|---|---|---|---|---|
| T8.4 | 57.37 | 50.22 | 37.26 | 48.29 |
| Inlaid-1 | 56.48 | 46.40 | 35.57 | 46.15 |
| Inlaid-2 | 49.91 | 30.63 | 39.60 | 40.05 |
| Inlaid-3 | 41.68 | 8.00 | 8.79 | 19.49 |
| Inlaid-4 | 41.04 | 19.18 | 17.08 | 25.77 |
| Inlaid-5 | 48.72 | 29.08 | 34.52 | 37.44 |
| Inlaid-6 | 57.19 | 39.19 | 37.43 | 44.60 |
| Inlaid-7 | 56.63 | 49.43 | 34.99 | 47.02 |
| Inlaid-8 | 52.04 | 35.23 | 29.06 | 38.77 |
| Inlaid-9 | 60.84 | 40.44 | 39.57 | 46.95 |
| Inlaid-10 | 53.01 | 37.86 | 33.02 | 41.30 |

TABLE 22
Ratio of R-loop Editing to On-target Editing for
Engineered inlaid Variants Relative to T8.4

| | Sa2 R-loop | Sa4 R-loop | Sa5 R-loop | |
|---|---|---|---|---|
| | editing/on- | editing/on- | editing/on- | |
| | target editing | target editing | target editing | |
| Base | efficiency | efficiency | efficiency | |
| editor | relative to T | relative to T | relative to T | Mean |
| T8.4 | 1.00 | 1.00 | 1.00 | 1.00 |
| Inlaid-1 | 0.10 | 0.15 | 0.25 | 0.17 |
| Inlaid-2 | 0.46 | 1.02 | 0.60 | 0.69 |
| Inlaid-3 | 0.31 | 0.72 | 0.84 | 0.62 |
| Inlaid-4 | 0.17 | 0.77 | 0.30 | 0.42 |
| Inlaid-5 | 0.23 | 0.22 | 0.31 | 0.26 |
| Inlaid-6 | 0.20 | 0.42 | 0.46 | 0.36 |
| Inlaid-7 | 0.25 | 0.77 | 0.67 | 0.56 |
| Inlaid-8 | 0.22 | 0.26 | 0.62 | 0.37 |
| Inlaid-9 | 0.17 | 0.39 | 0.29 | 0.28 |
| Inlaid-10 | 0.20 | 0.73 | 0.46 | 0.46 |

### Example 12: Improve T8.4 Deaminase Activity Through Directed Evolution

To augment the activity of T8.4, mutations were intentionally introduced into its sequence and subjected to directed evolution. Two distinct methods were employed to generate the mutational library: an NNK library encompassing 40 residues of T8.4, and an error-prone PCR library using T8.4 as the template and with a mutation frequency ranging from 1 to 3 mutations per sequence. The experimental system utilized three plasmids: a selection plasmid that expressed mutated Kanamycin resistance gene (Kan^{R}-Q4*W15*D208N), featuring three inactivation mutations, a sgRNA expression plasmid that expressed three sgRNAs targeting the mutated sites (Kan-sgRNA 1-3 in Table 23); and an editor plasmid that expressed the T8.4-dCas9 library. Library members capable of effecting C-G to T-A edits at all three sites simultaneously would restore Kanamycin resistance, thereby enabling survival upon challenge with Kanamycin.

In conducting the experiment, E. coli strain DH10B, co-transformed with the selection plasmid and sgRNA expression plasmid, was rendered electrocompetent. Subsequently, 100-200 ng of the Editor plasmid was electroporated into DH10B cells. The bacteria were then recovered in 10 ml of LB medium before inoculation into 20 ml of LB medium supplemented with three antibiotics for plasmid maintenance (50 μg/mL Chloramphenicol, 150 μg/mL Spectinomycin and 100 μg/mL Ampicillin). Additionally, 1% Arabinose was introduced to induce the expression of T8.4-dCas9 library. The bacteria were cultured for an additional 16 hours before being plated onto bioassay dishes containing 1.5% agar-2×YT, the plasmid maintenance antibiotic, and various concentrations of Kanamycin (ranging from 64 to 256 μg/mL). Parallel experiments were conducted on two separate plates. Following incubation at 37° C. overnight, 120 surviving colonies from one plate were individually amplified by PCR and subsequently subjected to Sanger sequencing. Surviving colonies from the other plate were pooled together and subjected to plasmid extraction, defined as post-selection sample. Bacteria without selection were also isolated as a pre-selection control. The T8.4 library genes were then amplified from these extracted plasmids and subsequently subjected to Next-generation sequencing for analysis.

TABLE 23
Target sites of Kanamycin resistance gene

| | Inactivating | Position of target | Corresponding |
|---|---|---|---|
| Target site | mutation | A/C in protospacer | sgRNA |
| ATCTTATTCGATCATGCGAA | Q4* | 6 | Kan-sgRNA1 |
| (SEQ ID NO: 264) | | | |
| GCTTAGGTGGAGCGCCTATT | W15* | 5 | Kan-sgRNA2 |
| (SEQ ID NO: 265) | | | |
| TTCATTAACTGTGGCCGGCT | D208N | 7 | Kan-sgRNA3 |
| (SEQ ID NO: 266) | | | |
| GACTAGGCACAACAGACAAT | W24* | 5 | Kan-sgRNA4 |
| (SEQ ID NO: 267) | | | |

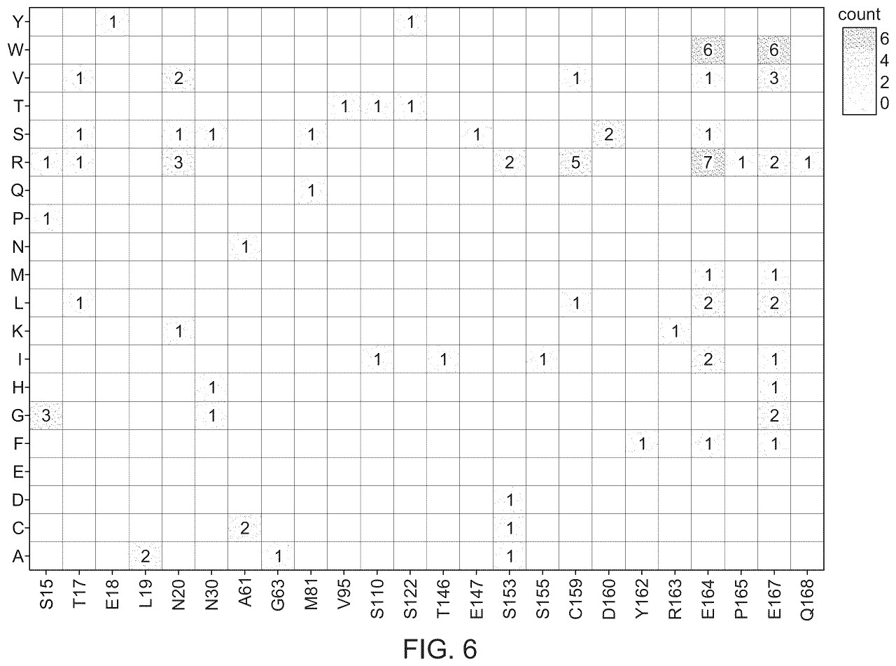

The Sanger results of surviving colonies are shown in FIG. 6. E164, E167, C159, N20, S153, S15 are mutation hotspots.

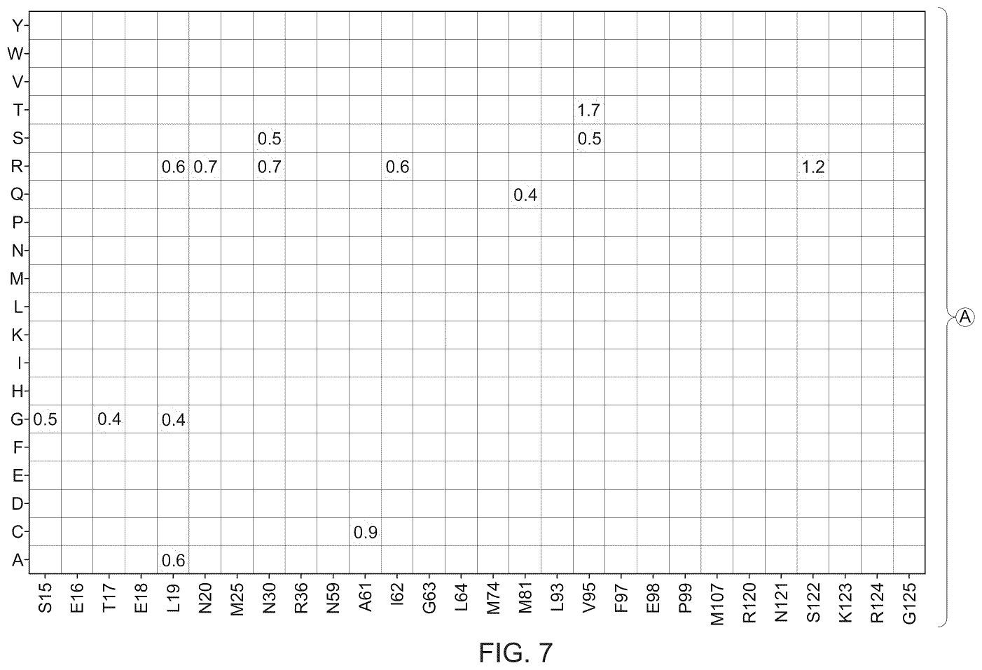

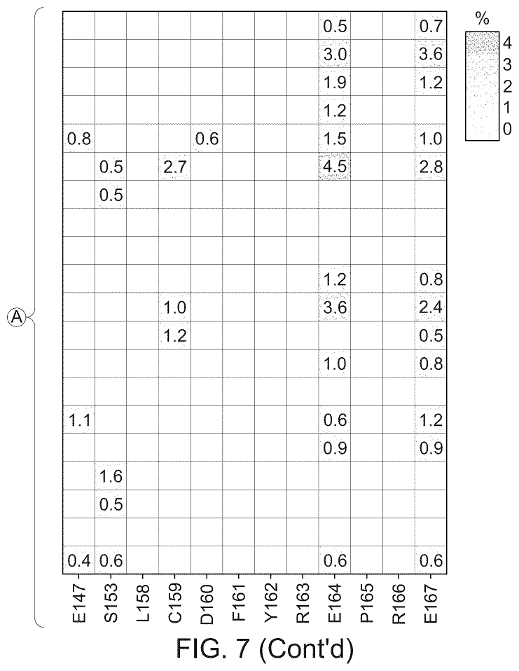

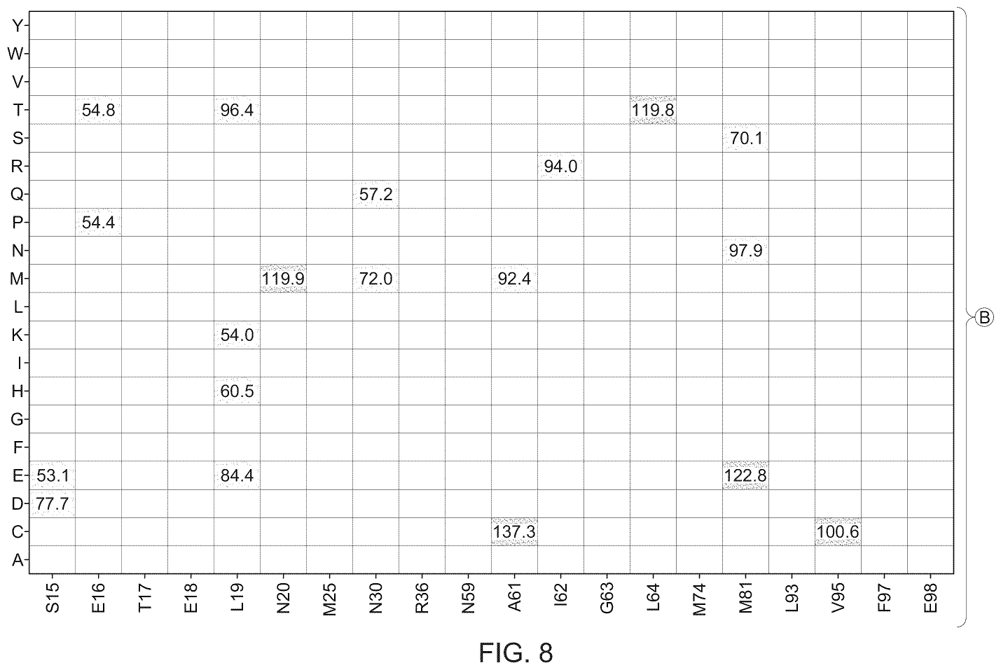

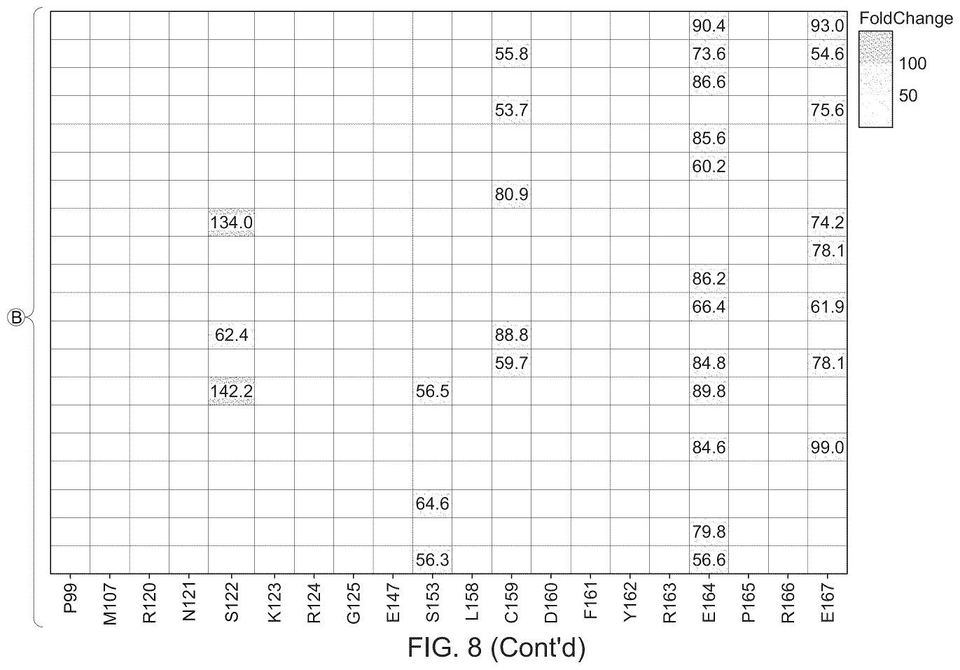

For the NGS analysis, the percentage of mutations present in post-selection samples was calculated for each position by dividing the number of reads containing mutations by the total number of reads. The top 50 values are displayed in FIG. 7. Additionally, the fold change of enrichment was calculated by dividing the percentage of survived mutations in post-selection samples by that in the pre-selection controls, The top 50 values are displayed in FIG. 8.

A second round of evolution was conducted using the combinatorial library of emerged mutations with KanR−Q4*W15*W24*D208N, applying higher selection pressure. The combinatorial library was generated through DNA shuffling. The directed evolution was conducted as described previously. The T8.4 library genes of colonies from pre-selection library and the post-selection library were sequenced via Sanger sequencing. By comparing the mutation ratios before and after selection, enrichment of S15G, S153R, E164V, E164L, and E167W was observed (Shown in Table 24).

TABLE 24
Mutation counts before and after selection

| Position | 15 | 20 | 153 | 159 | 164 | 167 |
|---|---|---|---|---|---|---|
| Residue | S/G | N/RKS | S/R | C/R | E/GV*WLKRM | E/W |
| Count of pre-selection | 11/19 | 9/4/9/8 | 20/10 | 14/16 | 2/1/6/1/5/4/2/4/5 | 15/15 |
| Count of post-selection | 13/28 | 12/7/8/14 | 4/37 | 41/0 | 0/1/12/2/2/19/1/1/3 | 13/28 |

### Example 13: Validation of Beneficial Mutations in E. coli

To validate the editing efficiency of enriched mutations in the two rounds of directed evolution, the single or combinational mutations was cloned back to the Editor plasmid. 50 ng Editor plasmid was transformed into 20 μL of chemically competent TOP10 cells together with 50 ng Target plasmid (selection plasmid Kan^{R}-Q4*W15*D208N in Example 12) and 50 ng corresponding sgRNA expression plasmid (Kan-sgRNA 1-3 in Table 23). Bacteria were recovered in 1 mL of LB medium at 37° C. with shaking for 1 h. Then another 1 ml of LB medium containing selection plasmid maintenance antibiotic (ampicillin, spectinomycin and chloramphenicol) and 1% arabinose was added. Bacteria were grown at 37° C. with shaking for another 16 h. The resulting bacterial culture was subjected to plasmid extraction. The 3 sgRNA targeting sites were then amplified from these extracted plasmids and subsequently subjected to Next-generation sequencing. As shown in Table 25, all of the tested variants showed higher editing efficiency when compared with the original T8.4. And the combination of some mutations exhibited more significant and pronounced effects on editing efficiency.

TABLE 25
A-to-G Editing Efficiency of T8.4 and its Variants in E.coli

| | Kan- | Kan- | Kan- | |
|---|---|---|---|---|
| Base editor | sgRNA1 | sgRNA2 | sgRNA3 | Mean |
| T8.4 | 11.65 | 13.32 | 20.10 | 15.02 |
| T8.4-S155R | 52.00 | 60.00 | 47.22 | 53.07 |
| T8.4-C159R | 49.38 | 41.05 | 45.23 | 45.22 |
| T8.4-D12H | 18.42 | 23.68 | 14.29 | 18.80 |
| T8.4-N20H | 20.00 | 16.67 | 17.86 | 18.17 |
| T8.4-H21D | 34.90 | 28.66 | 29.07 | 30.88 |
| T8.4-E167R | 37.31 | 39.53 | 40.57 | 39.14 |
| T8.4-E164R-E167W | 41.25 | 40.32 | 72.21 | 51.26 |
| T8.4-E164W-E167W | 41.53 | 38.52 | 67.40 | 49.15 |
| T8.4-E164W-E167W-C159R | 53.03 | 60.32 | 77.74 | 63.70 |
| T8.4-S155R-E167R | 41.03 | 51.02 | 52.41 | 48.15 |
| T8.4-S155R-H21D-E167R | 53.87 | 65.41 | 64.58 | 61.29 |
| T8.4-S153R-E164V-E167W | 57.13 | 72.85 | 89.60 | 73.19 |
| T8.4-S153R-E164L-E167W | 49.10 | 58.68 | 83.24 | 63.68 |
| T8.4-S15G-S153R-E164V- | 57.43 | 72.18 | 88.66 | 72.76 |
| E167W | | | | |
| T8.4-S15G-S153R-E164L- | 55.43 | 61.99 | 84.90 | 67.44 |
| E167W | | | | |
| T8.4-N20S-S153R-E164L- | 53.88 | 57.41 | 83.35 | 64.88 |
| E167W | | | | |

### Example 14: Validation of Beneficial Mutations in Mammalian Cells (mRNA+sgRNA Library) sgRNA Library

To systematically evaluate the editing performance of T8.4 variants in mammalian cells, we prepared a library wherein more than 10,000 target sites were individually paired with their cognate sgRNAs. The paired sgRNA-target library was cloned into To12 transposon plasmid. To establish a stable expression of the library, HEK293T cells were seeded into two 6-well plates at a density of 1×10⁶ cells per well. For each well, 50 ng of the library plasmid and 250 ng of the To12 transposase enzyme plasmid were transfected using FuGENE HD Transfection Reagent (E2312) according to the manufacturer's protocols. Two days after transfection, cells were harvested and puromycin was added to select for cells containing the library. After 2-3 weeks of selection, cell lines with stable To12-mediated genomic integration were generated. To evaluate the editing performance, the mRNA of the variants was produced. HEK293T library cells (1×10⁵) were seeded in a 24-well plate (Corning) 20-24 h before transfection. 400 ng T8.4 variant mRNA was transfected into cells using Lipofectamine MessengerMAX (ThermoFisher, Cat. LMRNA003) following the manufacturer's protocol. After 72 hours, genomic DNA of the cells was extracted and analyzed by NGS for editing efficiency. The sgRNA detected in all samples were defined as valid sgRNAs. The A-to-G editing efficiency of these paired target sites was analyzed.

To quantify the potency of the variants, the mean editing efficiency value of the strong T8.4 editing window (positions edited to equal to or more than 40% of the peak editing rate, 3-8 for T8.4) was calculated. As shown in Table 26, the mean editing efficiency of many engineered variants was higher than original T8.4.

TABLE 26
A-to-G Editing Efficiency of T8.4 and its Variants in library cells

| | Average editing efficiency within |
|---|---|
| Base editor | strong editing window (3-8) |
| T8.4 | 49.50 |
| T8.4-E164L | 52.71 |
| T8.4-E164R | 57.01 |
| T8.4-E164W | 57.04 |
| T8.4-E167W | 57.31 |
| T8.4-C159R | 54.52 |
| T8.4-S155R | 52.76 |
| T8.4-E167R | 53.08 |
| T8.4-E164R-E167W | 58.81 |
| T8.4-E164W-E167W | 57.65 |
| T8.4-E164W-E167W-C159R | 62.76 |
| T8.4-S153R-E164V-E167W | 55.88 |
| T8.4-S153R-E164L-E167W | 59.37 |
| T8.4-S15G-S153R-E164V-E167W | 61.26 |
| T8.4-S15G-S153R-E164L-E167W | 58.90 |
| T8.4-N20S-S153R-E164L-E167W | 58.60 |

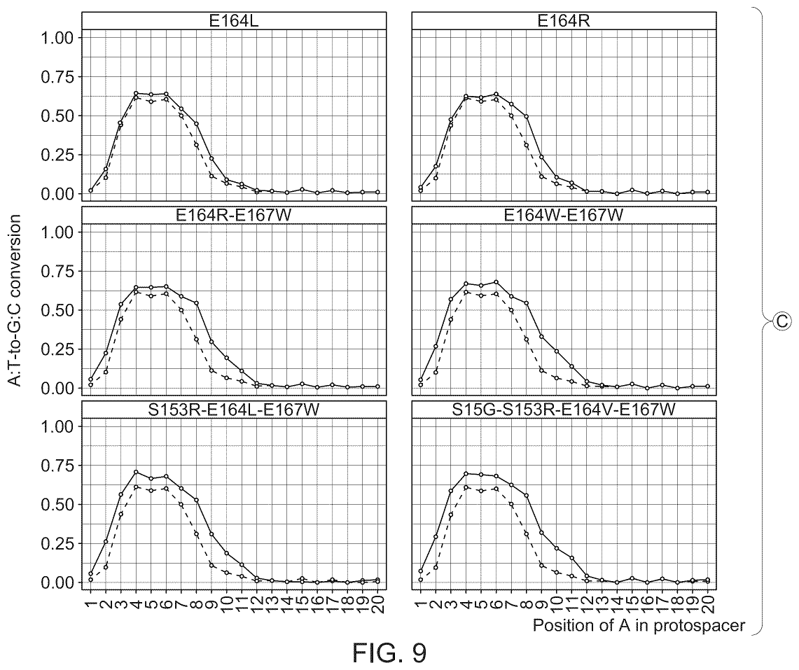

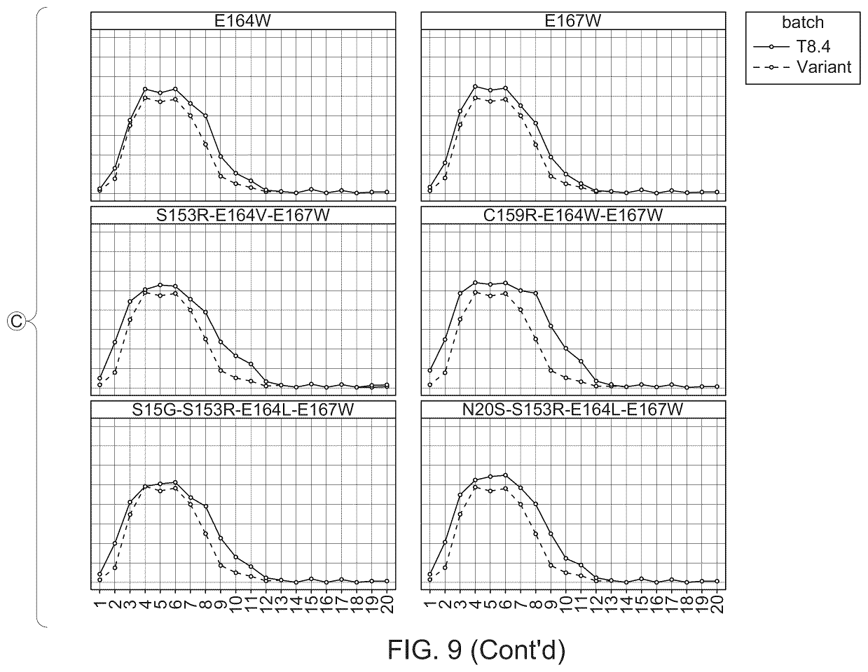

The overall editing results for some ABEs are shown in FIG. 9. When compared with T8.4, some variants showed similar editing window but higher efficiency. Some variants showed broader window and higher potency.

### Example 15: Improve T8.12 Deaminase Activity Through Directed Evolution

To augment the activity of T8.12, a site-saturation mutagenesis library was synthesized and cloned into the editor plasmid backbone. The directed evolution was performed in a similar way as in Example 12. The percentage of mutations in post-selection samples are displayed in Table 27. Three mutations: E107Y, E107K and V104M-E107Y were validated in bacteria as in Example 13. As shown in Table 28 below, all of the three variants showed higher editing efficiency when compared with T8.12.

TABLE 27
Percentage of mutations identified in survival colonies

| Mutation | Percentage |
|---|---|
| E107Y | 21.23 |
| E107K | 19.59 |
| 147Q | 10.60 |
| E57Y | 6.63 |
| V104L | 4.16 |
| R105G | 3.72 |
| E57Y | 3.65 |
| V86D | 3.30 |
| A137V; Q159W | 3.04 |
| R18A | 3.00 |
| V104M; E107Y | 2.76 |
| E57K | 2.28 |
| G113Y | 2.24 |
| V101R | 2.22 |
| G29M | 1.98 |
| A60Q | 1.58 |
| H149L | 1.51 |
| A60D | 1.42 |
| V67P | 1.38 |
| V33S | 1.24 |
| N117V | 1.20 |
| A54P | 1.19 |
| V104M | 1.14 |

TABLE 28
A-to-G Editing Efficiency of T8.12 and its Variants in E. coli

| Base editor | KAN-sgRNA1 | KAN-sgRNA2 | KAN-sgRNA3 | Mean |
|---|---|---|---|---|
| T8.12 | 13.02 | 8.29 | 12.18 | 11.16 |
| T8.12-E107K | 10.78 | 10.23 | 24.46 | 15.16 |
| T8.12-E107Y | 29.68 | 32.34 | 29.38 | 30.47 |
| T8.12-V104M- | 31.16 | 35.99 | 25.03 | 30.73 |
| E107Y | | | | |

To validate the efficacy of T8.12 variants in mammalian cells, the variants were transcribed in vitro into mRNA and co-transfected into Huh7 cells with synthetic sgRNA at an equivalent mass. Huh7 cells (8×10³ cells per well) were plated in a 96-well plate 20-24 hours before transfection. Transfection was carried out with varying doses of mRNA and sgRNA3 as described in Table 29 using lipofectamine MessengerMax Reagent (LMRNA015), following the manufacturer's protocols. After 72 hours, cells were harvested, genomic DNA was extracted, and the editing efficiency was analyzed by NGS. T8.12 variants demonstrated higher on-target editing efficiency compared to unmodified T8.12 (Table 29).

TABLE 29
C-to-T Editing Efficiency of sgRNA3 with T8.12 Variants

| mRNA dose(ng) | | | |
|---|---|---|---|
| mRNA:sgRNA = 1:1 | T8.12 | T8.12-E107Y | T8.12-V104M-E107Y |
| 20 | 87.32 | 87.44 | 88.01 |
| 6.67 | 71.46 | 77.87 | 85.00 |
| 2.22 | 41.35 | 57.72 | 66.89 |
| 0.74 | 17.94 | 27.18 | 36.40 |
| 0.25 | 7.73 | 10.50 | 16.26 |
| 0.08 | 2.98 | 3.30 | 5.73 |

### Example 16: Directed Evolution of T88.74 to Improve its C to T Editing Activity

To improve the activity of T88.74 (SEQ ID NO: 241), a site-saturation mutagenesis library of T88.74 was generated as described above, and this library was subjected to directed evolution. The experimental system utilized three plasmids: a selection plasmid that expressed mutated Chloramphenicol resistant gene (Cam^{R}-L158P, H193R), featuring two inactivation mutations, an sgRNA expression plasmid that expressed two sgRNAs targeting the mutated sites; and an editor plasmid that expressed the T88.74-dCas9-2×UGI library. Library members capable of introducing C·G to T·A edits at all two sites simultaneously would restore Chloramphenicol resistance, thereby enabling survival upon challenge with Chloramphenicol.

In conducting the experiment, E. coli strain DH10B, co-transformed with the selection plasmid and sgRNA expression plasmid, was made electrocompetent. Subsequently, 100-200 ng of the editor plasmid library was electroporated into DH10B cells. The bacteria were then recovered in 10 ml of LB medium before inoculation into 20 ml of LB medium supplemented with three antibiotics for plasmid maintenance (kanamycin 30 μg/mL, ampicillin 100 μg/mL, spectinomycin 150 μg/mL). Additionally, 1% Arabinose was introduced to induce the expression of T88.74-dCas9-2×UGI library.

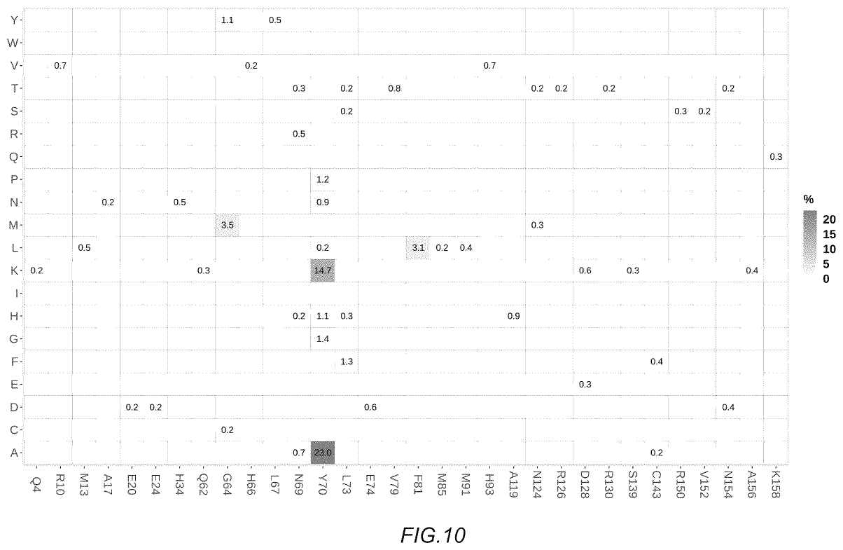

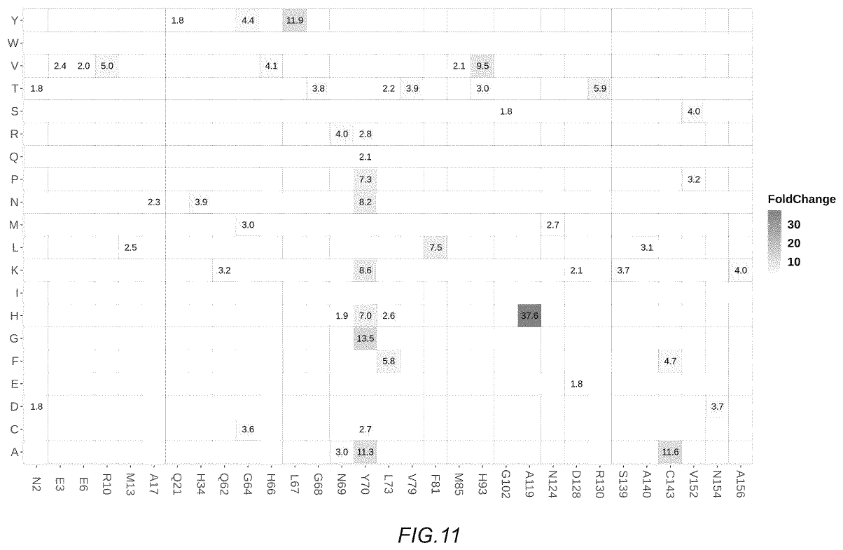

The bacteria were cultured for an additional 16 hours before being plated onto bioassay dishes containing 1.5% agar-2×YT, the plasmid maintenance antibiotic, and various concentrations of Chloramphenicol (16-64 μg/mL). Parallel experiments were conducted on two separate plates. Following incubation at 37° C. overnight, 48 surviving colonies from one plate were individually amplified by PCR and subsequently subjected to Sanger sequencing. At the same time, surviving colonies from the other plate were pooled together and subjected to plasmid extraction. The T88.74 gene library from those surviving colonies were then amplified from these extracted plasmids and subsequently subjected to Next-generation sequencing for comprehensive analysis. The percentage of mutations and the fold change of enrichment was calculated in a similar way in Example 12. The top 50 values are shown in FIG. 10 and FIG. 11.

### Example 17: Directed Evolution of T8.4 to Further Improve its a to G Editing Activity

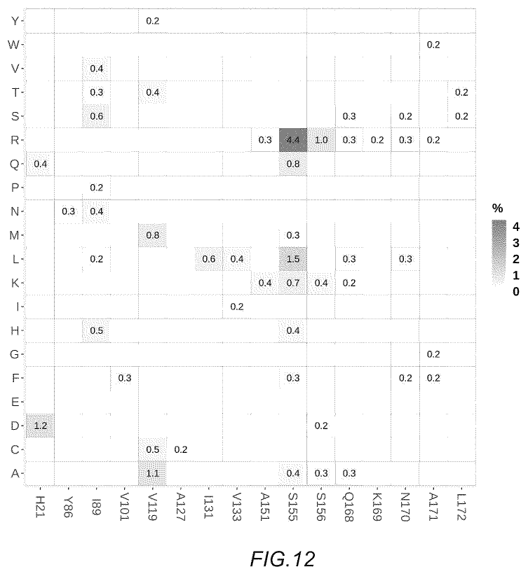

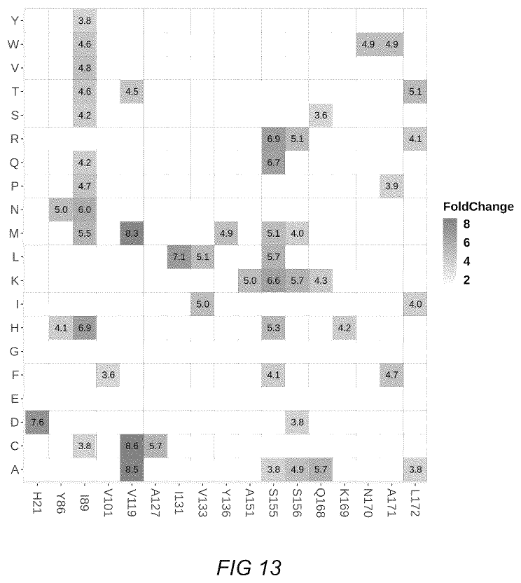

To further improve the activity of T8.4, the original T8.4 NNK library was expanded to contain another 60 NNK mutations across the sequence and subjected to directed evolution. Similar experimental systems were employed to enrich beneficial mutations (detailed KanR mutations). The NGS analysis results of enriched mutants were shown in FIG. 12 and FIG. 13.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An adenosine deaminase comprising an amino acid sequence having at least 90% sequence identity to SEQ ID NO:74.

2. The adenosine deaminase of claim 1, wherein the amino acid sequence has at least 95% sequence identity to SEQ ID NO:74.

3. The adenosine deaminase of claim 1, wherein the amino acid sequence has at least 99% sequence identity to SEQ ID NO:74.

4. The adenosine deaminase of claim 1, wherein the amino acid sequence has the sequence shown in SEQ ID NO:74.

5. The adenosine deaminase of claim 1, wherein the adenosine deaminase comprises one or more alterations at positions S15X, L19X, N30X, A61X, G63X, M81N, V95X, V119X, S122X, S153X, S155X, C159X, E164X, E167X as numbered relative to SEQ ID NO: 74, wherein X is any amino acid.

6. The adenosine deaminase of claim 5, wherein the adenosine deaminase comprises one or more alterations at positions S15G, L19A, N30R, A61C, G63Q, M81N, V95T, V119M, S122R, S153R, S155R, C159R, E164V, E164L, E164W, E167W as numbered relative to SEQ ID NO: 74.

7. The adenosine deaminase of claim 1, wherein the adenosine deaminase is capable of deaminating adenosine in DNA.

8. The adenosine deaminase of claim 1, wherein the adenosine deaminase is capable of deaminating cytidine in DNA.

9. The adenosine deaminase of claim 1, wherein the adenosine deaminase comprises one or more amino acid substitutions selected from the group consisting of W36R, P61A, R64L, L97F, A119V, D121N, A122S, T124R, D132N, H135N, H136Y, S159C, F162Y, R165P, K170N, and D183N as numbered relative to SEQ ID NO: 4.

10. The adenosine deaminase of claim 9, wherein the adenosine deaminase is an adenosine deaminase enzyme having an amino acid sequence of SEQ ID NO: 74.

11. A nucleobase editor comprising (i) a polynucleotide programmable DNA binding domain; and (ii) an adenosine deaminase, wherein the adenosine deaminase has an amino acid sequence having at least 90% sequence identity to SEQ ID NO:74.

12. The nucleobase editor of claim 11, wherein the adenosine deaminase has an amino acid sequence having at least 95% sequence identity to SEQ ID NO:74.

13. The nucleobase editor of claim 11, wherein the adenosine deaminase has an amino acid sequence having at least 99% sequence identity to SEQ ID NO:74.

14. The nucleobase editor of claim 11, wherein the adenosine deaminase has an amino acid sequence having the sequence shown in SEQ ID NO:74.

15. A polynucleotide encoding the adenosine deaminase of claim 1.

16. An expression vector comprising the polynucleotide of claim 15.

17. A method for base editing comprising contacting a polynucleotide sequence with the nucleobase editor of claim 11, wherein the adenosine deaminase deaminates a nucleobase in the polynucleotide, thereby editing the polynucleotide sequence.

18. A method of correcting a genetic defect in a subject, the method comprising administering to the subject: the nucleobase editor of claim 11 or a polynucleotide encoding the nucleobase editor of claim 11 and one or more guide polynucleotides that direct the nucleobase editor to deaminate a target nucleobase in a target nucleotide sequence of the subject, thereby correcting the genetic defect.

19. An adenosine deaminase comprising an amino acid sequence having at least 85% sequence identity to SEQ ID NO:74 and having an Asn at position 121, wherein the adenosine deaminase exhibits DNA deaminase activity.

20. The adenosine deaminase of claim 19, wherein the amino acid sequence has at least 90% sequence identity to SEQ ID NO:74.

21. The adenosine deaminase of claim 19, wherein the amino acid sequence has at least 95% sequence identity to SEQ ID NO:74.

22. The adenosine deaminase of claim 19, wherein the amino acid sequence has at least 99% sequence identity to SEQ ID NO:74.

23. The adenosine deaminase of claim 19, wherein the amino acid sequence has the sequence shown in SEQ ID NO:74.
